(19) United States
(12) Patent Application Publication
(10) Pub. No.: US 2025/0304545 A1
(43) Pub. Date: Oct. 2, 2025

(54) WATER-SOLUBLE HETEROCYCLYL POLYMETHINE CHROMOPHORES

(71) Applicant: The Regents of the University of California, Oakland, CA (US)

(72) Inventors: Ellen M. Sletten, Los Angeles, CA (US); Shang Jia, Los Angeles, CA (US); Cesar Arturo Garcia, Los Angeles, CA (US); Monica Hui Pengshung, Los Angeles, CA (US); Heidi Lee van de Wouw, Los Angeles, CA (US); Emily Mobley, Los Angeles, CA (US)

(21) Appl. No.: 18/836,191

(22) PCT Filed: Feb. 7, 2023
(86) PCT No.: PCT/US2023/062095

§ 371 Date: Aug. 6, 2024

Related U.S. Application Data
(60) Provisional application No. 63/307,509, filed on Feb. 7, 2022.
(60) Provisional application No. 63/402,196, filed on Aug. 30, 2022.

(51) Int. Cl. C07D 311/60; A61K 31/095; A61K 31/352; A61K 31/4745; C07D 335/06; C07D 491/16

(52) U.S. Cl. CPC C07D 311/60; A61K 31/095; A61K 31/352; A61K 31/4745; C07D 335/06; C07D 491/16

(57) ABSTRACT

The present disclosure provides water-soluble and low-aggregation NIR- and SWIR-active small molecule polymethine dyes with improved properties for use in optical imaging, photothermal therapy, and photodynamic therapy.

### RELATED APPLICATIONS

This application claims the benefit of priority to U.S. Provisional Patent Application No. 63/307,509, filed Feb. 7, 2022, and U.S. Provisional Patent Application No. 63/402,196, filed Aug. 30, 2022, the contents of each of which are hereby incorporated by reference in their entirety.

### STATEMENT OF GOVERNMENT SUPPORT

This invention was made with government support under Grant Numbers EB02717 and GM135380, awarded by the National Institutes of Health and under Grant Numbers 1905242 and 2034835, awarded by the National Science Foundation. The government has certain rights in the invention.

### BACKGROUND

Photomedicine broadly refers to the use of light for diagnostic or therapeutic procedures, including optical imaging, photothermal therapy (thermal ablation of cells) and photodynamic therapy (reactive oxygen species induced apoptosis or necrosis). (See Hamblin, M. R.; Huang, Y. Y. Handbook of Photomedicine; CRC Press: Boca Raton, 2014.) The low toxicity of light coupled with the direct control of localization and dosage make phototherapy a promising avenue for increasing the therapeutic index of disease treatment. (See Yuan, A.; Wu, J.; Tang, X.; Zhao, L.; Xu, F.; Hu, Y. J. Pharm. Sci. 2013, 102, 6-28.) Additionally, the inexpensive nature of lasers and detectors poise photoimaging platforms as cost-effective preventative healthcare screening procedures. (See Massoud, T. F.; Gambhir, S. S. Genes Dev. 2003, 17, 545-580.) Despite its potential, photomedicine has encountered a key limitation: the penetration of light into tissue, defined as the point in which ⅔ of the light has been scattered or absorbed by endogenous biomolecules. (Tong, R.; Kohane, D. S. WIREs Nanomed. Nanobiotechnol. 2012, 4, 638-662.) As one moves toward lower energy light, the distance light can traverse through tissue increases. (See Weissleder, R. Nat. Biotechnol. 2001, 19, 316.) This is a well-known phenomenon that has resulted in a large push toward near-infrared (NIR, 700-1000 nm, FIG. 1) chromophores, fluorophores, and activatable probes. (Yuan, A.; Wu, J.; Tang, X.; Zhao, L.; Xu, F.; Hu, Y. J. Pharm. Sci. 2013, 102, 6.) However, short-wave infrared (SWIR, 1000-2000 nm, also referred to as the NIR-II region) probes have not received as much attention, despite the fact that tissue penetration is superior in this region, especially when there is high blood content (FIG. 1). (Lim, Y. T.; Kim, S.; Nakayama, A.; Stott, N. E.; Bawendi, M. G.; Frangioni, J. V. Mol. Imaging 2003, 2, 50.)

IIongjie Dai and coworkers demonstrated using a carbon nanotube (CNT)-NIR-cyanine dye conjugate that the depth and resolution of in vivo imaging is superior above 1000 nm. (Hong, G.; Lee, J. C.; Robinson, J. T.; Raaz, U.; Xie, L.; Huang, N. F.; Cooke, J. P.; Dai, H. Nat. Med. 2012, 18, 1841.) The main limitation was the need for CNTs as a SWIR contrast agent, as there are concerns regarding the biocompatibility of CNTs. (Foldvari, M.; Bagonluri, M. Nanomedicine: Nanotechnol. Biol. Med. 2008, 4, 183.) While the potential of the SWIR has been demonstrated, materials that emit in this region are limited. Most reports of imaging in the SWIR have employed carbon nanotubes or quantum dots. Rare earth nanomaterials, as well as layer-by-layer assembled nanoparticles containing an organic dye have also been employed. However, all these materials are sizeable and do not represent a direct comparison to the fluorophores that have been enormously successful in vitro. What is necessary is the development of bright, stable, non-toxic small-molecule fluorophores that span the SWIR region and will allow for multiplexed imaging experiments. (See Antaris, A. L.; Chen, H.; Cheng, K.; Sun, Y.; Hong, G.; Qu, C.; Diao, S.; Deng, Z.; Hu, X.; Zhang, B.; Zhang, X.; Yaghi, O. K.; Alamparambil, Z. R.; Hong, X.; Cheng, Z.; Dai, H. Nat. Mater. 2015, 15, 235.)

### SUMMARY OF THE INVENTION

While the toolbox of SWIR-emissive fluorophores has grown considerably in the past decade, known SWIR-excitable fluorophores either have low or negligible quantum yields, are prone to aggregation, and/or are too hydrophobic to be used directly for in vivo imaging.

The present disclosure provides water-soluble NIR and SWIR-active small molecules with improved properties for use in optical imaging, photothermal therapy, and photodynamic therapy. It also discloses methods to prevent aggregation in nanomaterial formulations.

Accordingly, the present disclosure provides compounds of formula I:

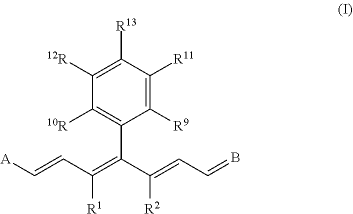

wherein:

R¹ and R² are each independently selected from H, alkyl, or halo; or R¹ and R² together complete a cycloalkenyl ring, a heterocyclyl ring, or a polycyclyl ring system;
R⁹ is II, alkyl (preferably lower alkyl, most preferably methyl), alkoxy (such as methoxy), haloalkyl (such as trifluoromethyl) or halo;
R¹⁰ is H, alkyl (preferably lower alkyl, most preferably methyl), alkoxy (such as methoxy), haloalkyl (such as trifluoromethyl) or halo;
R¹¹ and R¹² are each independently H, C₃-C₁₀ alkyl (such as t-butyl, trifluoromethyl, methyl, or ethyl) or cycloalkyl (such as cyclopropyl); and
R¹³ is H, alkynyl-alkyl (such as propargyl), alkynyl, heteroaralkyl, heteroaryl, a group comprising an azide (such as azido acetate or azidoalkyl), heteroaralkyl, or a moiety that comprises a reactive group, e.g., capable of undergoing bioconjugation, such as an N-hydroxysuccinimide ester or pentafluorophenyl ester, or a group comprising an acid, aldehyde, aide, alkyne, alkene, hydroxyl, amide, urea or sulfonamide; and
   A and B are each independently selected from a bicyclic, tricyclic, or tetracyclic heteroaryl;
wherein A and B are each independently substituted with one or more R^{14a} and/or R^{14b};
   wherein each R^{14a} and/or R^{14b} is independently selected from H, alkoxy, acyl, heteroaryl, sulfonate, carbonate, cyano, ester, amide, halo, aryl, amino, alkylamino, C₁₋₆ alkyl, C₃₋₁₀ cycloalkyl, haloalkyl, aralkenyl (preferably arylethenyl), aralkynyl (preferably arylethynyl), hetaralkenyl (preferably heteroarylethenyl), hetaralkynyl (preferably heteroarylethynyl), and heterocyclyl;
   or two adjacent R^{14a} and/or R^{14b} groups combine to form a carbocyclic or heterocyclic ring including the atoms to which they are attached.

The present disclosure also provides methods of using these dyes for in vivo sensing or cargo delivery.

### BRIEF DESCRIPTION OF THE DRAWINGS

FIG. 1 is a scheme showing exemplary syntheses and retrosyntheses of flavylium and chromenylium polymethine fluorophores. (A) Heptamethine dyes with a cyclohexyl linker. (B) Flavylium and chromenylium heterocycles. (C/D) Suzuki chemistry on heptamethine dyes (C) or linker (D) with R₃═Cl. See Tables at end of document for scope of R₁, R₂, R₃.

FIG. 2 depicts some of the dyes of the disclosure and their respective syntheses (A); absorption and emission spectra in fetal bovine serum (FBS) (B); and comparative brightness imaging on an InGaAs camera with 1 μM solutions of ICG, AmmonChrom7, SulfoChrom7, and ZwitChrom7 excited at 795, 890, and 980 nm (C). A representative image with 980 nm excitation is shown (top) and the plot represents quantification of all images calibrated to counts/exposure time. (D) In vivo imaging of AmmonChrom7 (100 nmol) injected i.v. 980 nm Ex, 0.5 ms, LP=1100 nm.

FIG. 3 depicts (A) Proposed water soluble conjugatable fluorophores and (B) their application to molecular imaging and biomolecule tracking. Red triangle=targeting agent. (C) Recently prepared SChrom7 and (D) comparative absorption and emission to JuloFlav7 demonstrating the potential for SChrom7 to be an excellent contrast agent for 1064 nm excitation.

FIG. 4 depicts chromenylium star (CStar) polymer contrast agents. (A) Synthesis of star polymer through click chemistry of alkyne-functionalized chromenylium dye and azide-terminated poly(2-methyl-2-oxazoline)s. (B) The absorbance and emission spectra of 3 synthesized CStar polymers with different length arms after dialysis. A small amount of trimethine is also present. (C) In vivo images taken 2 min after injection of CStar30 showing little accumulation in the liver (left) and strong kidney signal (right). 980 nm Ex, 20 ms, LP=1100 nm.

FIG. 5 is a drawing depicting procedure for incorporation of small molecule dyes into micellar structures, preparing micelle formulations.

FIG. 6 demonstrates that adding steric bulk to phenyl derivatives further decreases H-aggregation. DitBuPh Flav7 is mostly monomeric (free) dye and a good candidate for SWIR imaging.

FIG. 7 depicts brightness of several dyes of the disclosure measured by SWIR imaging using 974 nm excitation light with different filters. Flav7 derivatives have similar brightness despite decreasing quantum yield (QY).

FIG. 8 demonstrates that increasing steric bulk is a viable strategy to reduce H-aggregation in micelle formulations.

FIG. 9 displays the results of SWIR experiments which indicate the DitBuPh-Julo7 is mostly monomeric in micelles.

FIG. 10 demonstrates that DitBuPh-Julo7 is slightly brighter than Julo7 in micelles at excitation wavelength=1060 nm.

FIG. 11 displays the results of photophysical aggregation studies of exemplary compounds of the disclosure. Dashed lines indicate photophysical data for aggregated dyes in solution, and solid lines indicate data for non-aggregated dyes in solution.

FIG. 12 depicts the results of imaging studies conducted in a mouse using Sulfo-Chrom7 as a contrast agent.

FIG. 13 depicts the organ-specific results of imaging studies conducted in a mouse using Sulfo-Chrom7 as a contrast agent.

FIG. 14 depicts the design of hydrophilic and versatile Chrom7 derivatives. (a) Water soluble polymethine dyes used for SWIR imaging and their emission wavelengths. (b) Structure of ICG. (c) Structures of Flav7 and Chrom7. (d) Derivatization of PropChrom7 into a series of water soluble, functional SWIR imaging agents.

FIG. 15 depicts the synthesis of PropChrom7 and its post-synthetic CuAAC to afford SulfoChrom7, AmmonChrom7, ZwitChrom7.

FIG. 16 depicts photophysical comparisons of water soluble SWIR fluorophores. (a) Table of photophysical properties. See FIG. S5a for error values. (b) Brightness comparison by SWIR imaging at 4 ms/frame of each water soluble dye (2 μM) in FBS in capillaries under 975 nm illumination (100 mW/cm²), with 2 μM of ICG in FBS under 785 nm illumination (50 mW/cm²) as a benchmark. (c) Quantification of (b). (d,e) Overlay of absorption (solid line) and emission (dotted line) spectra of 2 μM Sulfo-, Ammon- or ZwitChrom7 in methanol (d) or FBS (e).

FIG. 17 depicts video-rate imaging of mouse vasculature with of i.v. injected SulfoChrom7. (a) Schematics of i.v. injection of SulfoChrom7 for vasculature imaging under different filters. (b-d) Fluorescence images of mice recorded at 30 ms/frame (b) under 1100 nm LP filter with 0.05 nmol dye (20 frames averaged, max brightness 646 excluding tail region), or (c) under 1400 nm LP filter with 5.0 nmol dye (20 frames averaged, max brightness 289 excluding tail region); their raw brightness profiles along the highlighted line is shown in (d). (e) Schematics of co-injection of SulfoChrom7 and ICG for their direct comparison under two-channel imaging. (f-h) Fluorescent images of a mouse at 10 ms/frame under 1300 nm LP filter (f) 3 min [84 frames averaged, max brightness 4302 (green) and 3960 (blue)] or (g) 32 min [87 frames averaged, max brightness 3692 (green) and 4027 (blue)] after i.v. injection of SulfoChrom7 (10 nmol, shown in green) and ICG (10 nmol, shown in blue); their raw brightness profiles along the highlighted line is shown in (h). Illumination was provided at 100 mW/cm² for 975 nm and 50 mW/cm² for 785 mm.

FIG. 18 depicts the tracking of tumor xenograft growth using AmmonChrom7. (a) Schematics of s.c. injection of AmmonChrom7 stained cells for tracking of their in vivo growth. (b) Uptake and retention of AmmonChrom7 in different cell lines. (c) Comparison of the growth curves of stained A375 as determined by fluorescence imaging and by caliper measurement in three mice. (d) In vivo images of A375 tumors 34 days after xenograft on Mouse 1 (1 ms/frame, 31 frames averaged, max brightness 7957). (e, f) Ex vivo fluorescent images of A375 tumors and organs (e) and carcass (f) of Mouse 1 34 days after xenograft [0.8 ms/frame, 51 frames averaged, max brightness 10873 (e) and 2920 (f)]. See FIG. S12e,f for corresponding bright field images. Lu: lung, Sp: spleen, St: stomach, In: intestine, He: heart, Ste: sternum, Li: liver, Ki: kidney. TuL: left, stained tumor. TuR: right, unstained tumor. Sc: subcutaneous tissue around TuL. (g) Maximum brightness of ex vivo fluorescent tumor, liver and subcutaneous tissue surrounding the tumor (mean±s.d., Mouse 1:34 days, Mouse 2:15 days, Mouse 3:23 days after xenograft). (h) Ex vivo fluorescent images of SK-OV-3 tumors and organs 39 days after xenograft and 4 h after i.v. injection of OTL-38 [1 ms/frame, 112 frames averaged, max brightness 9928 (green) and 2095 (blue)]. See FIG. S13g for corresponding bright field image. Tu1: abdomen s.c. tumor. Tu2: flank s.c. tumor. (i) Maximum fluorescence intensity of ex vivo tumors and their surrounding hypodermis after tumor removal from the sacrificed mice (n=3, mean±s.d., 39 days after xenograft). Illumination was provided at 100 mW/cm² for 975 nm and 50 mW/cm² for 785 mm. Scale bar: 2 cm.

FIG. 19 depicts imaging of bone using PhosphoChrom7. (a) Schematics of bone imaging by s.c. injection of PhosphoChrom7. (b) Absorption and emission spectra of 2 μM PhosphoChrom7 in FBS. (c) Fluorescent image of calcium hydroxyapatite suspension in bovine serum treated with PhosphoChrom7, and its subsequent washes with bovine serum (0.5 ms/frame, 101 frames averaged, max brightness 11338); quantification is shown below (mean±s.d., n=3). (d-f) Fluorescence images of a mouse 24 h after i.v. injection of 20 nmol PhosphoChrom7 on (d) ventral, (e) dorsal or (f) lateral view. M: mandible, Mx: maxilla, R: Rib, Ste: sternum, T: tibia, P: phalange, V: vertebrae, C: carpus, E: elbow, Kn: knee. (g) Representative single frame from Video S3 showing fluorescence image of awake mice 48 h after injection. (h) Fluorescence images of a mouse after removal of abdominal organs and most of skin on dorsal view. Images were captured under 100 mW/cm² 975 nm illumination at 3 ms/frame (d-h); 81 frames (d-e), single frame (f-g) or 201 frames (h) averaged. Max brightness: 7096 (d), 4985 (e), 6379 (f), 6962 (g) and 12313 (h).

FIG. 20 depicts the absorption and emission spectra of PropChrom7 (2 μM) in MeOH, normalized to the maximum intensity.

FIG. 21 depicts the photophysical characterization of SulfoChrom7. (a-c) Absorption and emission spectra of SulfoChrom7 (2 μM) in (a) MeOH, (b) water, and (c) fetal bovine serum (FBS). (d) Absorption spectra of different concentrations of SulfoChrom7 in H₂O. (e) Absorption spectra of increasing concentrations of SulfoChrom7 using 2 mm light path. Absorption spectra in (d) and (e) are normalized to the monomer peak.

FIG. 22 depicts the photophysical characterization of AmmonChrom7. (a-c) Absorption and emission spectra of AmmonChrom7 (2 μM) in (a) MeOH, (b) water, and (c) fetal bovine serum. (d) Absorption spectra of different concentrations of AmmonChrom7 in H₂O. (e) Absorption spectra of increasing concentrations of AmmonChrom7 using 2 mm light path, normalized to the monomer absorption peak. Absorption spectra in (d) and (e) are normalized to the monomer peak.

FIG. 23 depicts the photophysical characterization of ZwitChrom7. (a-c) Absorption and emission spectra of ZwitChrom7 (2 μM) in (a) MeOH, (b) water, and (c) fetal bovine scrum. (d) Absorption spectra of different concentrations of ZwitChrom7 in H₂O. (e) Absorption spectra of increasing concentrations of ZwitChrom7 using 2 mm light path. Absorption spectra in (d) and (e) are normalized to the monomer peak.

FIG. 24 depicts the in vitro comparison among Sulfo-, Ammon-, Zwit- and PhosphoChrom7. (a) Table of photophysical properties of all dyes in this work. Flav7 (Φ_{F}=0.61%) was used as a reference for fluorescent quantum yield. (b) Stability of 1 μM dyes in FBS upon 37° C. incubation (mean±s.d., n=3). (c) Cytotoxic effects of dyes on HEK293 cell proliferation over 18 h determined by MTT assay (mean±s.d., n=3). (d) Comparison of absorption spectra of 2 μM Sulfo-, Ammon- and ZwitChrom7 in H₂O, PhosphoChrom7 in 25 mM HEPES buffered at pH 7.5, and previously reported water soluble SWIR polymethine dyes ICG-C11 in H₂O (1% DMSO), FD-1080, LZ-1105 and FNIR-1072 in PBS (re-plotted from the original reports).¹⁻³ Solid arrows point to the monomer peak and dashed arrows point to the putative aggregation peak. Ground state desymmetrization peaks usually appear more blue-shifted, but need verification against aggregation, which is concentration dependent.

FIG. 25 depicts the distribution of SulfoChrom7 in mice over time. (a-d) Fluorescent images of mouse (a) immediately, (b) 3 h, (c) 24 h, (d) 48 h after i.v. injection of 20 nmol of SulfoChrom7. (e) Fluorescent image of organs dissected 48 h after i.v. injection. (f) Distribution of SulfoChrom7 in different organs 48 h after i.v. injection, estimated and normalized by the integrated intensity (mean #s.d., n=3). (g) Change of mean fluorescence intensity in mouse body over time; pixels with brightness higher than 200 were calculated (mean #s.d., n=3). Images shown are acquired from one representative mouse of three replicates. Lu: lung, Sp: spleen, St: stomach, In: intestine, IIe: heart, Ste: sternum, Li: liver, Ki: kidney. See FIG. 37 for acquisition and processing parameters.

FIG. 26 depicts the distribution of AmmonChrom7 in mice over time. (a-d) Fluorescent images of mouse (a) immediately, (b) 3 h, (c) 24 h, (d) 48 h after i.v. injection of 20 nmol of AmmonChrom7. (e) Fluorescent images of organs dissected 48 h after i.v. injection. (f) Distribution of AmmonChrom7 in different organs 48 h after i.v. injection, estimated and normalized by the integrated intensity (mean±s.d., n=3). (g) Change of mean fluorescence intensity in mouse body over time; pixels with brightness higher than 200 were calculated (mean #s.d., n=3). Images shown are acquired from one representative mouse of three replicates. Lu: lung, Sp: spleen, St: stomach, In: intestine, He: heart, Ste: sternum, Li: liver, Ki: kidney. See FIG. 37 for acquisition and processing parameters.

FIG. 27 depicts the distribution of ZwitChrom7 in mice over time. (a-d) Fluorescent images of mouse (a) immediately, (b) 3 h, (c) 24 h, (d) 48 h after i.v. injection of 20 nmol of ZwitChrom7. (e) Fluorescent images of organs dissected 48 h after i.v. injection. (f) Distribution of ZwitChrom7 in different organs 48 h after i.v. injection, estimated and normalized by the integrated intensity (mean±s.d., n=3). (g) Change of mean fluorescence intensity in mouse body over time; pixels with brightness higher than 200 were calculated (mean #s.d., n=3). Images shown are acquired from one representative mouse of three replicates. Lu: lung, Sp: spleen, St: stomach, In: intestine, He: heart, Ste: sternum, Li: liver, Ki: kidney. See FIG. 37 for acquisition and processing parameters.

FIG. 28 depicts fast mouse imaging with low amount of SulfoChrom7. (a-e) Fluorescent images of Mouse 1 under 1100 nm long-pass filter after i.v. injection of increasing amount of SulfoChrom7, and (e) average brightness in body, liver and vein (highlighted regions) over time; arrows point at injection events. (f) Quantification of Mouse 2 under identical conditions. (g-j) Fluorescent images of Mouse 3 under 1400 nm long-pass filter after i.v. injection of increasing amount of SulfoChrom7, and (k) average brightness in body, liver and vein (highlighted regions) over time; arrows point at injection events. (1) Quantification of Mouse 4 under identical conditions. All images were acquired at 33 fps and are shown as an average of 20 sequential frames. See FIG. 37 for acquisition and processing parameters.

FIG. 29 depicts dual-channel imaging of i.v. injected SulfoChrom7 and ICG (10 nmol of each dye) over time and under varying long-pass filters. (a-b, e-f, i-j) Images of a mouse (a) 0 min, (b) 30 min, (e) 3 min, (f) 32 min, (i) 6 min, (j) 33 min after injection under (a-b) 1100 nm, (c-f) 1300 nm, or (i-j) 1400 nm long-pass filter. (e, g, k) Mean brightness ratio between the SulfoChrom7 channel and ICG channel in the area of interest highlighted in (j). (d, h, 1) Replicates of (c), (g) and (k) in another mouse. Bar graphs are shown as mean⊥s.d. across the >200 frames captured at each time point. See FIG. 37 for acquisition and processing parameters.

FIG. 30 depicts a comparison of cellular uptake of dyes. (a) Uptake of Ammon-, Sulfo- and ZwitChrom7 in HEK293 cells (mean±s.d., three biological replicates). (b) Representative absorption spectra in cell lysates.

FIG. 31 depicts FIG. S12 Tracking growth of A375 tumor xenograft using AmmonChrom7. (a-d) In vivo images of A375 tumors in (a) Mouse 1 after 0 day, (b) Mouse 1 after 15 days, (c) Mouse 2 after 15 days, and (d) Mouse 3 after 23 days; non-stained tumor is on the right flank and stained tumor is on the left flank. The arrow in (a) points to the needle mark. (e,f) Ex vivo fluorescent and bright field images of (e) A375 tumors and organs and (f) carcass of Mouse 1 34 days after xenograft; the same acquisition and brightness adjustment parameters were used. Lu: lung, Sp: spleen, St: stomach, In: intestine, He: heart, Ste: sternum, Li: liver, Ki: kidney. TuL: left, stained tumor, TuR: right, non-stained tumor. (g) Change of mean fluorescence intensity over time of the stained A375 tumors on the three mice. Brightness of pixels above ⅕ of maximum brightness is normalized to 1 ms exposure time for averaging. (h) Growth curves of the stained and non-stained A375 tumors on the three mice as monitored by caliper measurement. Scale-bar: 2 cm. See FIG. 37 for acquisition and processing parameters.

FIG. 32 depicts the FIG. S13 Tracking of growth of SK-OV-3 tumor xenograft using AmmonChrom7. (a) Dorsal and (b) ventral images of a mouse 39 days after xenograft and 4 h after i.v. injection of OTL-38. Images are from one representative mouse of three replicates. (c) Brightness comparison by SWIR imaging of bovine serum without, with 2 μM ICG or with 2 μM OTL-38 under 975 nm or 785 nm illumination. (d-f) Change of size and mean fluorescence intensity of two SK-OV-3 tumors over time on three mice. Brightness of pixels above ⅓ of maximum brightness are normalized to 1 ms exposure time for averaging. (g-i) Ex vivo fluorescent and bright field images of (e) A375 tumors and organs, (f) dorsal side and (g) ventral side of carcass of the mouse from FIG. S13a,b 39 days after xenograft and 4 h after i.v. injection of OTL-38; the same acquisition and brightness adjustment parameters were used. Lu: lung, Sp: spleen, St: stomach, In: intestine, He: heart, Ste: sternum, Li: liver, Ki: kidney. Tu1: flank tumor, Tu2: abdomen tumor. Scale-bar: 2 cm. See FIG. 37 for acquisition and processing parameters.

FIG. 33 depicts the water solubility of PhosphoChrom7. (a) Absorption spectra of different concentrations of PhosphoChrom7 in FBS. (b) Absorption spectra of different concentrations of PhosphoChrom7 in 25 mM HEPES buffer, pII 7.5.

FIG. 34 depicts the analysis of PhosphoChrom7 binding on hydroxyapatite by scanning electron microscope (SEM). (a-e) Images of PhosphoChrom7 treated hydroxyapatite: (a) SEM image, and energy dispersive X-ray spectrometry (EDS) mapping of (b) carbon, (c) calcium, (d) oxygen and (e) phosphorus. (f-j) Images of non-treated hydroxyapatite: (f) SEM image, and EDS mapping of (g) carbon, (h) calcium, (i) oxygen and (j) phosphorus. Images of the same element are contrast-enhanced with the same parameters without saturated pixels. Residual carbon signals from (g) may come from remaining methanol or calcium carbonate impurity. Scale-bar: 5 μm.

FIG. 35 depicts the imaging of bone in mice using PhosphoChrom7. (a-d, f-i) Fluorescent images of PhosphoChrom7 in mice (a, f) immediately, (b, g) 8 h, (c, h) 24 h, (d, i) 48 h after i.v. injection of 20 μmol dye, viewed from (a-d) ventral and (f-i) dorsal side. (e, j) Fluorescence images of the skinned mouse in FIG. 7e-f before removal of abdominal organs on (c) ventral and (j) dorsal view. (k) Fluorescence images of a mouse after removal of abdominal organs and most of skin on ventral view. (1) Fluorescent images of organs dissected 54 h after i.v. injection (Lu: lung, Sp: spleen, St: stomach, In: intestine, He: heart, Ste: sternum, Li: liver, Ki: kidney). (m) Distribution of PhosphoChrom7 in different organs 54 h after i.v. injection, estimated and normalized by the integrated intensity in each dissected organ (n=3, mean±s.d.). (n) Change of mean fluorescence intensity in mouse body over time; pixels with raw brightness higher than 200 were calculated (mean±s.d., n=3). All images are of one representative mouse of three replicates (except e, j where n=1). See FIG. 37 for acquisition and processing parameters.

FIG. 36 depicts the Sensitivity-related parameters for certain fluorescent images disclosed herein.

### DETAILED DESCRIPTION OF THE INVENTION

In certain aspects, the present disclosure provides compounds of formula I:

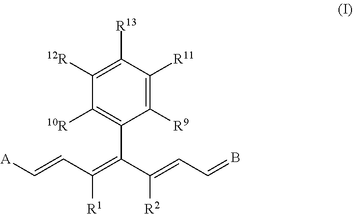

wherein:
R¹ and R² are each independently selected from H, alkyl, or halo; or R¹ and R² together complete a cycloalkenyl ring, a heterocyclyl ring, or a polycyclyl ring system;
R⁹ is H, alkyl (preferably lower alkyl, most preferably methyl), alkoxy (such as methoxy), haloalkyl (such as trifluoromethyl) or halo;
R¹⁰ is H, alkyl (preferably lower alkyl, most preferably methyl), alkoxy (such as methoxy), haloalkyl (such as trifluoromethyl) or halo;
R¹¹ and R¹² are each independently H, C₃-C₁₀ alkyl (such as t-butyl, trifluoromethyl, methyl, or ethyl) or cycloalkyl (such as cyclopropyl); and
R¹³ is H, alkynyl-alkyl (such as propargyl), alkynyl, heteroaralkyl, heteroaryl, a group comprising an azide (such as azido acetate or azidoalkyl), heteroaralkyl, or a moiety that comprises a reactive group, e.g., capable of undergoing bioconjugation, such as an N-hydroxysuccinimide ester or pentafluorophenyl ester, or a group comprising an acid, aldehyde, alkene, hydroxyl, amide, urea or sulfonamide; and
   A and B are each independently selected from a bicyclic, tricyclic, or tetracyclic heteroaryl;
wherein A and B are each independently substituted with one or more R^{14a} and/or R 14b.
   wherein each R^{14a} and/or R^{14b} is independently selected from H, alkoxy, acyl, heteroaryl, sulfonate, carbonate, cyano, ester, amide, halo, aryl, amino, alkylamino, C₁₋₆ alkyl, C₃₋₁₀ cycloalkyl, haloalkyl, aralkenyl (preferably arylethenyl), aralkynyl (preferably arylethynyl), hetaralkenyl (preferably heteroarylethenyl), hetaralkynyl (preferably heteroarylethynyl), and heterocyclyl;
   or two adjacent R^{14a} and/or R^{14b} groups combine to form a carbocyclic or heterocyclic ring including the atoms to which they are attached.

In certain embodiments, the compound has structure of formula Ia:

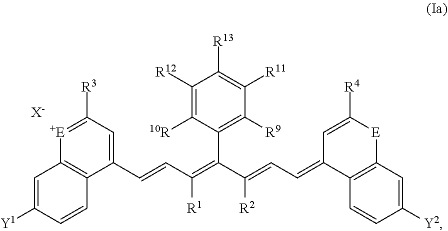

wherein:

### E is O or S;

X is selected from halide and BF₄⁻ perchlorate, B (aryl)₄⁻, boron clusters (e.g., a borohydride complex), and TRISPHAT (tetrabutylammonium phosphorus(V) tris(tetrachlorocatecholate));
Y¹ and Y² are each independently H or Y¹ is —N(R⁵)(R⁶) and Y² is —N(R⁷)(R⁸)
R³ and R⁴ are each independently H, optionally substituted phenyl (preferably phenyl), optionally substituted heteroaryl, alkyl (such as C₃-C₈ alkyl or trifluoromethyl), or cycloalkyl (such as C₃-C₁₀ cycloalkyl, e.g. adamantyl);
each R⁵, R⁶, R⁷ and R⁸, when present, are each independently alkyl, alkynyl-alkyl (such as propargyl), alkynyl, heteroaralkyl, heteroaryl, a group comprising an azide (such as azido acetate or azidoalkyl) or a moiety that comprises a reactive group, e.g., capable of undergoing bioconjugation, such as an N-hydroxysuccinimide ester or pentafluorophenyl ester, or a group comprising an acid, aldehyde, alkene, hydroxyl, amide, urea or sulfonamide.

In certain embodiments, E is O. In other embodiments, E is S.

In certain embodiments, the compounds have the structure of formula Ia:

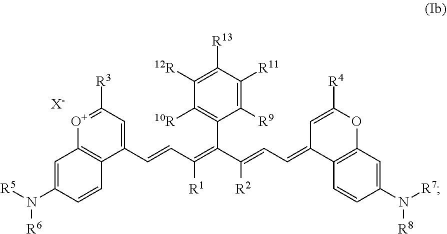

wherein X is selected from halide and BF₄⁻.

In certain embodiments, R¹ and R² together complete a cycloalkenyl ring.

In certain embodiments, R³ and R⁴ are phenyl. In other embodiments, R³ and R⁴ are t-butyl.

In certain embodiments, Y¹ is —N(R⁵)(R⁶) and Y² is —N(R⁷)(R⁸). In other embodiments, Y¹ and Y² are both H.

In certain embodiments, R⁵, R⁶, R⁷ and R⁸ are methyl.

In certain embodiments, the compound has a structure given by formula Ic:

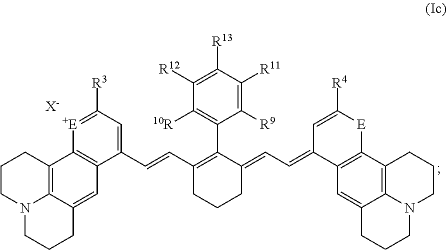

wherein X⁻ is selected from halide and BF₄⁻.

In certain embodiments, R¹¹ and R¹² are H, optionally substituted linear or branched alkyl (such as C₃-C₈ alkyl or trifluoromethyl), or cycloalkyl (such as cyclopropyl).

In certain embodiments, R¹¹ and R¹² are H. In other embodiments, R¹¹ and R¹² are optionally substituted linear or branched alkyl. In certain embodiments, R¹¹ and R¹² are methyl. In certain embodiments, R¹¹ and R¹² are ethyl. In certain embodiments, R¹¹ and R¹² are t-butyl. In c embodiments, R¹¹ and R¹² are cycloalkyl. In certain embodiments, R¹¹ and R¹² are cyclopropyl. In certain embodiments, R¹¹ and R¹² are trifluoromethyl. In certain embodiments, R³ and R⁴ are phenyl. In certain embodiments, R³ and R⁴ are tert-butyl.

In certain embodiments, R¹⁰ is H.

In certain embodiments, R¹¹ and R¹² are each H.

In certain embodiments, R¹³ is H.

In certain embodiments, the compounds have a structure of formula Id:

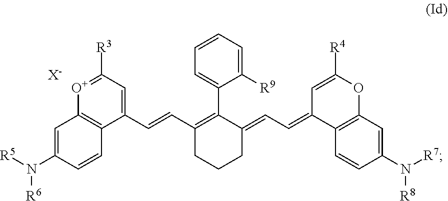

wherein X is selected from halide and BF₄⁻.

In certain embodiments, R³ and R⁴ are each t-butyl.

In certain embodiments, R⁹ is methyl.

In certain embodiments, the compounds have a structure of formula II:

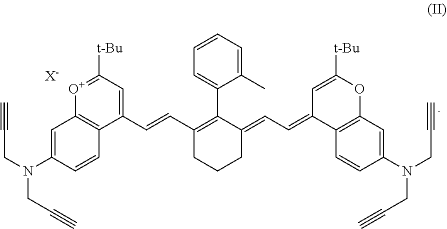

In certain embodiments, X is selected from halide and tetrafluoroborate.

In certain embodiments, at least one of R⁵, R⁶, R⁷ and R⁸ comprises a water-solubilizing group.

In certain embodiments, the compounds have a structure of formula III:

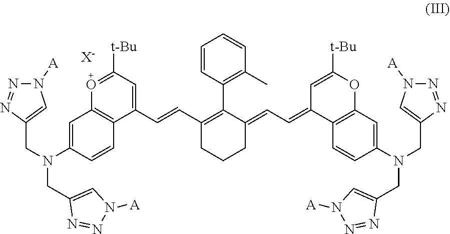

wherein each A comprises a hydrophilic group.

In certain embodiments, the hydrophilic group comprises a carboxylic acid group, an azide-functionalized peptide for targeting, a group that enhances cell permeability, a water solubilizing group or an ionic group. In certain embodiments, the ionic group is sulfate or tetralkylammonium.

In certain embodiments, each A is independently selected from:

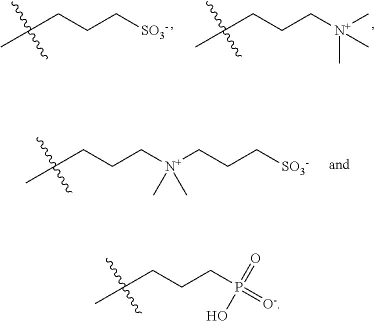

In certain embodiments, the hydrophilic group comprises a hydrophilic oligomer or polymer, such as poly(ethylene glycol) or poly(oxazoline) (e.g., poly(methyl-2-oxazoline).

In certain embodiments, the poly(oxazoline) is selected from P(MeOx)n, P(EtOx)n, P(MeOx)n-block-(PrOx)n, P(EtOx)n-block-(PrOx)n, P(MeOx)n-block-(NonOx) n and P(EtOx)n-block-(NonOx)n.

In certain embodiments, the compound has a structure of formula IV:

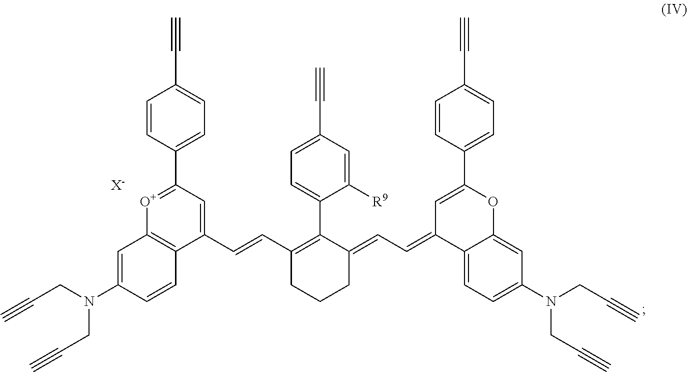

wherein X is selected from halide, BF₄⁻, and perchlorate, B(aryl)₄⁻, boron clusters, and TRISPHAT (tetrabutylammonium phosphorus(V) tris(tetrachlorocatecholatc)); or a click conjugate thereof.

In certain embodiments, the compound has a structure selected from:

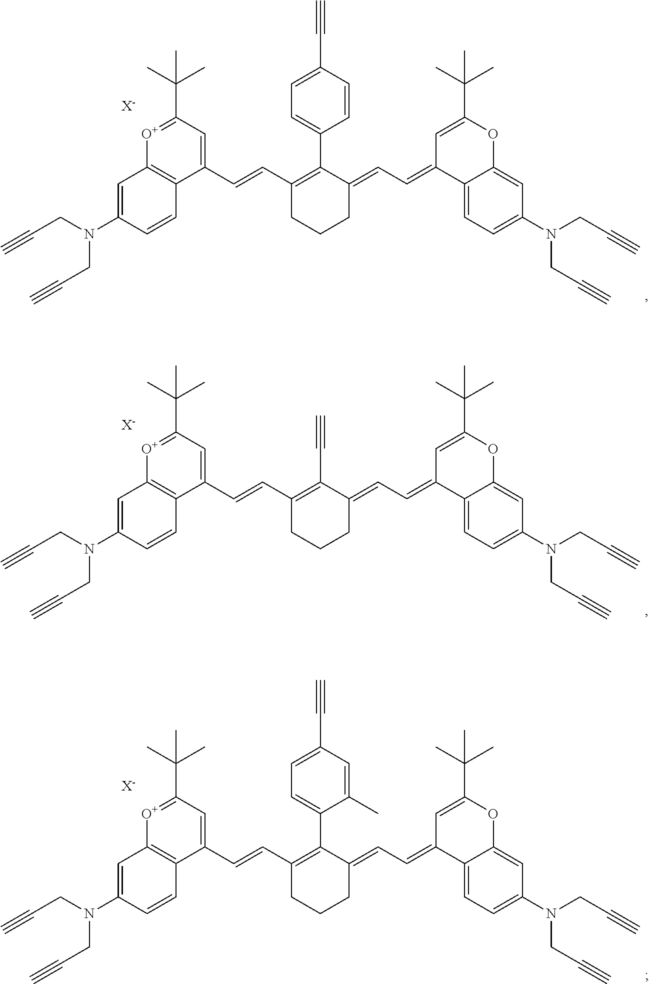

wherein X is selected from halide, BF₄⁻, perchlorate, BAr₄⁻, boron clusters (e.g., a borohydride complex), and TRISPHAT (tetrabutylammonium phosphorus(V) tris(tetrachlorocatecholate));
or a click conjugate thereof.

In certain embodiments, X⁻ is BF₄⁻.

In certain embodiments, the disclosure provides dyes of the following structures:

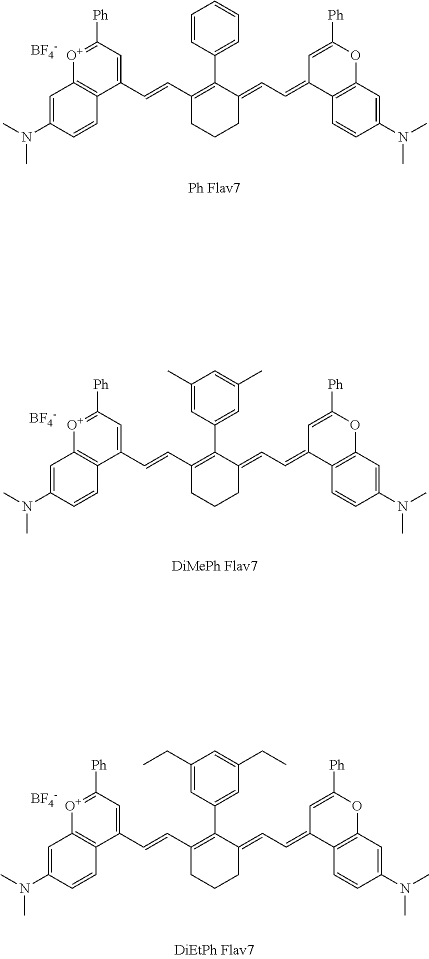

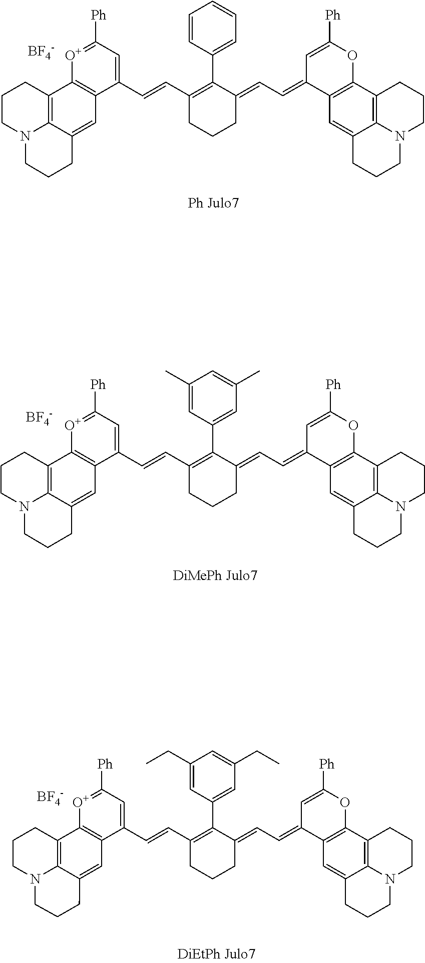 [SMILES: C1=C(c2ccccc2)Oc2c(cc3c4c2CCCN4CCC3)/C1=C/C=C1\CCCC(/C=C/c2cc(-c3ccccc3)[o+]c3c4c5c(cc23)CCCN5CCC4)=C1c1ccccc1 ; "BF4-" ; Cc1cc(C)cc(C2=C(/C=C/c3cc(-c4ccccc4)[o+]c4c5c6c(cc34)CCCN6CCC5)CCC/C2=C\C=C2/C=C(c3ccccc3)Oc3c2cc2c4c3CCCN4CCC2)c1 ; "BF4-" ; CCc1cc(CC)cc(C2=C(/C=C/c3cc(-c4ccccc4)[o+]c4c5c6c(cc34)CCCN6CCC5)CCC/C2=C\C=C2/C=C(c3ccccc3)Oc3c2cc2c4c3CCCN4CCC2)c1 ; "BF4-"]

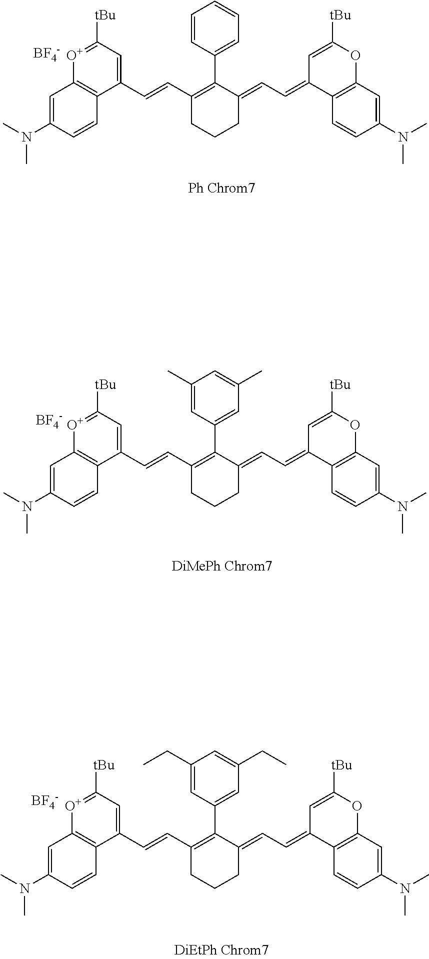

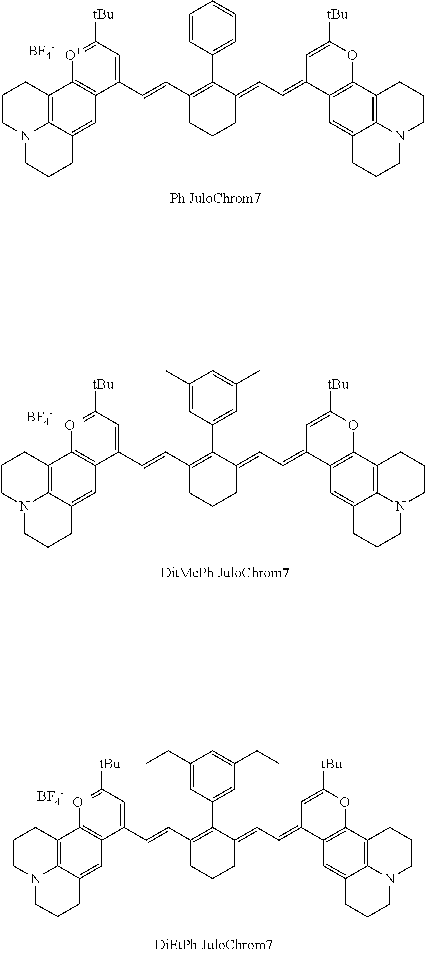 [SMILES: CC(C)(C)C1=C/C(=C\C=C2/CCCC(/C=C/c3cc(C(C)(C)C)[o+]c4c5c6c(cc34)CCCN6CCC5)=C2c2ccccc2)c2cc3c4c(c2O1)CCCN4CCC3 ; "BF4-" ; Cc1cc(C)cc(C2=C(/C=C/c3cc(C(C)(C)C)[o+]c4c5c6c(cc34)CCCN6CCC5)CCC/C2=C\C=C2/C=C(C(C)(C)C)Oc3c2cc2c4c3CCCN4CCC2)c1 ; "BF4-" ; CCc1cc(CC)cc(C2=C(/C=C/c3cc(C(C)(C)C)[o+]c4c5c6c(cc34)CCCN6CCC5)CCC/C2=C\C=C2/C=C(C(C)(C)C)Oc3c2cc2c4c3CCCN4CCC2)c1 ; "BF4-"]

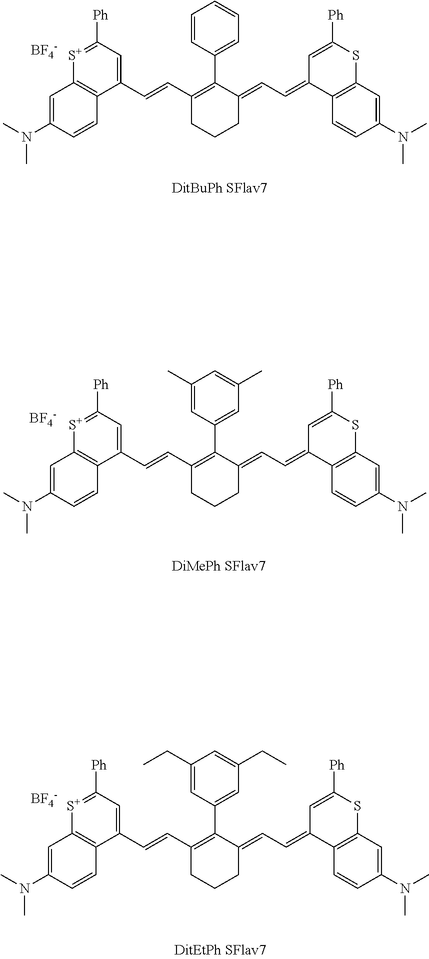

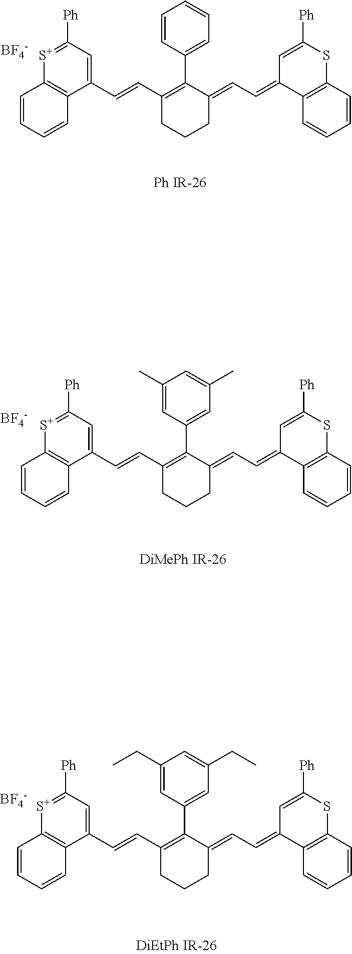 [SMILES: "BF4-" ; C1=C(c2ccccc2)Sc2ccccc2/C1=C/C=C1\CCCC(/C=C/c2cc(-c3ccccc3)[s+]c3ccccc23)=C1c1ccccc1 ; "BF4-" ; Cc1cc(C)cc(C2=C(/C=C/c3cc(-c4ccccc4)[s+]c4ccccc34)CCC/C2=C\C=C2/C=C(c3ccccc3)Sc3ccccc32)c1 ; "BF4-" ; CCc1cc(CC)cc(C2=C(/C=C/c3cc(-c4ccccc4)[s+]c4ccccc34)CCC/C2=C\C=C2/C=C(c3ccccc3)Sc3ccccc32)c1]

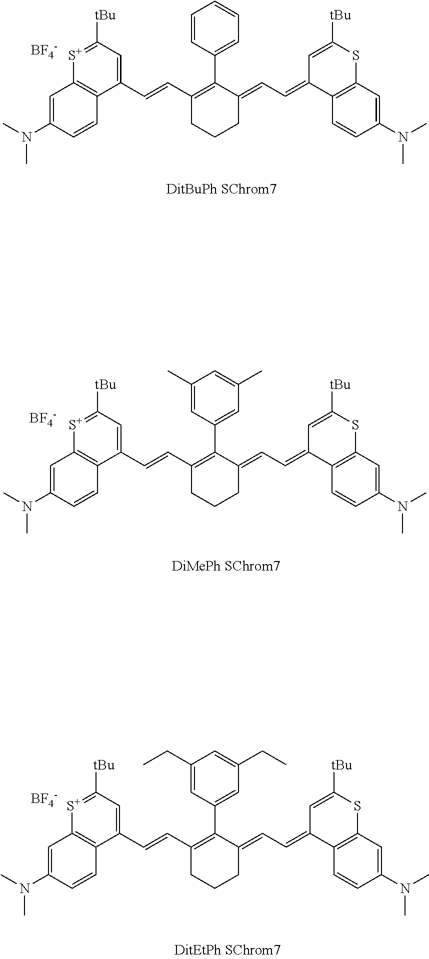

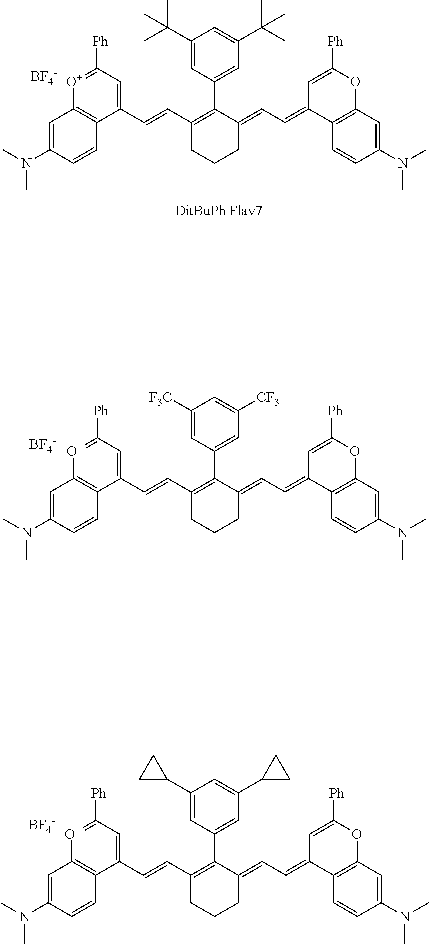

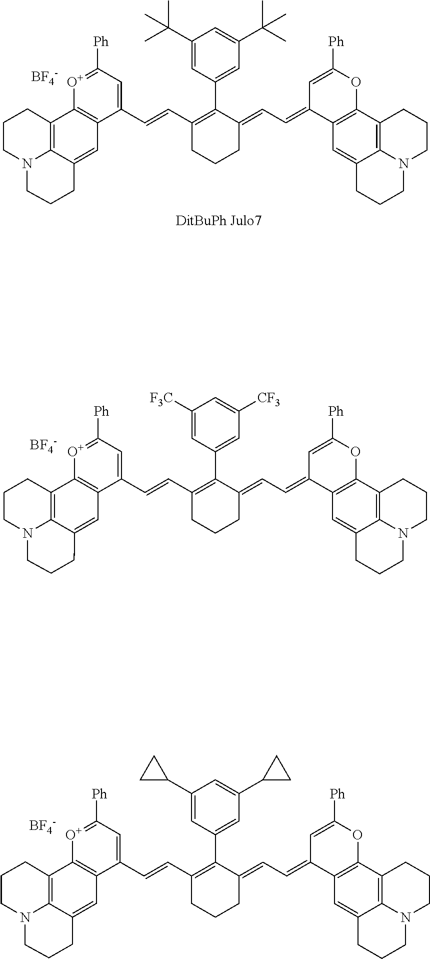 [SMILES: CC(C)(C)c1cc(C2=C(/C=C/c3cc(-c4ccccc4)[o+]c4c5c6c(cc34)CCCN6CCC5)CCC/C2=C\C=C2/C=C(c3ccccc3)Oc3c2cc2c4c3CCCN4CCC2)cc(C(C)(C)C)c1 ; "BF4-" ; FC(F)(F)c1cc(C2=C(/C=C/c3cc(-c4ccccc4)[o+]c4c5c6c(cc34)CCCN6CCC5)CCC/C2=C\C=C2/C=C(c3ccccc3)Oc3c2cc2c4c3CCCN4CCC2)cc(C(F)(F)F)c1 ; "BF4-" ; C1=C(c2ccccc2)Oc2c(cc3c4c2CCCN4CCC3)/C1=C/C=C1\CCCC(/C=C/c2cc(-c3ccccc3)[o+]c3c4c5c(cc23)CCCN5CCC4)=C1c1cc(C2CC2)cc(C2CC2)c1 ; "BF4-"]

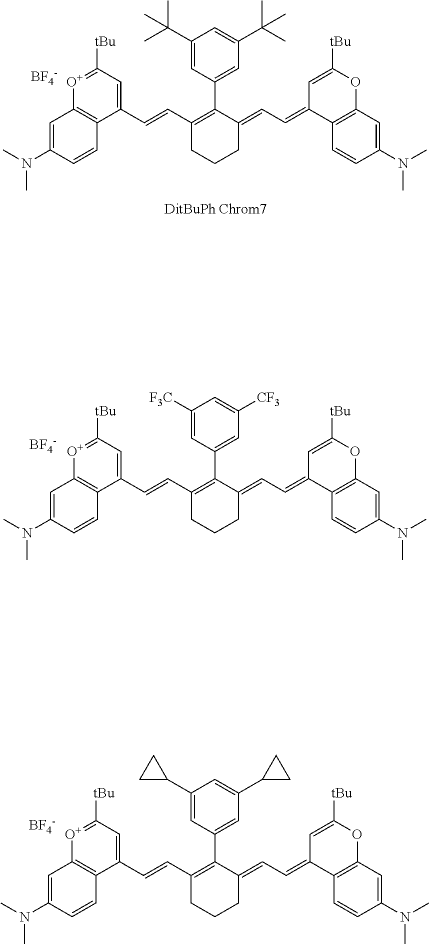

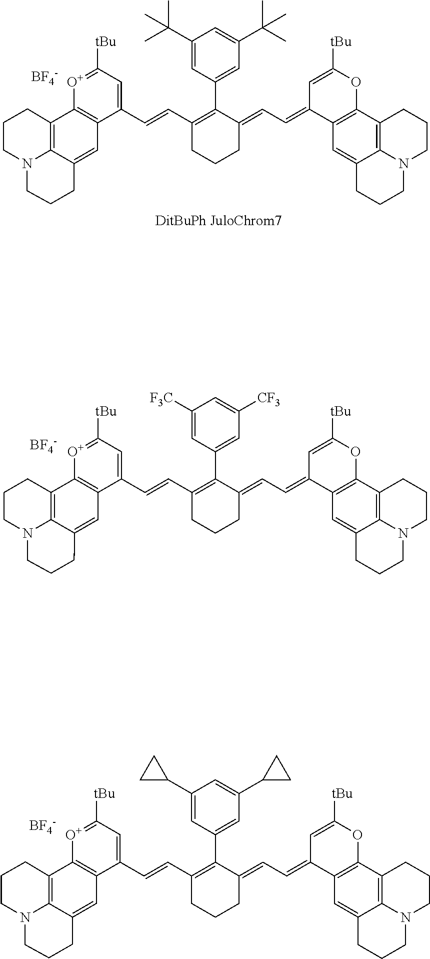 [SMILES: CC(C)(C)C1=C/C(=C\C=C2/CCCC(/C=C/c3cc(C(C)(C)C)[o+]c4c5c6c(cc34)CCCN6CCC5)=C2c2cc(C(C)(C)C)cc(C(C)(C)C)c2)c2cc3c4c(c2O1)CCCN4CCC3 ; "BF4-" ; CC(C)(C)C1=C/C(=C\C=C2/CCCC(/C=C/c3cc(C(C)(C)C)[o+]c4c5c6c(cc34)CCCN6CCC5)=C2c2cc(C(F)(F)F)cc(C(F)(F)F)c2)c2cc3c4c(c2O1)CCCN4CCC3 ; "BF4-" ; CC(C)(C)C1=C/C(=C\C=C2/CCCC(/C=C/c3cc(C(C)(C)C)[o+]c4c5c6c(cc34)CCCN6CCC5)=C2c2cc(C3CC3)cc(C3CC3)c2)c2cc3c4c(c2O1)CCCN4CCC3 ; "BF4-"]

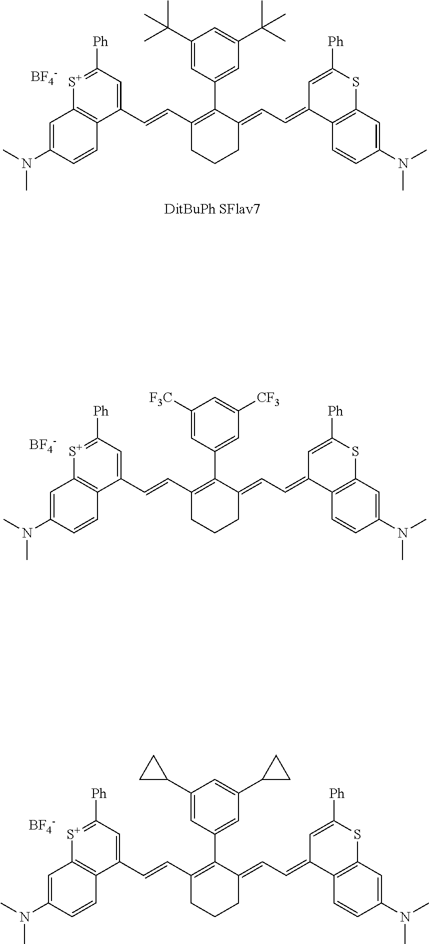

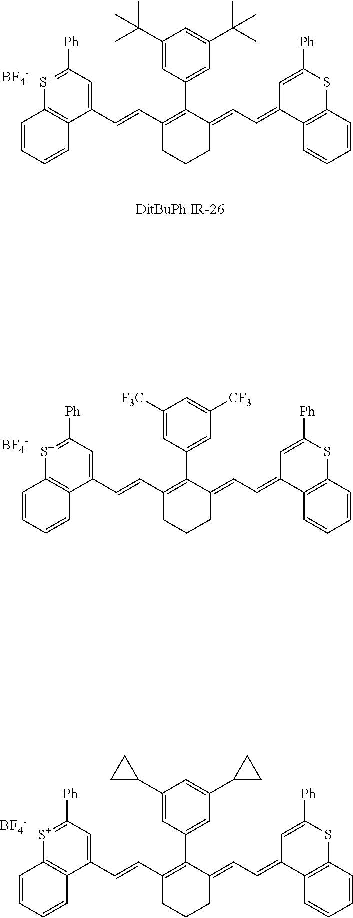 [SMILES: "BF4-" ; CC(C)(C)c1cc(C2=C(/C=C/c3cc(-c4ccccc4)[s+]c4ccccc34)CCC/C2=C\C=C2/C=C(c3ccccc3)Sc3ccccc32)cc(C(C)(C)C)c1 ; "BF4-" ; FC(F)(F)c1cc(C2=C(/C=C/c3cc(-c4ccccc4)[s+]c4ccccc34)CCC/C2=C\C=C2/C=C(c3ccccc3)Sc3ccccc32)cc(C(F)(F)F)c1 ; "BF4-" ; C1=C(c2ccccc2)Sc2ccccc2/C1=C/C=C1\CCCC(/C=C/c2cc(-c3ccccc3)[s+]c3ccccc23)=C1c1cc(C2CC2)cc(C2CC2)c1]

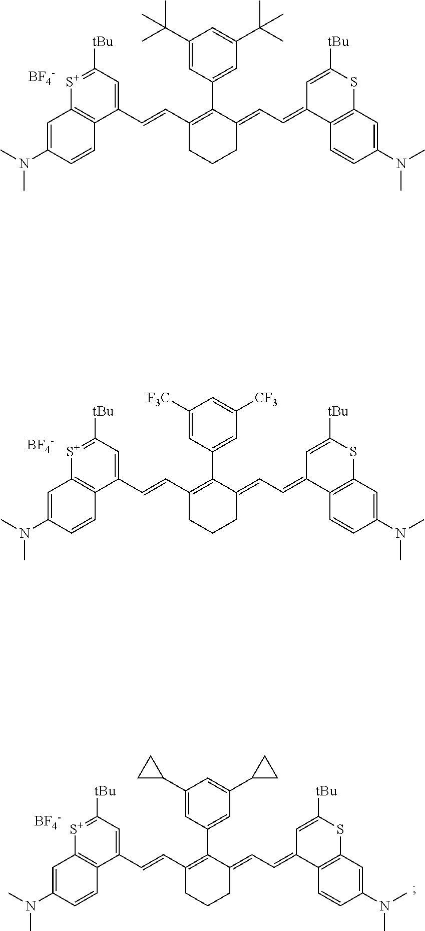

and salts wherein BF₄⁻ is replaced by an anion selected from halide, perchlorate, BAr₄⁻, boron clusters (e.g., a borohydride complex), and TRISPHAT (tetrabutylammonium phosphorus(V) tris(tetrachlorocatecholate)).

In certain aspects, the present disclosure provides pharmaceutical compositions comprising a compound as described herein.

In certain aspects, the present disclosure provides methods of delivering a compound or composition disclosed herein to a living animal, comprising administering the compound or composition to the living animal.

In certain aspects, the present disclosure provides methods of obtaining an image comprising illuminating a compound disclosed herein with excitation light, thereby causing the compound to emit fluorescence; and detecting the fluorescence. In certain embodiments, the image is obtained in vivo. In certain embodiments, the methods further comprise administering the compound to a living animal.

In certain aspects, the present disclosure provides methods of administering a therapy comprising administering a compound or composition disclosed herein, for example to an animal. In certain embodiments the methods further comprise illuminating the compound with excitation light. In certain embodiments, the methods further comprise generating singlet oxygen by illuminating the compound with excitation light.

This disclosure also includes all suitable isotopic variations of a compound of the disclosure. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulfur, fluorine, chlorine, bromine and iodine, such as 2H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Accordingly, recitation of “hydrogen” or “H” should be understood to encompass ¹H (protium), ²H (deuterium), and ³H (tritium) unless otherwise specified. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Such variants may also have advantageous optical properties arising, for example, from changes to vibrational modes due to the heavier isotope. Isotopic variations of a compound of the invention can generally be prepared by conventional procedures known by a person skilled in the art such as by the illustrative methods or by the preparations described in the examples hereafter using appropriate isotopic variations of suitable reagents.

### Small Molecule SWIR Chromophores

SWIR small molecule chromophores are generally characterized by a narrow gap between the highest occupied molecular orbital (IIOMO) and lowest unoccupied molecular orbital (LUMO). As the HOMO-LUMO gap of chromophores decreases, their reactivity increases. Thus, stability of SWIR chromophores is typically a more significant challenge than for NIR chromophores. Another consequence of the small energy difference between the ground state and excited state is that there are often many non-emissive pathways which can facilitate relaxation back to the ground state, resulting in decreased quantum yields of fluorescence (Φ_{F}). For SWIR photodynamic therapy applications, the triplet energies of the photosensitizers need to be high enough to sensitize oxygen (23 kcal/mol).

The present disclosure provides SWIR-active small molecules with improved properties for use in optical imaging, photothermal therapy, and photodynamic therapy.

### General Fluorophore Syntheses and Nomenclature

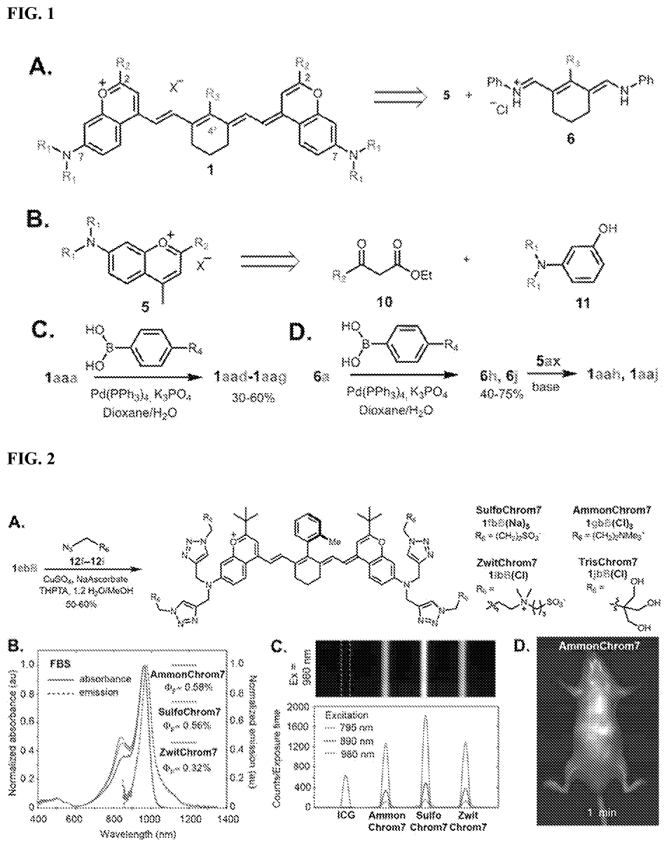

In certain embodiments, chromenylium polymethine fluorophores may be modified at the 7-, 2- and 4′-positions designated R⁵/R⁶/R⁷/R⁸, R³/R⁴, and R^{a}, respectively. In certain embodiments, dyes of the disclosure may have a structure as shown in FIG. 1A. The fluorophores are generally classified as flavylium or chromenylium dyes depending on respective aryl or alkyl functionality at the 2-position. In certain embodiments, polymethine dyes (e.g., 1) are prepared via combining two equivalents of heterocycle (5) with 1 equivalent of a linker (e.g., 6).

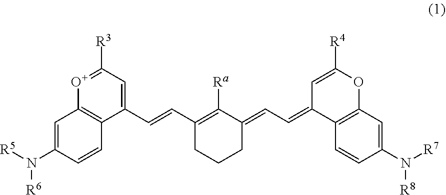

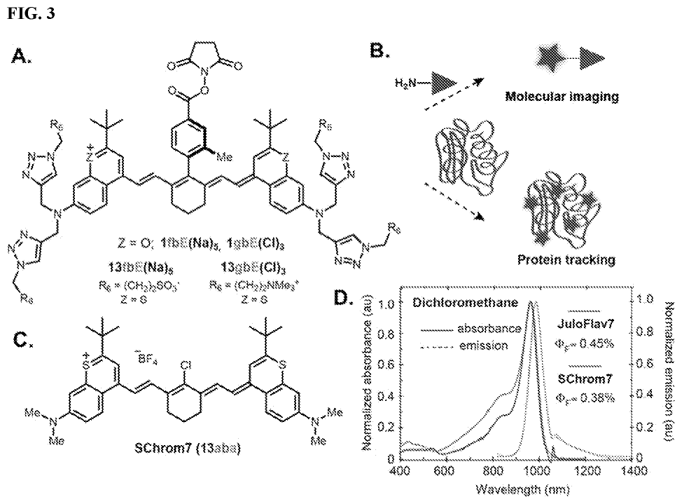

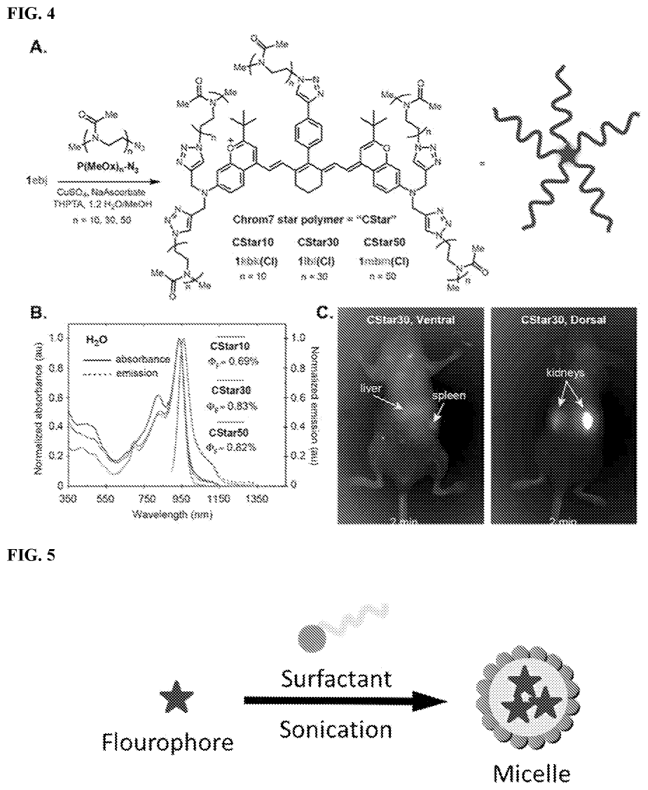

In certain embodiments of the disclosure, heptamethine dyes comprising a cyclohexyl moiety on the polymethine chain (e.g., 1), which include linker 6 (FIG. 1A) are described. In some embodiments, the flavylium/chromenylium heterocycles can be obtained from a β-ketoester (e.g., 10) and a 3-aminophenol (e.g., 11) starting materials that can undergo a Mentzer pyrone synthesis followed by addition of MeMgBr (FIG. 1B). In certain such embodiments, modifying the 7-position includes different aminophenols and the 2-position is dictated by the β-ketoester substitution. The 4′ position of 1 can be modified through Suzuki chemistry either on fluorophore 1 (FIG. 3C) or on linker 6a, which can then be combined with heterocycle (5) to produce the desired heptamethine dye (FIG. 3D). The counterion can be readily exchanged after the dye is prepared. Exemplary embodiments of various R⁵/R⁶/R⁷/R⁸, R³/R⁴, and R^{a} substituents are found in Table 1, below. For example, Flav7=1aaa(ClO₄⁻) where the first a designates Me as R⁵, R⁶, R⁷, and R⁸ off the 7-amino groups, the second a designates Ph as R³ and R⁴ at the 2-position, and the third a designates Cl as R^{a} at the 4′ position. In certain embodiments, 1aaa(ClO₄⁻) can be further modified by reactivity at the Cl atom, as exemplified in FIG. 1C.

In certain embodiments, each of R⁵, R⁶, R⁷, and R⁸ is independently selected from:
alkyl, alkoxyl,

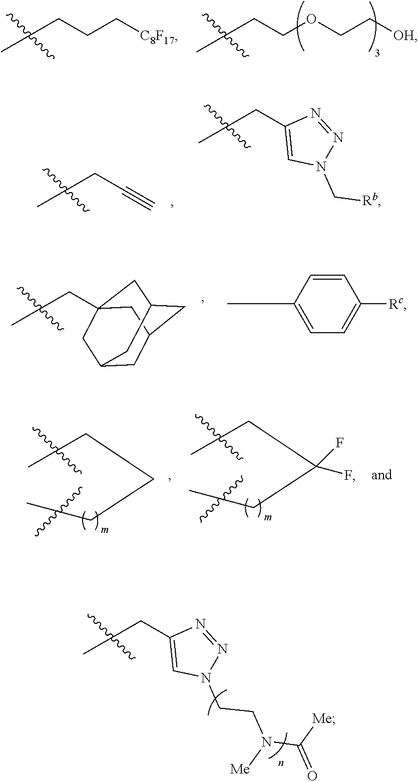

or R⁵ and
R⁶ and/or R⁷ and R⁸, together with the nitrogen to which they are attached, combine to form a heteroaryl or heterocyclyl;
wherein:
m is selected from 1, 2, and 3;
n is selected from 10, 30, 50, 100, and 250;
R^{b} is selected from —(CH₂)₂SO₃, —(CH₂)₂NMe₃⁻, —C(CH₂OH)₃, and

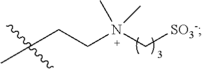

and
R^{c} is selected from H, alkyne, and —CH₂N₃.

In certain embodiments, R³ and R⁴ may each independently be selected from alkyl, alkoxyl,

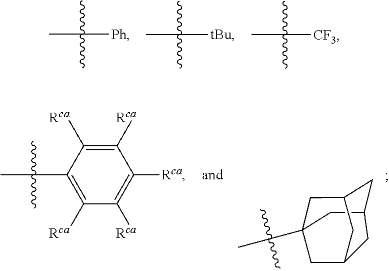

wherein R^{ca} is selected from H, alkoxyl, alkyne, alkyl, halo, haloalkyl, azido, and lower alkylamine.

In certain embodiments, R^{a} may be selected from halo,

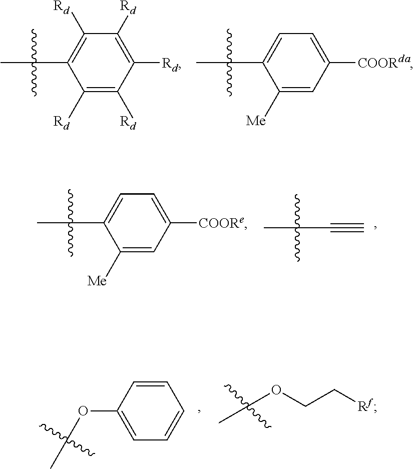

wherein:
R^{d} is selected from H, alkoxyl, alkyne, alkyl, halo, haloalkyl, azido, and lower alkylamine;
R^{da} is selected from H,

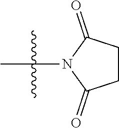 [SMILES: *N1C(=O)CCC1=O]

and tert-butyl·R^{e} is selected from H, —COOH, —(CH₂)₂COOH, —OH, —CH₂N₃,

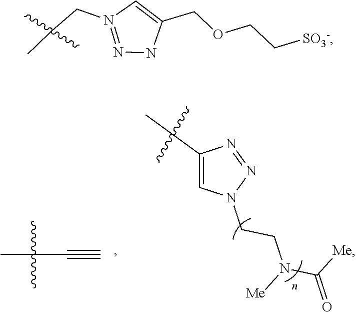

—CF₃, —F, -Me, —OMe, and —NEt₂;
R^{f} is selected from C₃H₇, C₈F₁₇, (CH₂)₃C(O)OCH₃, (CH₂)₃Cl,

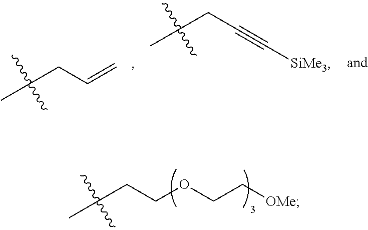

and
n is selected from 10, 30, 50, 100, and 250.

### Water-Soluble Variants of Heptamethine Dyes

In certain embodiments, methods of the disclosure include the following: First, we prepared 1dah, with four tetraethylene glycol moieties and an azide conjugation handle at the 4′ position. In water, 1dah was fully aggregated, although some monomer was observed in 1:1 MeOH/H₂O. Changes to the ratio of monomer: aggregate in 1:1 MeOH/H₂O were observed by adding a sulfonate (1dai) or appending an ortho-methyl group to the 4′-aryl ring to decrease planarity (1daB). Unfortunately, all these fluorophores remained aggregated in water. Next, we combined the benefits of 1dai and 1daB and the less aggregation prone chromenylium scaffold (R₂=tBu). Tetraalkyne-containing chromenylium heptamethine dye 1ebB was prepared via synthetic protocols provided herein and underwent click chemistry with azides 12f-12h to yield 1fbB (Na) 3 “SulfoChrom7” 1gbB(Cl)₅ “Ammon-Chrom7”, 1hbB(Cl) “ZwitChrom7”, and libB(Cl) “TrisChrom7” (FIG. 2A). All 4 fluorophores displayed some monomer in water and when placed in serum, the multiply charged dyes were primarily monomeric (FIG. 2B), suggesting that they bind with protein in a similar manner as ICG and other sulfonated NIR/SWIR dyes. A comparative brightness study in FBS between ICG, AmmonChrom7, SulfoChrom7, and ZwitChrom7 shows all three Chrom7 dyes to be superior SWIR imaging agents to ICG (FIG. 2C) and to have excellent performance in vivo (FIG. 2D).

Molecular Imaging with Water-Soluble Chromenylium Fluorophores

We will prepare functionalizable variants of the fluorophores for targeting different tissues, organs, and cell types. Our previous work determined that the ortho-methyl phenyl substitution at the 4′ position may reduce aggregation and thus we require dye 1ebC which retains the aggregation decreasing tBu groups at the 2-position and ortho-methyl phenyl group at the 4′-position but has alkyne click handles at the 7-position and an acid functional handle at the 4′ position. We have prepared 1ebC from linker 6C. Ultimately acid-functionalized water soluble dyes such as 1fbC(Na)₃ or 1gbC(Cl)₅ can activated (1fbE(Na)₃ or 1gbE(Cl)₅, FIG. 3A) and combined with amine-functionalized targeting agents, peptides, and proteins for molecular imaging and protein tracking (FIG. 3B).

In certain embodiments, water soluble Chrom7 dyes of the disclosure are suitable for imaging with excitation at 980 nm. In certain embodiments, 1064 nm fluorophores, e.g., JuloFlav7 (1baa), feature the julolidine motif at the 7-position and a phenyl group at the 2-position, which together prevent the addition of charged functionality off the 7-position and removal of aggregation-inducing phenyl groups at the 2-position. Thus, we have explored other strategies to red-shift chromenylium polymethine dyes to arrive at a bright fluorophore that can be further functionalized with water-solubilizing groups. Recent success in this area has produced 13aba, “SChrom7”, which has photophysical properties similar to JuloFlav7 (FIG. 3D). Based on these results, we prepared dyes 13fbC(Na)₃ or 13gbC(Cl)₅ as water-soluble, functionalizable, dyes for excitation at 1064 nm.

### Star Polymers Containing Chromenylium Dyes

Star polymers are polymers with multiple polymeric “arms” that extend from a common core. In certain embodiments, we will use the fluorophore as the core of the star polymer. In certain embodiments, there is one fluorophore per nanostructure and the fluorophore will be surrounded by polymeric arms, sterically preventing aggregation from occurring. The star polymer architecture will allow for functionalization with a defined amount of targeting agents. The length of the polymer arms will directly dictate the diameter of the fluorescent star polymer and allow for tuning of the clearance pathways (renal vs. liver) and thus partially control the serum half-lives of the contrast agents. Additionally, the polymer arms can also be modified to contain a ϕ_{F} enhancing block or an anti-photobleaching block to further improve the properties of the contrast agent.

### Exemplary Syntheses of Dyes of the Invention

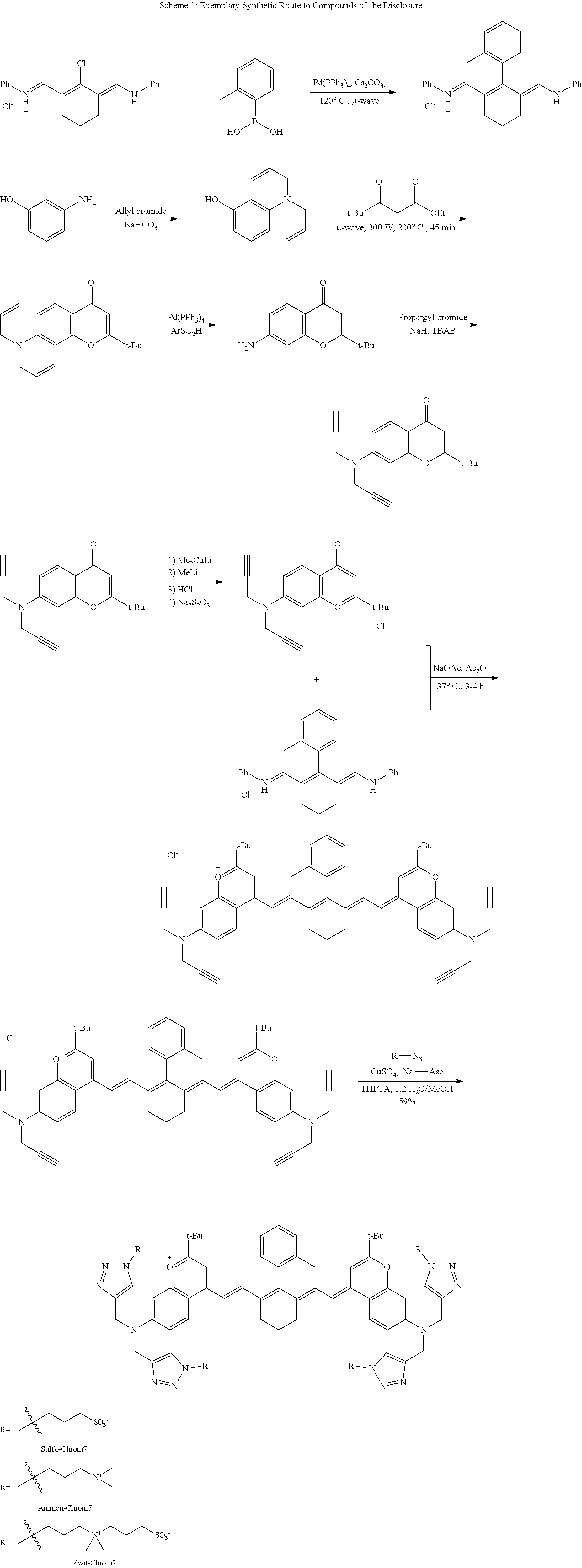

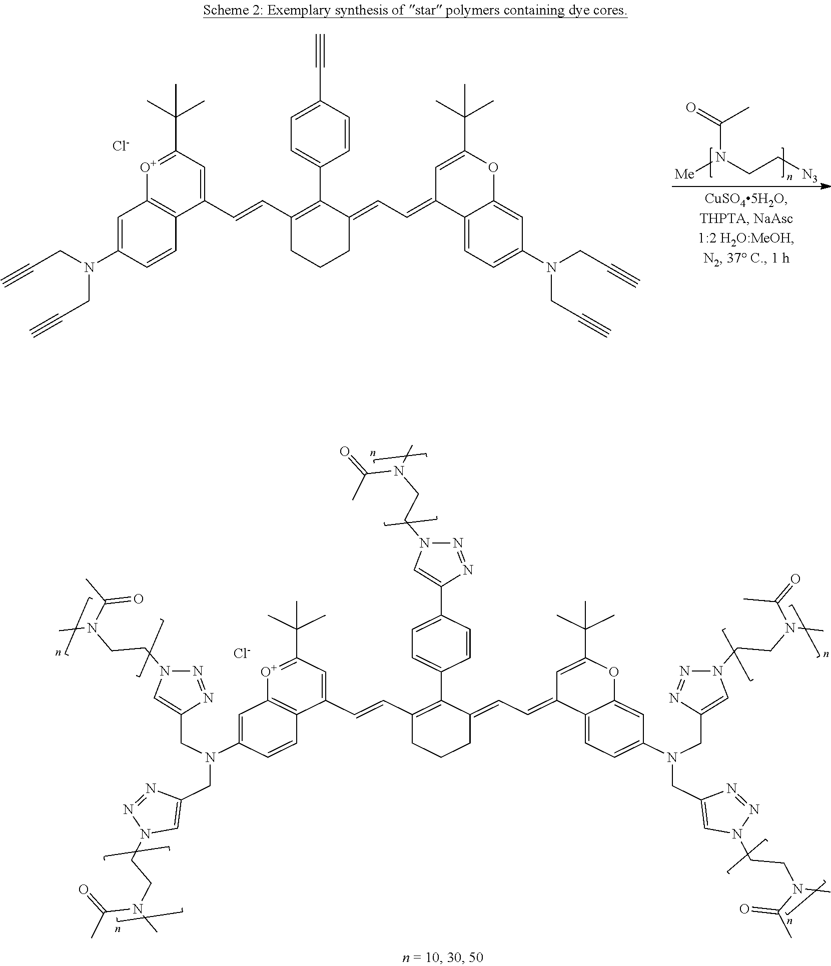

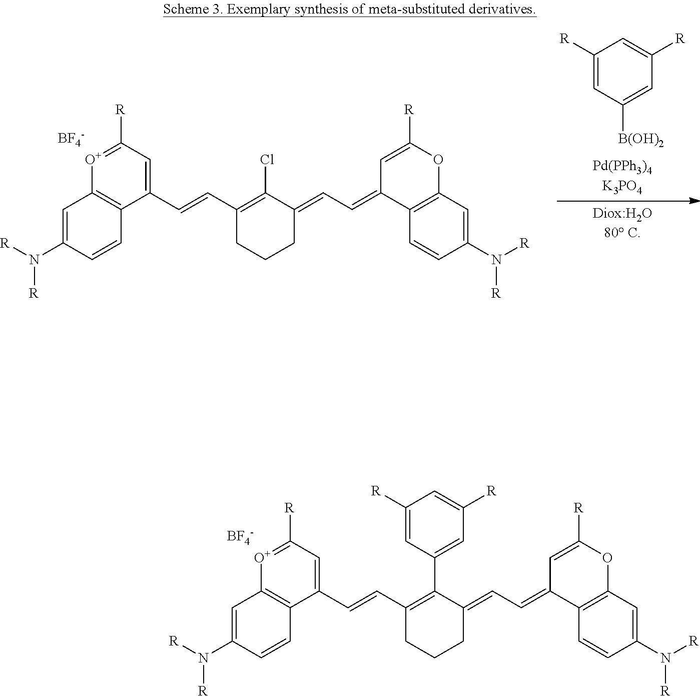

“X dye” (1 equiv.), potassium phosphate tribasic (2 equiv.), palladium tetrakis (0.1 equiv.), and “appropriate meta substituted boronic acid” (5 equiv.) were dissolved in a flame dried microwave vial under N₂ atmosphere. The solution was freeze-pumped-thawed three times and microwaved at 120° C. for 20 minutes. The reaction was then quenched with a 1:1 EtOH:H₂O mixture. The organic material was extracted with DCM. The mixture was then evaporated, loaded onto silica gel, and purified by column chromatography using a 0.5 to 15% DCM to ethanol gradient.

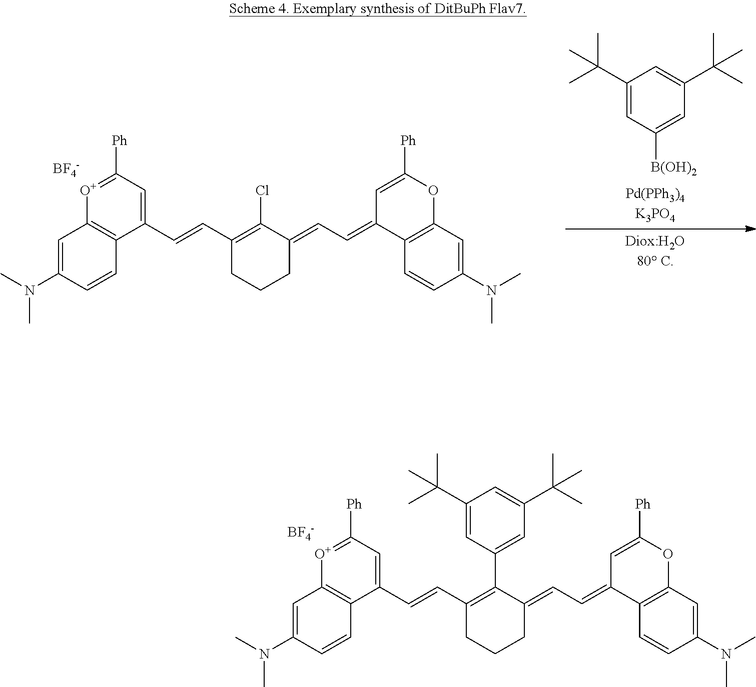

Flav7 (10 mg, 0.015 mmol, 1 equiv.), potassium phosphate tribasic (6.9 mg, 0.03 mmol, 2 equiv.), palladium tetrakis (2.8 mg, 0.0015 mmol, 0.1 equiv.), and 3,5-Di-tert-butylphenylboronic acid (17.5 mg, 0.075 mmol, 5 equiv.) were dissolved in a flame dried microwave vial under N₂ atmosphere. The solution was freeze pumped thawed three times and microwaved at 120° C. for 20 minutes. The reaction was then quenched with a 1:1 EtOH:H₂O mixture. The organic material was extracted with DCM. The mixture was then evaporated, loaded onto silica gel, and purified by column chromatography using a 0.5 to 15% DCM to ethanol gradient.

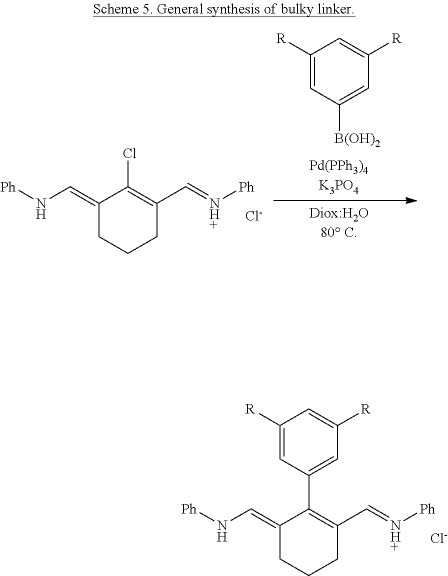

N-((E)-((E)-2-chloro-3-((phenylamino)methylene)cyclohex-1-en-1-yl)methylene)benzenaminium (1 equiv.), potassium phosphate tribasic (2 equiv.), palladium tetrakis (0.1 equiv.), and “appropriate meta substituted boronic acid” (5 equiv.) were dissolved in a flame dried Schlenk flask under N₂ atmosphere. The solution was freeze-pumped-thawed three times and heated at 80° C. for 24 hours. The reaction was then quenched with a 1:1 EtOH:H₂O mixture. The organic material was extracted with DCM. The mixture was then evaporated, loaded onto silica gel, and purified by column chromatography using a 0.5 to 15% DCM to ethanol gradient.

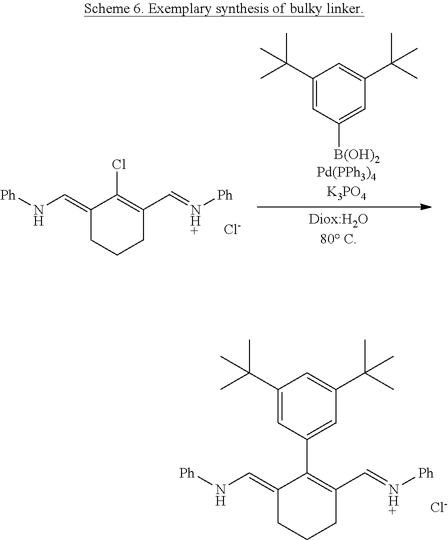 [SMILES: CC(C)(C)c1cc(B(O)O)cc(C(C)(C)C)c1 ; ClC1=C(/C=[NH+]/c2ccccc2)CCC/C1=C\Nc1ccccc1 ; [Cl-] ; CC(C)(C)c1cc(C2=C(/C=[NH+]/c3ccccc3)CCC/C2=C\Nc2ccccc2)cc(C(C)(C)C)c1 ; [Cl-]]

N-((E)-((E)-2-chloro-3-((phenylamino)methylene)cyclohex-1-en-1-yl)methylene)benzenaminium (50 mg, 0.14 mmol, 1 equiv.), potassium phosphate tribasic (64.9 mg, 0.28 mmol, 2 equiv.), palladium tetrakis (16.2 mg, 0.014 mmol, 0.1 equiv.), and 3,5-Di-tert-butylphenylboronic acid (163.9 mg, 0.70 mmol, 5 equiv.) were dissolved in a flame dried Schlenk flask under N₂ atmosphere. The solution was freeze-pumped-thawed three times and heated at 80° C. for 24 hours. The reaction was then quenched with a 1:1 EtOH:H₂O mixture. The organic material was extracted with DCM. The mixture was then evaporated, loaded onto silica gel, and purified by column chromatography using a 0.5 to 15% DCM to ethanol gradient.

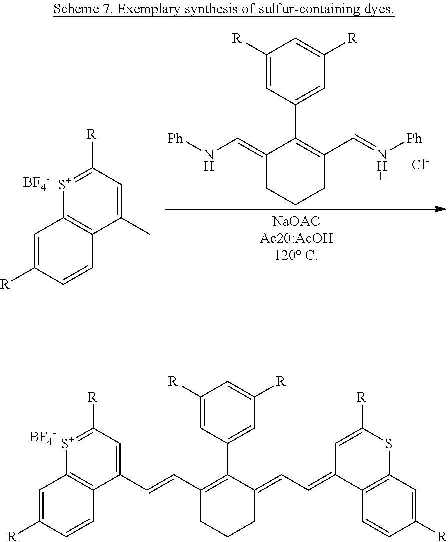

“appropriate heterocycle” (1 equiv.), “appropriate linker” (0.45 equiv.), and sodium acetate (3 equiv.) were dissolved in a 3:2 mixture of n-butanol and toluene (0.2M) in a flame dried Schleck flask under N₂ atmosphere. The solution was freeze-pumped-thawed three times and heated to 100° C. for 15 min. The crude mixture was evaporated, loaded onto silica gel, and purified by column chromatography using a 0.5 to 15% dcm to ethanol gradient.

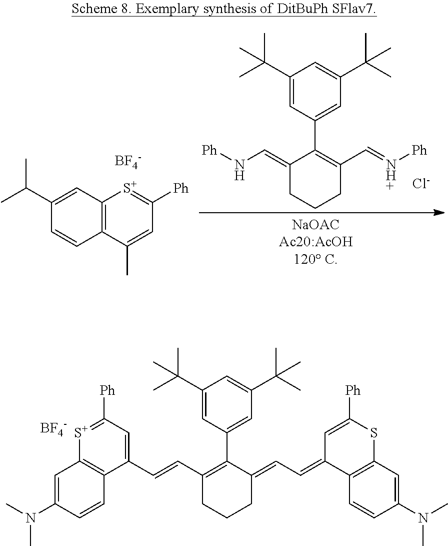

3-dimethylamino flavylium (30.4 mg, 0.087 mmol, 1 equiv.), N-((E)-((E)-3′,5′-di-tert-butyl-6-((phenylamino)methylene)-3,4,5,6-tetrahydro-[1,1′-biphenyl]-2-yl)methylene)benzenaminium chloride (20 mg, 0.039 mmol, 0.45 equiv.), and sodium acetate (22 mg, 0.261 mmol, 3 equiv.) were dissolved in n-butanol (0.29 mL) and toluene (0.14 mL) in a flame dried Schleck flask under N₂ atmosphere. The solution was freeze-pumped-thawed three times and heated to 100° C. for 15 min. The crude mixture was evaporated, loaded onto silica gel, and purified by column chromatography using a 0.5 to 15% dem to ethanol gradient.

Exemplary Optical properties of Compounds of the Disclosure

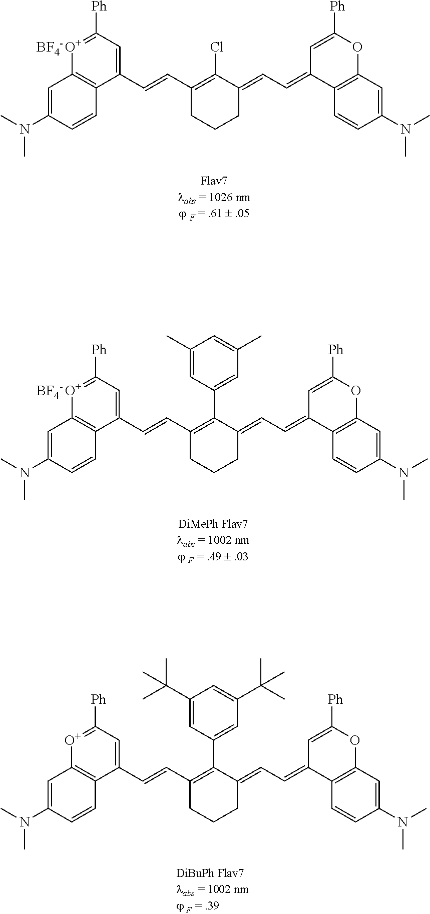

### Photophysical Properties of Exemplary Compounds.

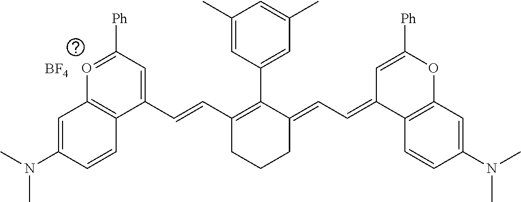

DiMePh Flav7

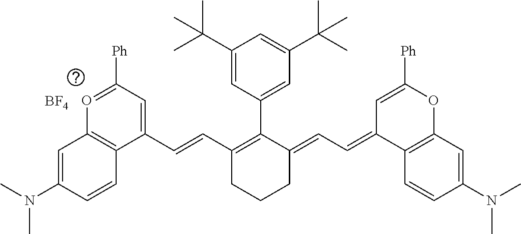

DitBuPh Flav7

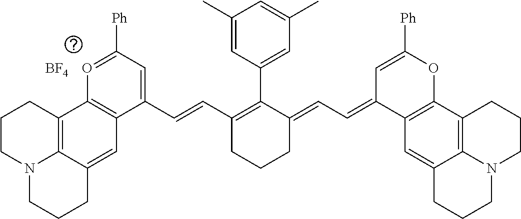

DiMePh Julo7

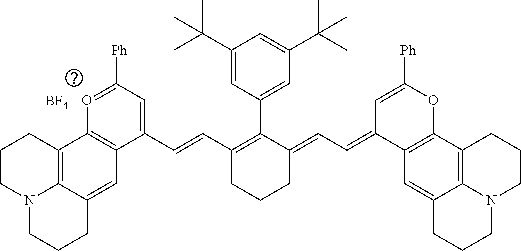

DitBuPh Julo7

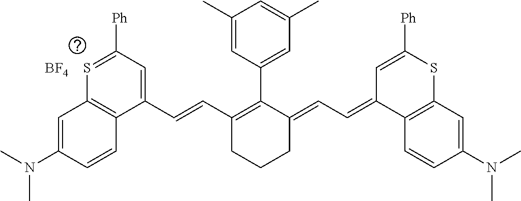

DiMePh SFlav7

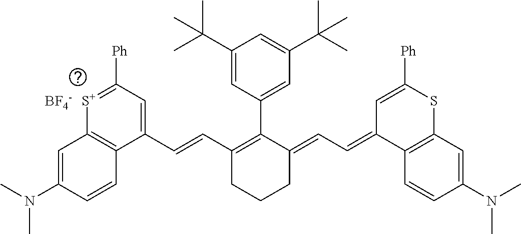

DitBuPh SFlav7

| Compound in a | λ_{max/,cm}/ | ϕ_{F} |
|---|---|---|
| DCM Solution | nm | (%) |
| DiMePh Flav7 | 1004,1026 | 0.49 ± 0.05 |
| DitBuPh Flav 7 | 1004,1026 | 0.39 ± 0.03 |
| DiMePh Julo7 | 1042,1064 | 0.43 ± 0.02 |
| DitBuPh Julo7 | 1042,1062 | 0.31 ± 0.03 |
| DiMePh SFlav7 | 1079,1107 | 0.11 ± 0.01 |
| DitBuPh SFlav7 | 1079,1107 | 0.11 ± 0.02 |

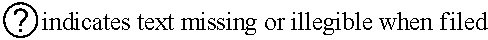

### Synthesis Protocols for Exemplary Micelles

Micelles were synthesized according to the following procedure: 0.1 mg of the desired dye dissolved in 1 mL DMSO and added to 2 mL of a 6 mg/mL solution of 18:0 PEG2000 PE (1,2-distearoyl-sn-glycero-3-phosphoethanolamine-N-[methoxy (polyethylene glycol)-2000] ammonium salt (Avanti Polar Lipids) in a 50 mL falcon tube. The solution was sonicated in a probe sonicator for 3 min on ice at 35% amplitude. The solution was then transferred to a 10 kDa MW cutoff filter (Amicon Ultra-15) and centrifuged at 4000 rpm. Sequential washes with 1×PBS were performed, until the remaining DMSO consisted of <1%.

### Absorbance Measurements of Exemplary Micelles

Micelles of each dye was diluted in 1×PBS and their absorbance traces were obtained in a 2 mm cuvette. Spectra were baseline corrected to the signal at 1300 nm and then normalized. UV-Vis spectra were measured in a JASCO V-770 UV-Vis/NIR spectrophotometer. Reference spectra of unencapsulated monomeric dye Flav7 were taken in DCM.

### Measuring Emissive Properties of Exemplary Micelles

Micelles of each dye were diluted to matching optical density in 1×PBS buffer and transferred to a dram vial. The resulting solutions were transferred to capillary tubes, sealed, then imaged by exciting with 974 nm excitation light and 2 ms exposure, with the appropriate long pass filter attached to the camera. Maximum intensity was plotted in excel. The camera setup is as follows: An InGaAs Camera (Allied Vision Goldeye G-032 Cool TEC2) camera was fitted with a C-mount camera lens (Kowa LM35HC-SW) and emission filters and mounted vertically above an imaging workspace. The camera used a sensor temperature set point of . . . 30° C. The “785” laser (LUMICS, LU0785DLU250-S70AN03, specified to an error of #10 nm) output was coupled cube via a 600 nm core fiber-optic bundle (Lumics, LU_LWL0600_0720_220DIA1). The output from the fiber was fixed in an excitation cube (Thorlabs KCBIE), reflected off of a mirror (Thorlabs BBE1-E03), and passed through a positive achromat (Thorlabs AC254-050-AB-ML), 1,100 nm short-pass filters (Edmund Optics #84-768) and an engineered diffuser (Thorlabs ED1-S20-MD) to provide uniform illumination over the working area. The excitation flux was measured over the area of interest with a digital optical power and energy meter (Thorlabs PM100D). Camera and lasers were externally controlled and synchronized by delivering trigger pulses of 5 V Transistor-Transistor Logic to the laser drivers and camera using a programmable trigger controller with pulses generated with an Atmel Atmega328 micro-controller unit and programmed using Arduino Nano Rev 3 MCU (A000005) in the Arduino integrated development environment (IDE). Acquired imaging data is then transferred to the PC via a Gigabit Ethernet interface. For image acquisition, the toolbox of MATLAB programming environment was used in combination with a MATLAB script (CCDA V3, https://gitlab.com/brunslab/ccda) to preview and collect the required image data in 14-bit depth.

### Absorption Measurements of Exemplary Micelles.

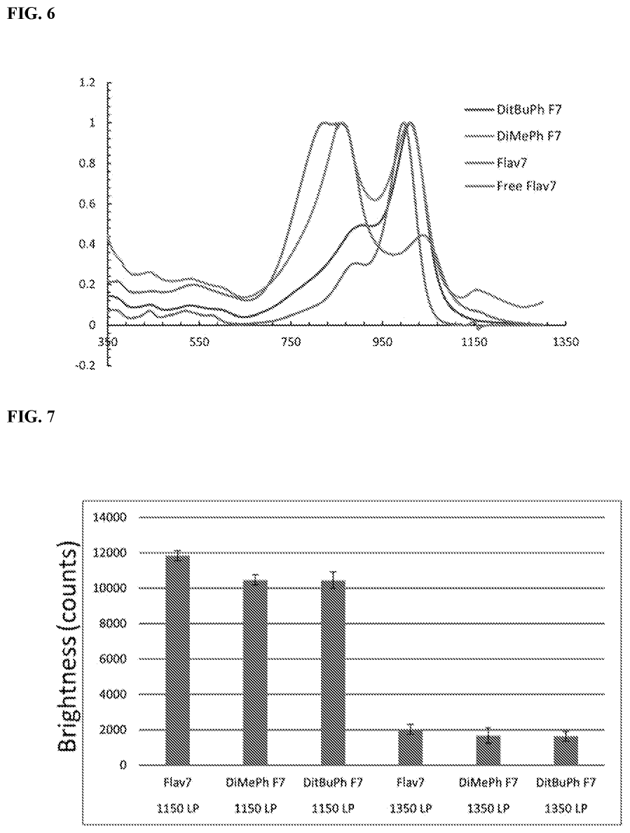

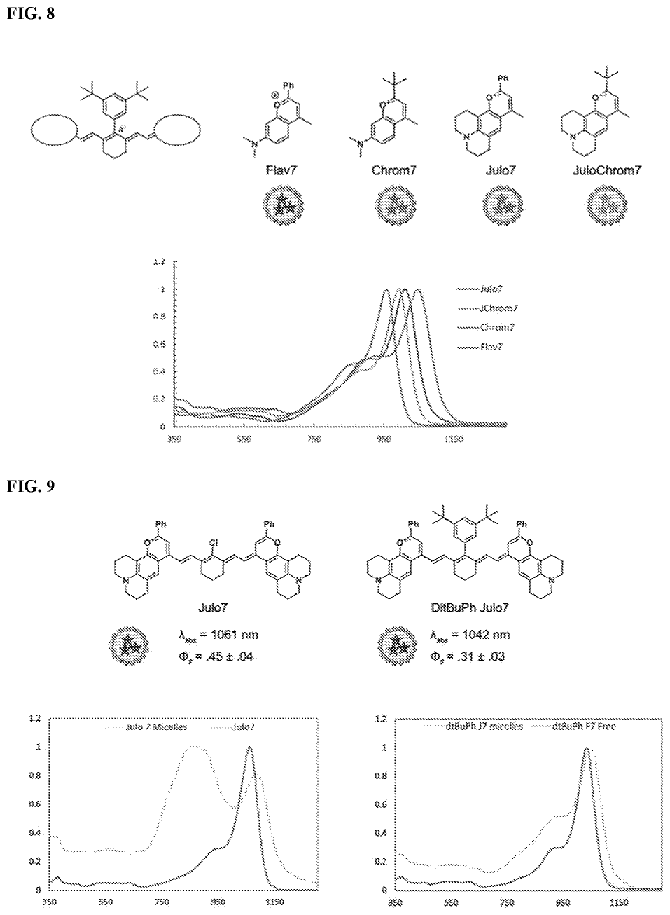

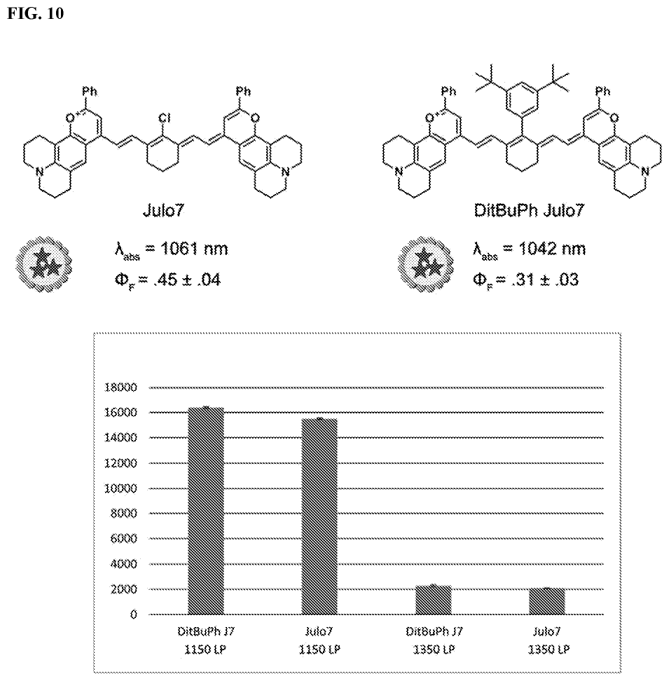

The dyes were encapsulated in micelles according to the procedure described in FIG. 5 and their UV-Vis absorption was measured according to the procedure described in FIG. 6.

Aggregation Experiments with Exemplary Dyes

Aggregation of the dyes was induced as follows. To a 0.1 mM solution of dye in MeOH was added a 0.9% NaCl D20 solution to achieve a 30% MeOH:D₂O solution. The solution was then shaken by hand and its UV-Vis absorption was measured in 0.2 mm slide cuvettes as described in FIG. 6. Reference spectra of monomeric dye were taken in DCM.

Additional synthetic schemes and disclosure relevant to the dyes disclosed herein may be found in U.S. Patent Publication Nos. US2020/0140404A1 and US2021/0363124A1, each of which is incorporated by reference as if fully set forth herein.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

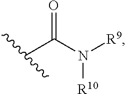

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

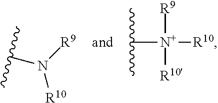

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

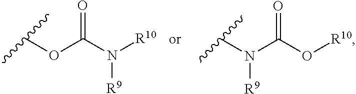

wherein R⁹ and R¹⁰ independently represent hydrogen or a hydrocarbyl group.

The term “carbocyclylalkyl”, as used herein, refers to an alkyl group substituted with a carbocycle group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

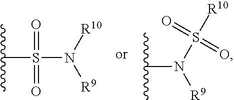

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

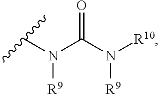

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I or Formula II. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I or Formula II are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I or Formula II for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or Formula II or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of Formula I or Formula II). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I or Formula II. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

The term “deuterium-containing compound of general formula (I) or (II)” and “tritium-containing compound of general formula (I) or (II)” are defined as a compound of general formula (I) or (II), in which one or more hydrogen atom(s) is/are replaced by one or more deuterium and/or tritium atom(s) and in which the abundance of deuterium or tritium at each deuterated or triterated position of the compound of general formula (I) or (II) is higher than the natural abundance of deuterium, which is about 0.015%, or tritium, which is about 1×10⁻¹⁸%. Particularly, in a deuterium-containing or tritium-containing compound of general formula (I) or (II), the abundance of deuterium or tritium at each deuterated or triterated position of the compound of general formula (I) or (II) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium or tritium at each deuterated or triterated position is independent of the abundance of deuterium or tritium at other deuterated or triterated position(s).

The selective incorporation of one or more deuterium atom(s) into a compound of general formula (I) or (II) may alter the physicochemical properties (such as for example acidity [C. L. Perrin, et al., J. Am. Chem. Soc., 2007, 129, 4490; A. Streitwieser et al., J. Am. Chem. Soc., 1963, 85, 2759;], basicity [C. L. Perrin et al., J. Am. Chem. Soc., 2005, 127, 9641; C. L. Perrin, et al., J. Am. Chem. Soc., 2003, 125, 15008; C. L. Perrin in Advances in Physical Organic Chemistry, 44, 144], lipophilicity [B. Testa et al., Int. J. Pharm., 1984, 19 (3), 271]), and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102; D. J. Kushner et al., Can. J. Physiol. Pharmacol., 1999, 77, 79). These changes in the exposure to parent drug and metabolites can have important consequences with respect to the pharmacodynamics, tolerability and efficacy of a deuterium-containing compound of general formula (I) or (II). In some cases deuterium substitution reduces or eliminates the formation of an undesired or toxic metabolite and enhances the formation of a desired metabolite (e.g., Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g., Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g., lower number of doses or lower dosage to achieve the desired effect) and/or may produce lower metabolite loads.

In some embodiments, deuterated or triturated compounds of the disclosure may have other advantageous features, such as an increased quantum yield. This may result from alterations to the available molecular vibrational modes that can reduced coupling between optical and vibrational transitions, thus reducing the rate of intersystem conversion.

A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA. Laboratories Inc., Andover, MA, USA; and CombiPhos Catalysts, Inc., Princeton, NJ, USA. Further information on the state of the art with respect to deuterium-hydrogen exchange is given for example in Hanzlik et al., J. Org. Chem. 55, 3992-3997, 1990; R. P. Hanzlik et al., Biochem. Biophys. Res. Commun. 160, 844, 1989; P. J. Reider et al., J. Org. Chem. 52, 3326-3334, 1987; M. Jarman et al., Carcinogenesis 16 (4), 683-688, 1995; J. Atzrodt et al., Angew. Chem., Int. Ed. 2007, 46, 7744; K. Matoishi et al., Chem. Commun. 2000, 1519-1520; K. Kassahun et al., WO2012/112363.

The term “excitation light” as used herein refers to electromagnetic radiation, i.e., light, of correct energy to “excite,” or induce the transition of a valence electron of the molecule upon which the excitation light is incident from a “ground state” to an “excited state.” Generally herein, the molecules upon which the excitation light are supposed to act are any of the compounds disclosed herein.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient (e.g., dye) which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds (e.g., dyes) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid-addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXAMPLES

The invention now being generally described, it will be more readily understood by reference to the following examples, which are included merely for purposes of illustration of certain aspects and embodiments of the present invention, and are not intended to limit the invention.

### Example 1: Preparation and Evaluation of Exemplary Dyes

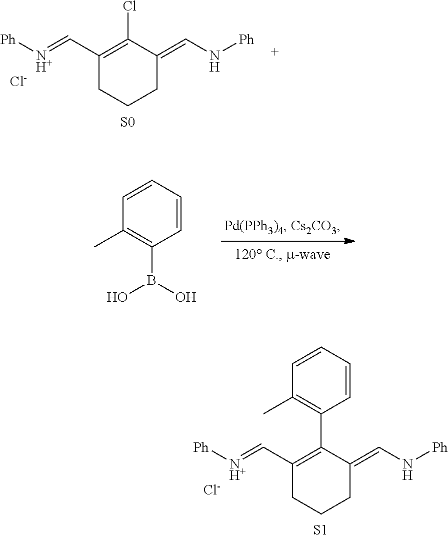

N-((E)-((E)-2′-methyl-6-((phenylamino)methylene)-3,4,5,6-tetrahydro-[1,1′-biphenyl]-2-yl)methylene)benzenaminium chloride (S1): S0 (881 mg, 2.45 mmol) and 2-phenylboronic acid (500 mg, 3.68 mmol), Pd(PPh₃)₄ (283 mg, 0.25 mmol) and Cs₂CO₃ (2.40 g, 7.36 mmol) were weighed into a 35 mL microwave reaction vessel. Dry DME (5 mL) was added followed by three freeze-pump-thaw cycles to fill in N₂. Reaction was carried out at 120° C. under 300 W microwave irradiation for 1 h. The red orange supernatant was diluted in H₂O (50 mL) and extracted with CH₂Cl₂ (3×50 mL). The organic phase was dried (Na₂SO₄), concentrated, separated by column chromatography (1:10-1:7.5 EtOAc/hexanes) and acidified by dry HCl in MeOH to give S1 as a dark red solid (495 mg, 86%). Rr in 1:5 EtOAc/hexanes: 0.3. ¹H NMR (400 MHZ, MeOD) δ 7.56-7.38 (m, 4H), 7.33 (t, J=7.8 Hz, 6H), 7.26 (d, J=7.5 Hz, 1H), 7.21-7.16 (m, 2H), 7.01 (d, J=8.4 Hz, 4H), 2.85-2.68 (m, 4II), 2.24 (s, 3II), 2.16-2.05 (m, 2II). ¹³C NMR (101 MIIz, MeOD) δ 171.38, 151.56, 140.74, 138.22, 135.95, 131.72, 131.58, 131.01, 130.78, 127.46, 127.05, 119.73, 119.18, 23.99, 21.42, 19.24.

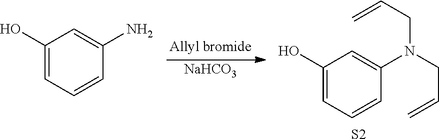

3-(Diallylamino) phenol (S2): 3-Aminophenol (2.5 g, 23 mmol), allyl bromide (6.9 g, 57 mmol) and NaHCO₃ (4.9 g, 46 mmol) was dissolved in 1:1 water/isopropanol mixture (50 mL) followed by two freeze-pump-thaw cycles to fill in N₂. Reaction mixture was stirred for 18 h under 70° C. The solution was concentrate and extracted with CH₂Cl₂ (3×50 mL). The crude product was separated by column chromatography (1:15 EtOAc/hexanes) to give S2 as a yellow thick liquid (2.71 g, 63%). Rf in 1:5 EtOAc/hexanes: 0.3. ¹H NMR (500 MHZ, CD₃CN) δ 6.96 (t, J=8.1 Hz, 1H), 6.57 (s, 1H), 6.23-6.20 (m, 1H), 6.14 (t, J=2.3 Hz, 1H), 6.08 (ddd, J=7.9, 2.2, 0.7 Hz, 1H), 5.91-5.82 (m, 2H), 5.17-5.11 (m, 4H), 3.89 (dt, J=4.7, 1.5 Hz, 4H). ¹³C NMR (126 MHZ, CDCl₃) δ 158.74, 151.08, 135.48, 130.71, 116.09, 105.34, 104.08, 100.22, 53.49.

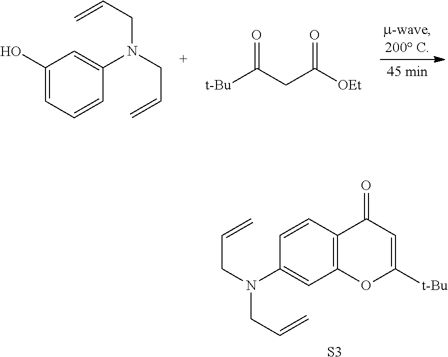

2-(tert-butyl)-7-(diallylamino)-4H-chromen-4-one (S3): S2 (2.71 g, 14.3 mmol) and ethyl 4,4-dimethyl-3-oxopentanoate (7.40 g, 43.0 mmol) was combined in a 10 mL microwave reaction vessel. The reaction was carried out at 120° C. under 300 W microwave irradiation for 45 min, keeping pressure under 200 psi. The dark brown oil was separated by column chromatography (1:4 to 1:3 EtOAc/hexanes) to give S3 as a thick red-brown oil (2.31 g, 54%). Rf in 1:1 EtOAc/hexanes: 0.5. ¹H NMR (500 MHZ, CDCl₃) δ 7.95 (d, J=9.0 Hz, 1H), 6.70 (dd, J=9.0, 2.3 Hz, 1H), 6.49 (d, J=2.3 Hz, 1H), 6.12 (s, 1H), 5.86 (ddd, J=15.0, 9.8, 4.6 Hz, 2H), 5.21 (d, J=10.4 Hz, 2H), 5.17 (dd, J=17.2, 1.1 Hz, 2H), 4.01-3.99 (m, 4H), 1.31 (s, 9H). ¹³C NMR (126 MHz, CDCl₃) δ 178.48, 174.91, 158.81, 152.98, 132.40, 126.66, 116.77, 113.57, 110.89, 106.15, 97.38, 52.77, 36.37, 28.05.

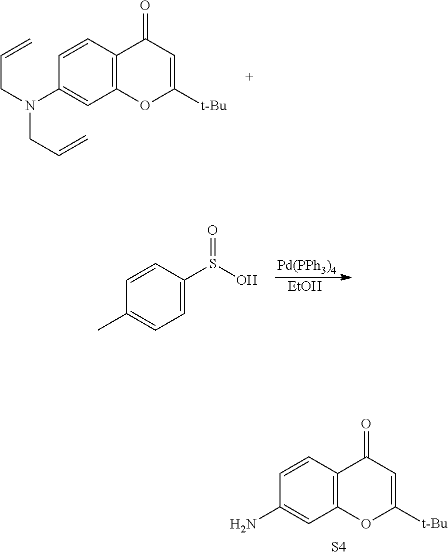

7-amino-2-(tert-butyl)-4H-chromen-4-one (S4): S3 (2.31 g, 7.7 mmol), 4-methylbenzenesulfinic acid (7.24 g, 46.4 mmol) and Pd(PPh₃)₄ (0.45 g, 0.39 mmol) were mixed with ethanol (100 mL) followed by three freeze-pump-thaw cycles to fill in N₂. The reaction was stirred at 40° C. for 21 h. The mixture was concentrated, diluted in 1 M NaOH (60 mL) and extracted with CH₂Cl (3×50 mL). The crude product was separated by column chromatography (2:1 to 1:1 EtOAc/hexanes) to give S4 as a thick red-brown oil (1.53 g, 91%). Rf in 1:1 EtOAc/hexanes: 0.3. ¹H NMR (400 MHZ, CDCl₃) δ 7.92 (d, J=8.6 Hz, 1H), 6.64 (dd, J=8.6, 2.2 Hz, 1H), 6.57 (d, J=2.1 Hz, 1H), 6.13 (s, 1H), 1.29 (s, 9H). ¹³C NMR (101 MHz, CDCl₃) δ 178.45, 175.08, 158.68, 151.99, 127.11, 115.14, 113.55, 106.17, 100.05, 36.36, 27.96.

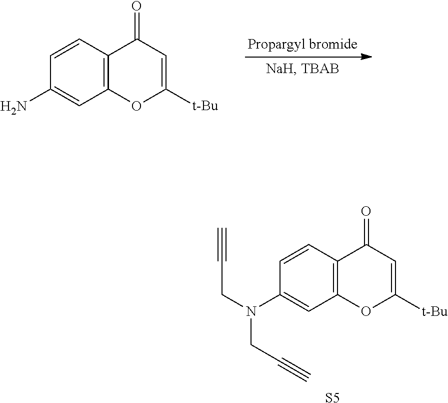

2-(tert-butyl)-7˜ (dipropargylamino)-4H-chromen-4-one (S5): S4 (1.53 g, 7.04 mmol), propargyl bromide (3.9 mL of 80% solution in toluene, 35.2 mmol), tetrabutylammonium bromide (6.81 g, 21.1 mmol) and NaH (2.25 g of 60% dispersion in mineral oil) was weighed into a 500 mL flask. Dry THF (200 mL) was added and the reaction was stirred under N₂ at 45° C. for 3 h. The reaction mixture was quenched by careful addition of MeOH and H₂O, concentrated, diluted in saturated NaHCO₃ (100 mL), and extracted with CH₂Cl (4×50 mL). The organic phase was dried (Na₂SO₄), concentrated and purified by column chromatography (1:3 EtOAc/hexanes) to give S5 as a thick, red brown oil (0.66 g, 32%). Rf in 1:1 EtOAc/hexanes: 0.5. ¹H NMR (500 MHz, CDCl₃) δ 8.04 (d, J=8.9 Hz, 1H), 6.92 (dd, J=9.0, 2.4 Hz, 1H), 6.76 (d, J=2.4 Hz, 1H), 6.15 (s, 1H), 4.21 (d, J=2.2 Hz, 4H), 2.31 (t, J=2.1 Hz, 2H), 1.32 (s, 12H). ¹³C NMR (101 MHz, MeOD) δ 171.38, 151.56, 140.74, 138.22, 135.95, 131.72, 131.58, 131.01, 130.78, 127.46, 127.05, 119.73, 119.18, 23.99, 21.42, 19.24.

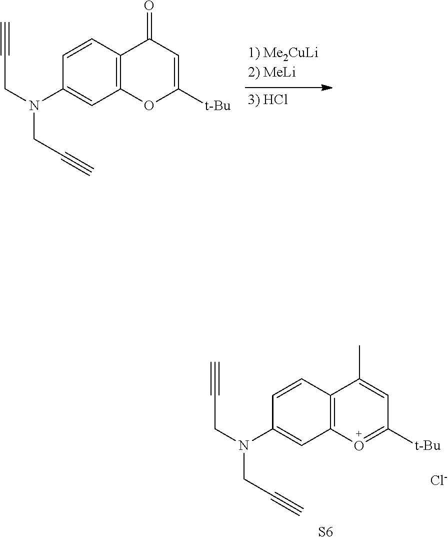

2-(tert-butyl)-7-(di(prop-2-yn-1-yl)amino)-4-methylchromenylium chloride (S6): To a 125 mL flask containing CuI (632 mg, 3.27 mmol) was added dry THF (40 mL) and cooled in dry ice/acetone bath. MeLi (1.6 M in Et₂O, 3.8 mL, 6.0 mmol) was added dropwise and the mixture was stirred for 10 min. A solution S5 (320 mg, 1.09 mmol) in dry THF (10 mL) was transferred to the reaction system dropwise and stirred for another 10 min. MeLi (1.6 M in Et₂O, 6.1 mL) was then added. The reaction mixture was allowed to warm up to room temperature and stirred for another 15 min, followed by quenching by addition of 1:10 HCl (33 mL). The mixture was then concentrated and filtered. The filtrate was extracted with CH₂Cl₂ (4×75 mL). The combined organic phase was further washed with aqueous Na₂S₂O₃ solution (0.3 g/mL, 2×20 mL), dried (Na₂SO₄), concentrated and triturate with Et₂O to give S6 as a brown solid (280 mg, 79%), which was used without further purification. ¹H NMR (500 MHZ, CDCl₃) δ 8.28 (d, J=9.5 Hz, 1H), 7.60 (d, J=9.4 Hz, 1H), 7.39 (s, 1H), 7.18 (d, J=1.6 Hz, 1H), 4.53 (sk, 4H), 3.02 (s, 3H), 2.53 (s, 2H), 1.56 (s, 9H).

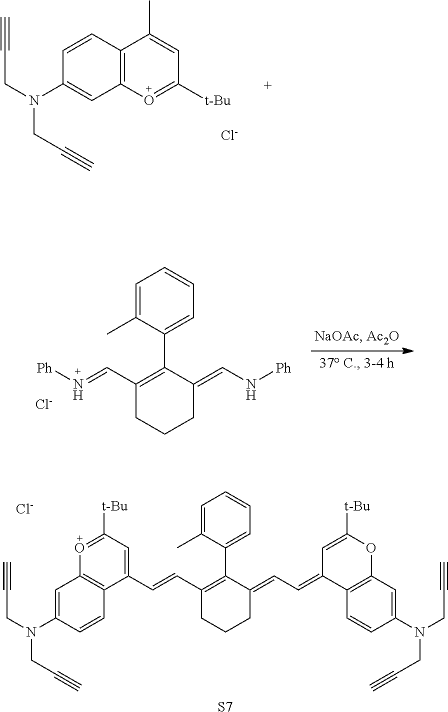

Propargyl-Chrom7 (S7): To a vial containing S1 (96 mg, 0.22 mmol), S6 (191 mg, 0.58 mmol) and NaOAc (142 mg, 1.74 mmol) was added acetic anhydride (12 mL) followed by three cycles of freeze-pump-thaw to fill in N₂. The reaction was stirred for 4.5 h at 37° C. followed by column chromatography (1:80 MeOH/CH₂Cl₂) to give S6 as a dark brown solid (38.6 mg, 22%). 1H NMR (500 MHZ, CD₂Cl₂) δ 7.97 (d, J=9.3 Hz, 2H), 7.43-7.32 (m, 3H), 7.23-7.15 (m, 3H), 7.08 (d, J=9.1 Hz, 2H), 6.89 (d, J=13.7 Hz, 2H), 6.79 (s, 2H), 6.16 (s, 2H), 4.31 (s, 8H), 2.92-2.74 (m, 4H), 2.42 (s, 4H), 2.15 (s, 3H), 2.12-2.02 (m, 2H), 1.22 (s, 18H). ¹³C NMR (126 MHz, CDCl₃) δ 170.68, 161.87, 156.48, 152.02, 146.48, 144.30, 137.70, 135.35, 130.97, 130.21, 128.65, 125.89, 125.66, 120.50, 114.36, 113.71, 110.97, 100.58, 100.12, 78.06, 73.72, 40.87, 36.86, 27.82, 25.77, 21.84, 19.55. HRMS (ESI⁺) calcd 775.4258, found 775.4256 for C₅₅H₅₅N₂O₂⁺ (M⁺).

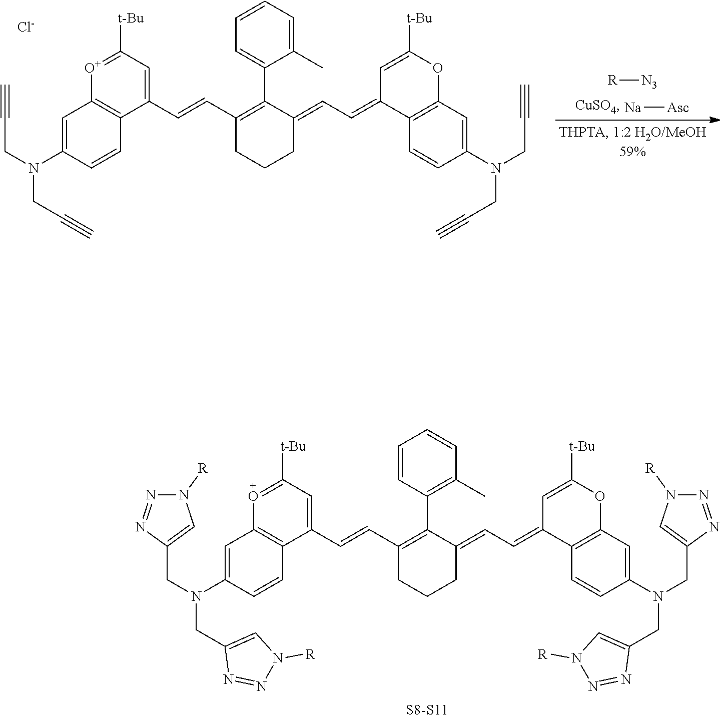

General procedure for CuAAC: S7 (12.5 mg, 0.0154 mmol) was dissolved in MeOH. To this solution was added azide (0.185 mmol), CuSO₄ (12.5 μL of 100 mM solution in H₂O), THPTA (125 μL of 10 mM solution in H₂O) and sodium ascorbate (12.5 mg) and water to afford 1:2 H₂O/MeOH solution unless otherwise noted. The reaction was then filled with N₂ with three freeze-pump-thaw cycles and then stirred for 1 h at 37° C. unless otherwise noted. The reaction was quenched by addition of Na₂EDTA solution (125 μL, 250 mM), concentrated and separated by semi-prep HPLC.

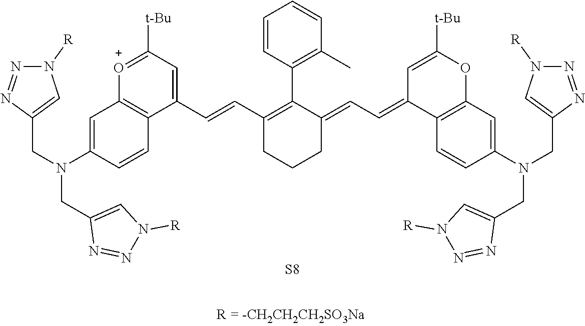

Sulfo-Chrom7 (S8): Following the general procedure, S7 was reacted with sodium 3-azidopropylsulfonate (34.5 mg, 0.185 mmol) to afford S8 (13.6 mg, 59%) as a dark brown solid. ¹H NMR (500 MHZ, CDCl₃) δ 8.04 (s, 4H), 7.97 (d, J=9.1 Hz, 2H), 7.49-7.37 (m, 2H), 7.34 (s, 1H), 7.30-6.90 (m, 5H), 6.83 (s, 3H), 6.03 (s, 2H), 4.57 (t, J=6.9 Hz, 8H), 2.98-2.53 (m, 12H), 2.41-2.25 (m, 8H), 2.08 (s, 3H), 2.03-1.83 (m, 2H), 1.15 (s, 18H).

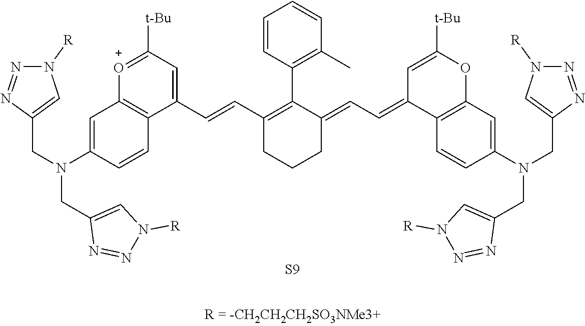

Ammon-Chrom7 (S9): Following the general procedure, S7 was reacted with sodium 3-azido-N,N,N-trimethylpropan-1-aminium trifluoromethylsulfonate (34.5 mg, 0.185 mmol) and purified by semi-prep IIPLC with 50% saturated NaCl flush after sample loading to afford the chloride salt of S9 (13.8 mg, 59%) as a dark brown solid. ¹H NMR (500 MHZ, MeOD) & 8.06 (s, 4H), 8.03 (d, J=9.5 Hz, 2H), 7.49-7.36 (m, 3H), 7.23 (d, J=9.3 Hz, 2H), 7.21-7.11 (m, 3H), 6.97 (s, 1H), 6.95 (d, J=1.9 Hz, 3H), 6.14 (s, 2H), 4.89 (s, 8H), 4.52 (t, J=6.7 Hz, 8H), 3.46-3.37 (m, 8H), 3.14 (s, 36H), 2.92-2.74 (m, 4H), 2.50-2.36 (m, 8H), 2.16 (s, 3H), 2.12-2.02 (m, 2H), 1.20 (s, 18H). HRMS (ESI⁺) calcd 269.5885, found 269.5892 for C₇₉H₁₁₅N₁₈O₂⁵⁺ (M⁵⁺).

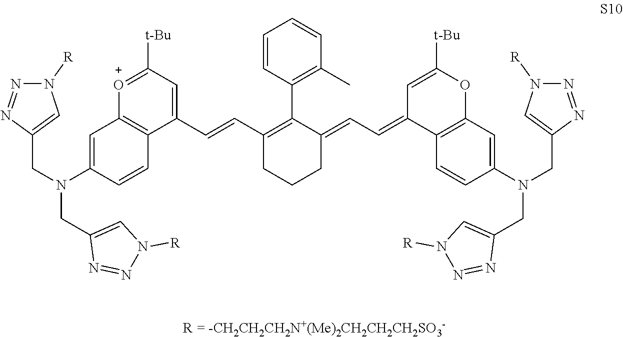

Zwit-Chrom7 (S10): Following the general procedure, S7 was reacted with 3-((3-azidopropyl)dimethylammonio)propane-1-sulfonate (46.3 mg, 0.185 mmol) to afford S10 (16.6 mg, 59%) as a dark brown solid. 1H NMR (500 MHZ, MeOD) δ 8.12 (s, 4H), 8.07 (t, J=6.3 Hz, 1H), 8.04-7.93 (m, 2H), 7.50-7.39 (m, 2H), 7.39-7.31 (m, 1H), 7.31-7.17 (m, 2H), 7.13-6.97 (m, 3H), 6.94-6.77 (m, 3H), 6.05 (mf, 2H), 4.80 (s, 8H), 4.53 (s, 8H), 3.62-3.51 (m, 8H), 3.47-3.37 (m, 8H), 3.13 (s, 24H), 2.94-2.73 (m, 12H), 2.44 (s, 8H), 2.13 (s, 8H), 2.09-2.00 (m, 5H), 1.15 (s, 18H). HRMS (ESI⁺) calcd 888.9382, found 888.9436 for C₈₇H₁₂₈N₁₈O₁₄S4²⁺ ([M+H⁺]²⁺).

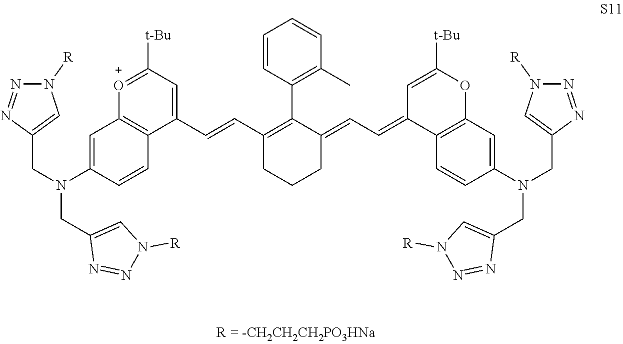

Phospho-Chrom7 (S11): Following the general procedure, S7 (15 mg, 0.018 mmol) was reacted with 3-azidopropylphosphonic acid (37 mg, 0.22 mmol) and NaHCO₃ (19 mg, 0.22 mmol) with CuSO₄, THPTA and sodium ascorbate in 1:2 MeOH/HEPES buffer (150 mmol, pH 7.5) at 42° C. for 6 h to afford S11 as a dark brown solid (5.5 mg, 20%). LRMS (ESI⁺) calcd 1435.5, found 1435.4 for C₆₇H₈₇N₁₄O₁₄P₄⁺ (M⁺).

### Example 2: Formulation and Aggregation Studies

In certain embodiments, dyes of the disclosure include compounds of the following structures:

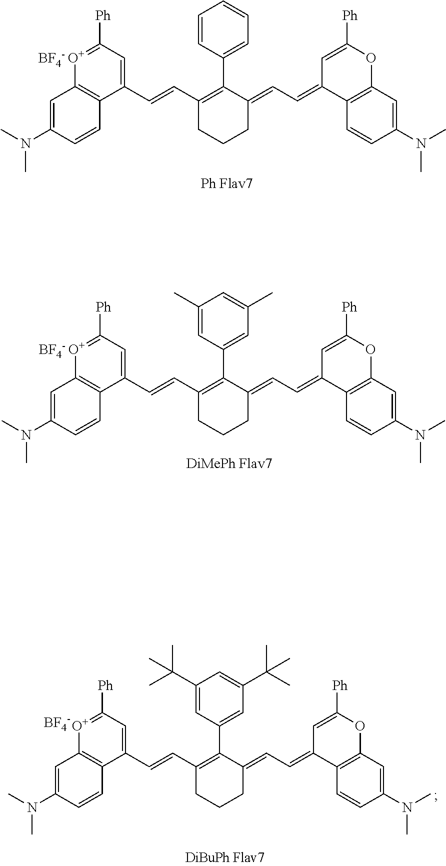

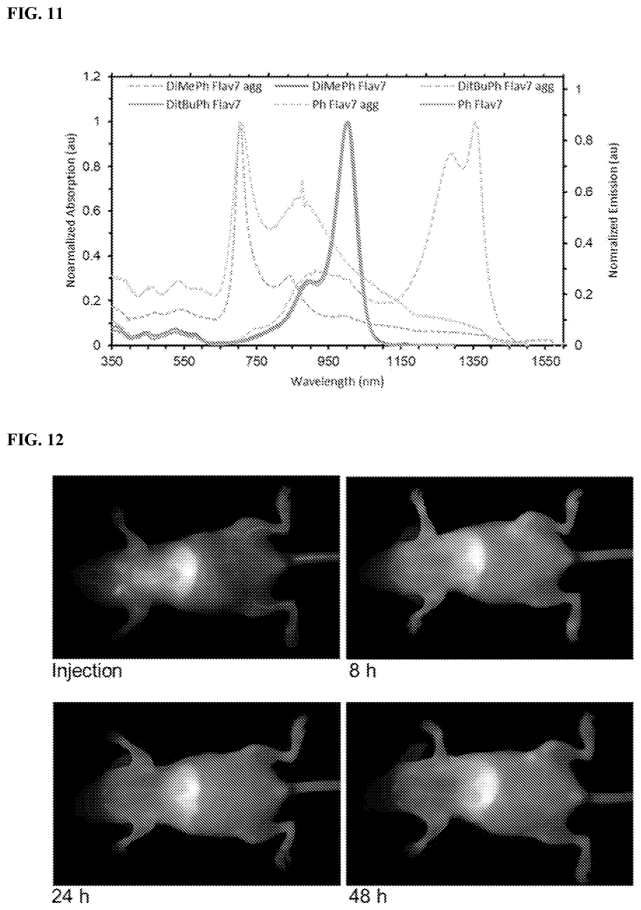

which exhibit the aggregation an UV-Vis absorption behavior(s) described in FIG. 11, under the following conditions: 0.2 mM dye in 4:6 EtOH: 0.9% NaCl/D₂O.

In certain embodiments, the disclosure describes dyes of the structures below:

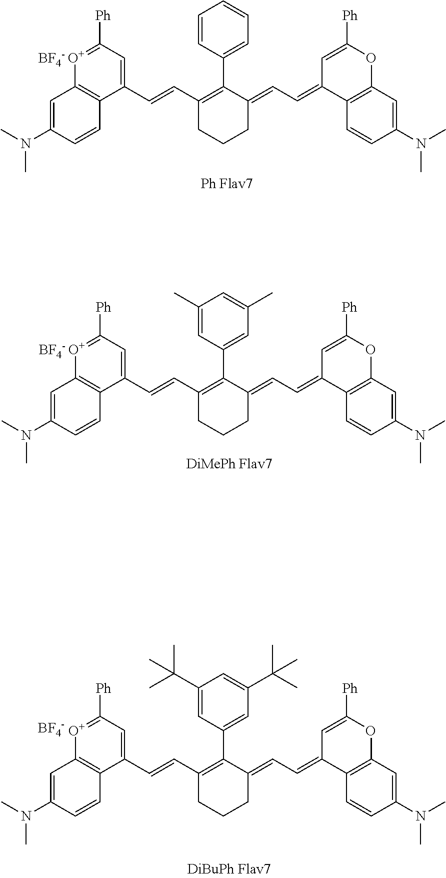

and the dyes, or micelle encapsulations thereof, exhibit the UV-Vis absorption behavior(s) described in FIG. 12.

In certain embodiments, the disclosure describes dyes of the structures below:

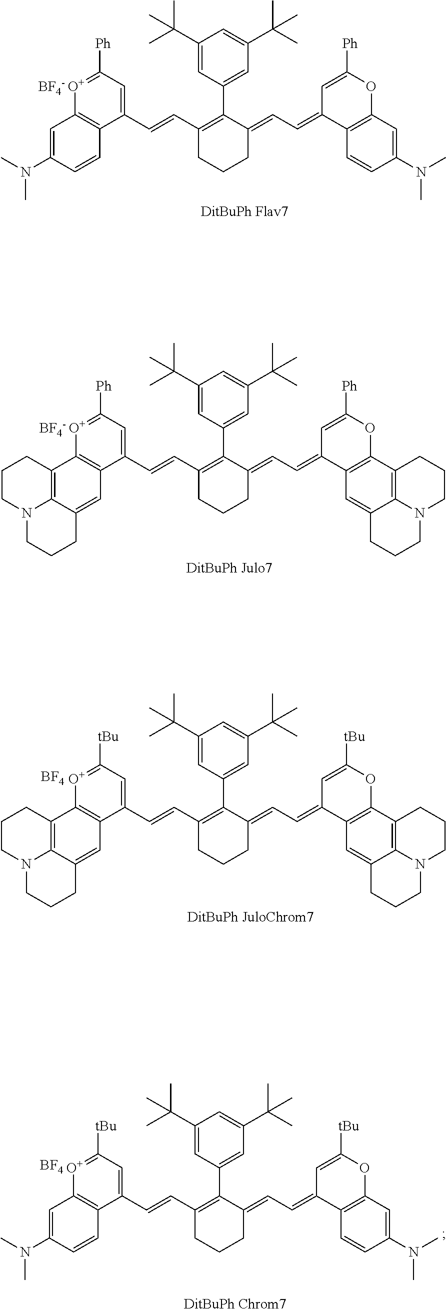

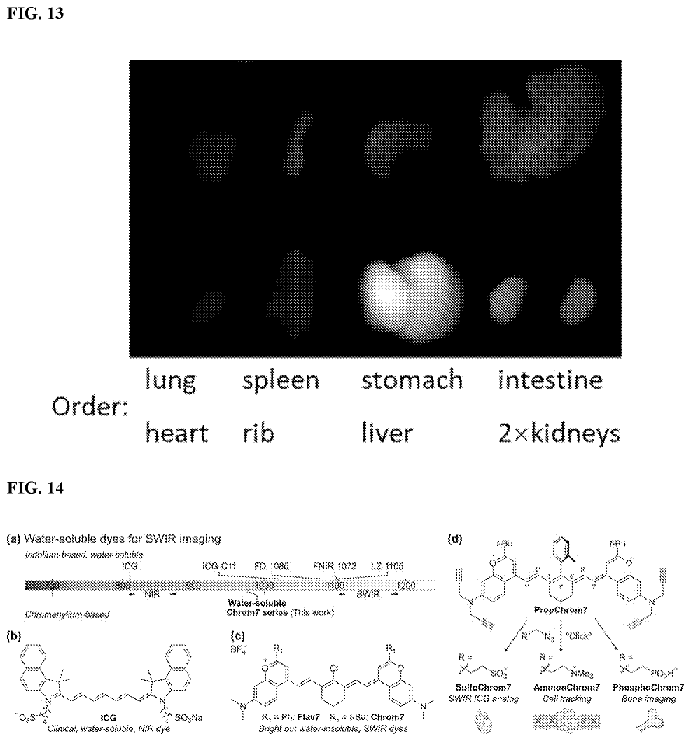

and micelle formulations thereof give the UV-Vis absorbance behavior shown in FIG. 13.

### Example 3: Photophysical Characterization

| Compound in solvent | λₘₐₓ/nm | εₘₐₓ/10⁴ M⁻¹cm⁻¹ | φ_{F} |
|---|---|---|---|
| Propargyl-Chrom7 in | 928 | 14 ± 1 | 1.18 ± 0.03 |
| MeOH | | | |
| Ammon-Chrom7 in | 938 | 16 ± 1 | 0.0118 ± 0.0004 |
| MeOH | | | |
| Ammon-Chrom7 in H₂O | 923 | 6.2 ± 0.2 | 0.0043 ± 0.0002 |
| Ammon-Chrom7 in FBS | 962 | 11 | 0.0058 ± 0.0003 |
| Zwit-Chrom7 in MeOH | 940 | 16.4 ± 0.4 | 0.0101 ± 0.0004 |
| Zwit-Chrom7 in FBS | 960 | 11.5 | 0.0032 ± 0.0003 |
| Sulfo-Chrom7 in MeOH | 938 | 16.5 ± 0.2 | 0.0106 ± 0.0002 |
| Sulfo-Chrom7 in FBS | 963 | 9.5 | 0.0056 ± 0.0003 |

### Example 4: Imaging in-Mouse Using Sulfo-Chrom7

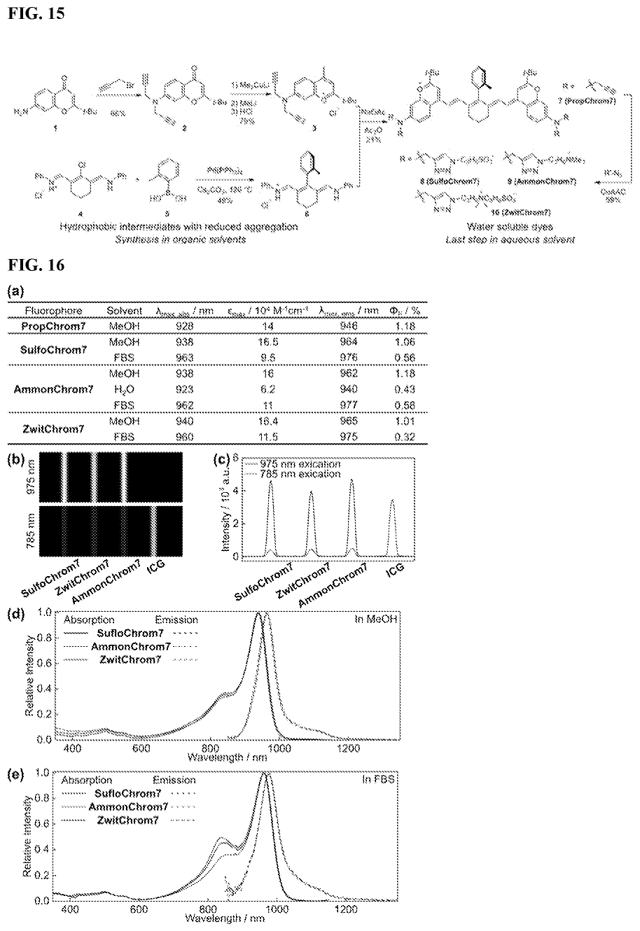

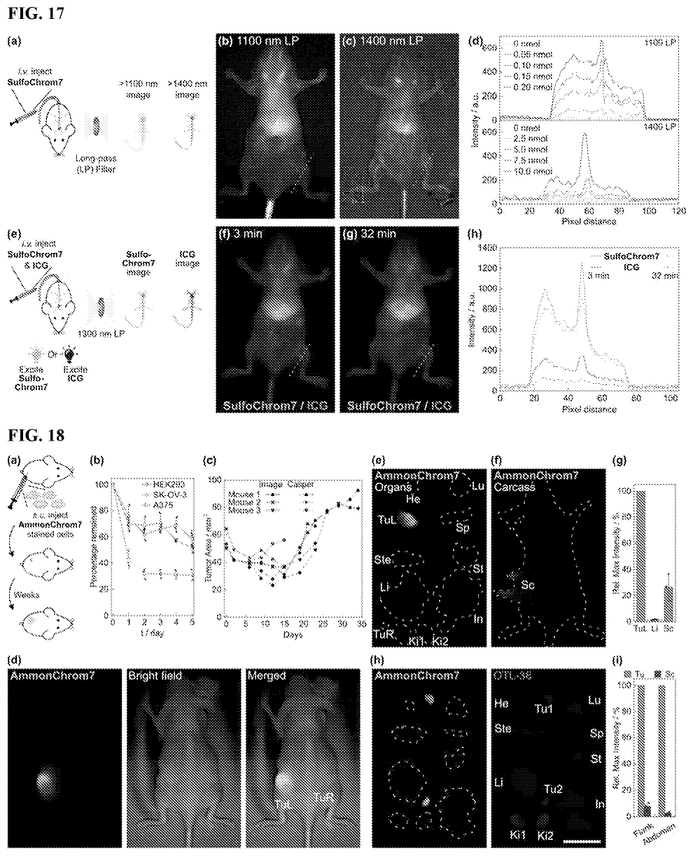

The results of imaging studies conducted in mice using Sulfo-Chrom7 are shown in FIGS. 14 and 15. FIG. 14 displays full-body images of a mouse at intervals over the course of 48 h starting at injection. FIG. 15 shows the distribution of the dye in the spleen, stomach, intestine, rib, liver, and both kidneys.

Experimental results, and exemplary synthetic schemes relating to the dyes of the present disclosure may be found in, e.g., US Patent Application Publication Nos. US2020/0140404 and US2021/0363124, the contents of which hare hereby fully incorporated by reference herein.

### Example 4: Imaging in-Mouse Using Compounds Disclosed Herein

### Summary

In vivo imaging using shortwave infrared light (SWIR, 1000-2000 nm) benefits from deeper penetration depths, decreased background autofluorescence, and high resolution. However, the development of biocompatible contrast agents for these low energy wavelengths has significant challenges. While there have been significant advances in SWIR chromophore scaffolds over the past 5 years, a major barrier for widespread utility of SWIR small molecule fluorophores is their hydrophobicity and tendency to form non-emissive aggregates. Here, we report a platform for generating a panel of soluble and functional dyes for SWIR imaging by late-stage functionalization of a fluorophore intermediate via click chemistry. The resulting fluorophores with sulfonate, ammonium or zwitterion functionalities are all water soluble with bright SWIR fluorescence in serum, allowing for fast imaging in mice. Specifically, the sulfonate-carrying derivative enables clear video-rate imaging of vasculature with as little as 0.05 nmol injected dye, and the ammonium-modified dye shows strong retention in cells that enables tracking of xenograft tumor growth. We further showcase the versatility of this design by incorporating phosphonate functionalities for imaging of bone in awake and moving mice. This modular design of functional SWIR fluorophores in water provides insights for facile derivatization of existing fluorophores to introduce solubility and bioactivity towards bioimaging applications.

Optical imaging utilizing shortwave-infrared light (SWIR, 1000-2000 nm; also referred to as NIR-II) is a rapidly growing research area. These long-wavelength photons benefit from reduced scattering, tissue absorption and autofluorescence compared to visible (400-700 nm) and near-infrared (NIR, 700-1000 nm) light. These qualities render the SWIR region well-suited to assist research in small model animals and expand the scope of optical clinical diagnostics. Indeed, in 2020, 11 years after the seminal report of SWIR imaging in mice, a study in humans concluded that SWIR detection was superior to NIR detection in image-guided surgery with higher tumor detection sensitivity and increased signal to noise ratio. The clinical trial employed indocyanine green (ICG, FIG. 14B), an FDA approved NIR fluorophore with a small percentage of emission in the SWIR region, as a contrast agent for their studies. If bright, SWIR analogs of ICG were available, the benefits of SWIR detection would be more pronounced for these surgical guidance procedures in terms of sensitivity and accuracy. Additionally, water soluble SWIR fluorophores will expand the scope of experiments that can be performed in model organisms.

ICG is a heptamethine dye with benzo[e]indolium heterocycles (FIG. 14B).

Polymethine dyes have significant advantages as optical contrast agents including small size, biocompatibility, and excellent absorption properties (narrow absorption bands with high absorbance coefficients (ε)). Consequently, polymethine dyes have seen considerable success as water soluble probes and fluorophores in the visible and NIR regions. Over the past five years, numerous SWIR-emissive polymethine dyes have been prepared using two red-shifting strategies: polymethine chain extension or heterocycle modification. While each of these approaches have been successful at producing fluorophores with excellent photophysical properties for the SWIR region in organic solvent, there are significant challenges in solubilizing these large, planar, aggregation-prone fluorophores in water. Polymethine chain extension is the most classic method to red-shift this class of fluorophores, but as the chain lengths are increased, the delocalization of the π-bonds across the polymethine chain can become compromised, leading to a molecule with poly-ene character that has unfavorable photophysical properties. This phenomenon is termed ground state desymmetrization and leads to broadened absorption bands with decreased absorbance coefficients and lowered quantum yields of fluorescence. Ground state desymmetrization is enhanced in polar aqueous media, rendering imaging of long chain polymethine dyes in physiological conditions more challenging. Heterocycle modification allows for SWIR fluorophores with pentamethine or heptamethine chains, decreasing contributions from ground state desymmetrization; however, these heterocycles are often more hydrophobic than the classic indolium heterocycles and the approaches commonly used to solubilize polymethine dyes in water are not successful on these more customized heterocycles. In fact, the small number of water soluble polymethine SWIR fluorophores to date all include indolium-derived heterocycles with polymethine chain extension, and varying amounts of ground state desymmetrization are observed in water (FIG. 14A).

Here we report a modular approach to water soluble SWIR-emissive chromenylium heptamethine dyes. The chromenylium heterocycle scaffold is a bright, red-shifted heterocycle for polymethine fluorophores (e.g. Flav7 and Chrom7, FIG. 14C). Chromenylium polymethines encapsulated in micelles have enabled SWIR imaging at record frame rates, with multiple channels, and using responsive FRET probes. To render the chromenylium heptamethine dyes water soluble, we determined two critical modifications are necessary: steric bulk on the polymethine linker to block π-π stacking and addition of multiple charged functionalities to impart sufficient water solubility. We prepare the clickable hydrophobic fluorophore PropChrom7, (FIG. 14D) which contains an ortho-methyl substituted phenyl group at the 4′-position to prevent aggregation and four propargyl groups for copper-catalyzed azide-alkyne cycloaddition (CuAAC) with charged azides to introduce hydrophilicity and/or functionality into the final product. PropChrom7 is a versatile intermediate for the preparation of a range of SWIR fluorophores with different functional groups and charge states. Using this approach, we obtained a panel of water soluble Chrom7 derivatives that carry sulfonates (SulfoChrom7), ammoniums (AmmonChrom7) and zwitterions (ZwitChrom7) with varying localization properties (FIG. 1d). These dyes all show monomeric dispersion in serum at dilute conditions with bright SWIR fluorescence. Notably, negatively-charged SulfoChrom7 shows slow absorption by tissue which allows for imaging of superficial veins with low detection limits; quintuply positively-charged AmmonChrom7 shows efficient uptake and prolonged retention in cells, enabling tracking of xenograft tumor growth in mouse models. We further showcased this late-stage functionalization design by incorporation of tetravalent phosphonates as both hydrophilic groups and calcium binding sites to achieve high-resolution bone imaging in both anesthetized and moving mice. We expect our platform will expand to provide water soluble SWIR fluorophores with varying photophysical properties and more bioactivity, as well as inspire more SWIR fluorophores packaged as modular building blocks to furnish complex animal imaging tools.

### Results and Discussion

### Design and Synthesis of Water Soluble SWIR Heptamethine Dyes.

When considering how to prepare water soluble derivatives of chromenylium SWIR dyes, we aimed to design a synthesis that would minimize water soluble intermediates and maximize diversity of the final fluorophores. We focused on first installing aggregation-minimizing functionalities on a hydrophobic scaffold that could then be transformed into a water soluble fluorophore through a click reaction with a charged functionality. To disfavor aggregation, we base our fluorophore on Chrom7 as its bulky di-tert-butyl substitution acts as steric hindrance that disfavors aggregation compared to planar Flav7, as evidenced by their micelle formulations. However, the two tert-butyl groups were not effective enough at reducing aggregation and an ortho-methylphenyl group was installed at the 4′ position of the polymethine chain. This substitution is analogous to the pendent aryl ring in Tokyo Green. Due to its steric demands, the 4′-substitution is perpendicular to the dye plane, which positions its protruding methyl group close to the polymethine bridge, thus preventing another dye molecule from effective π-π stacking necessary for aggregation to occur. These aggregation-minimizing features were consolidated into a tetra-propargylated chromenylium fluorophore, deemed PropChrom7. To promote the aqueous solvation of the fluorophores, we introduced hydrophilicity at the very last step, where we performed CuAAC between PropChrom7 with selected hydrophilic azides. We expect the four equivalents of hydrophiles to render the heptamethine dyes water soluble. Initially, we selected organic azides carrying sulfonate, ammonium, or zwitterion functionality.

PropChrom7 as a central building block enables the facile synthesis of the water soluble dyes in this work. The synthesis of PropChrom7 is carried out in organic solvents similar to previously reported chromenylium dyes. It is only in the last step converting PropChrom7 to the final water soluble fluorophore where aqueous solvent and HPLC separation were necessary (FIG. 15). Specifically, we obtained the chromone 1 from the microwave-assisted pyrone synthesis, utilizing allyl protection groups on the aniline that was compatible with high temperatures and pressures encountered in microwave synthesis. Following allyl group deprotection with Pd(PPh₃)₄, chromone 1 was obtained and the propargyl substitutions were installed by treatment with propargyl bromide to yield 2. The standard conditions for conversion of chromones to chromenylium dyes using MeMgBr or MeLi both gave poor yields due to reactivity with the propargyl groups. We found pre-treatment with Me₂CuLi provided an in situ protection of the terminal alkynes, allowing isolation of 3 in 79% yield. In parallel to the synthesis of propargylated chromenylium 3, we prepared polymethine linker 6 which contains the aggregation-blocking methyl-phenyl group. Linker 6 was constructed from Suzuki-Miyaura cross-coupling between commercially available compounds 4 and 5 at 120° C., which is harsher than the commonly-used condition for this type of conversion to compensate for the increased steric demands. PropChrom7 (compound 7) was prepared from the condensation of 6 and 3 in 21% yield. This central intermediate then underwent CuAAC with hydrophilic organic azides under a commonly-used condition for bioconjugation with THTPA as the ligand, but in a 1:2 mixture of water and methanol, to accommodate the solubility of both the hydrophobic dye and hydrophilic azide. This procedure resulted in 8 (SulfoChrom7), 9 (AmmonChrom7) and 10 (ZwitChrom7) all with ca. 59% yield.

### In Vitro Characterization of Water Soluble SWIR Fluorophores.

We first tested the photophysical behaviors of the dyes in different conditions (FIGS. 16A & 24A). All three hydrophilic dyes (Sulfo-, Ammon- and ZwitChrom7) and hydrophobic dye PropChrom7 show monomeric dispersion in MeOH and can be compared directly (FIGS. 16 and 20-23). By converting the propargyl groups on PropChrom7 to the triazolemethyl groups, the fluorophore exhibits a 10 nm red-shift in absorption and a 16 nm red-shift in emission maxima, corresponding to the increased electron donation from the triazoles (FIGS. 16A, 16D, and 20-23). All the hydrophilic dyes exhibit similar properties in absorption maximum and extinction coefficient, as well as fluorescent quantum yield (FIGS. 16A, 16D, and 16E), suggesting that the functionality appended to PropChrom7 can be varied without compromising the photophysical properties.

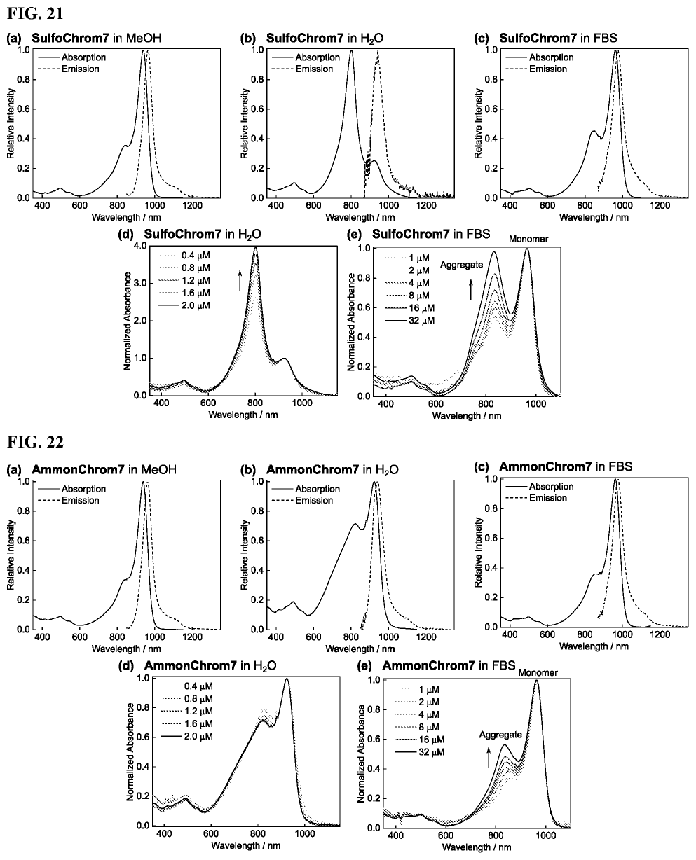

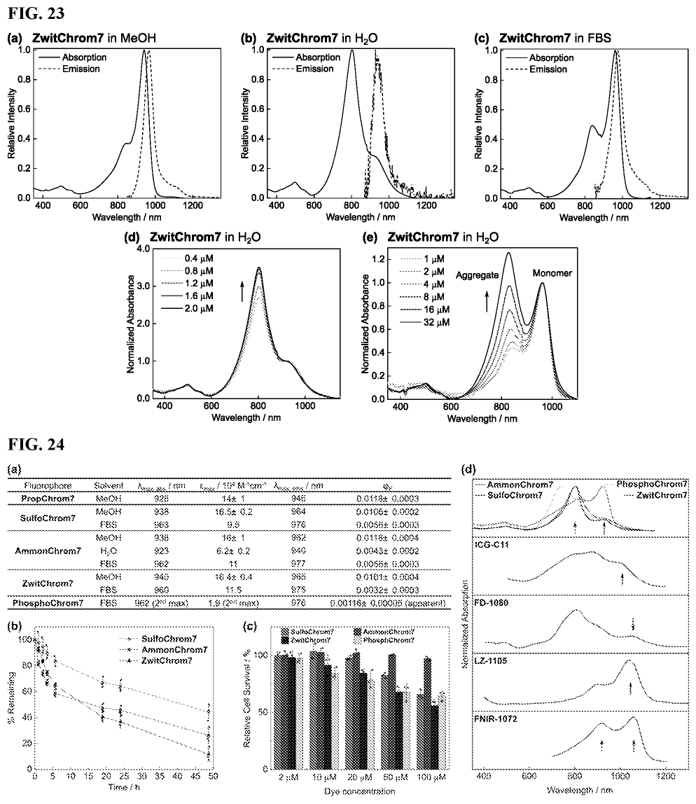

Owing to the structural homology of 8-10, we are able to compare the water-solubilizing ability of sulfonate, ammonium, and zwitterion functionalities. As shown in the absorption spectrum in ILO, ammonium salt possesses the strongest ability to solubilize the chromenylium fluorophore as evidenced by the dominant monomeric absorption profile of AmmonChrom7 at concentrations as high as 2 μM (FIG. S3b,d). Next best is the sulfonate group in SulfoChrom7 which shows a discernible monomer absorption (FIGS. 21B & 21D), whereas zwitterionic moiety in ZwitChrom7 is primarily aggregated in water (FIGS. 23B & 23D). While the dyes do display some aggregation in water, gratifyingly, there is minimal evidence of ground-state desymmetrization in H₂O, suggesting an advantage of heptamethine SWIR dyes (FIG. S5d).

Next, we evaluated the fluorophores in more biologically relevant fetal bovine serum (FBS) and found that all three fluorophores displayed a well-defined monomeric absorption and emission characteristic of polymethine dyes (FIG. 16E). AmmonChrom7 aggregates the least in FBS with monomeric absorption observed up to 32 μM, while SulfoChrom7 and ZwitChrom7 have dominant monomeric absorption up to 8 μM. Additionally, in FBS the absorbance of the three dyes were red-shifted by ca. 40 nm (FIG. 16E). These observations suggest that all three fluorophores are interacting with serum proteins, which is a behavior similar to that seen for ICG. The quantum yield values for AmmonChrom7 and SulfoChrom7 are above 0.5% in FBS, a notable metric for SWIR dyes in aqueous media. The ZwitChrom7 is slightly lower at 0.32% in FBS. Comparative capillary images in FBS between AmmonChrom7, SulfoChrom7, ZwitChrom7, and ICG with 785 nm or 975 nm excitation suggest all three SWIR dyes are comparable or superior to ICG for SWIR imaging (FIGS. 16B & 16C).

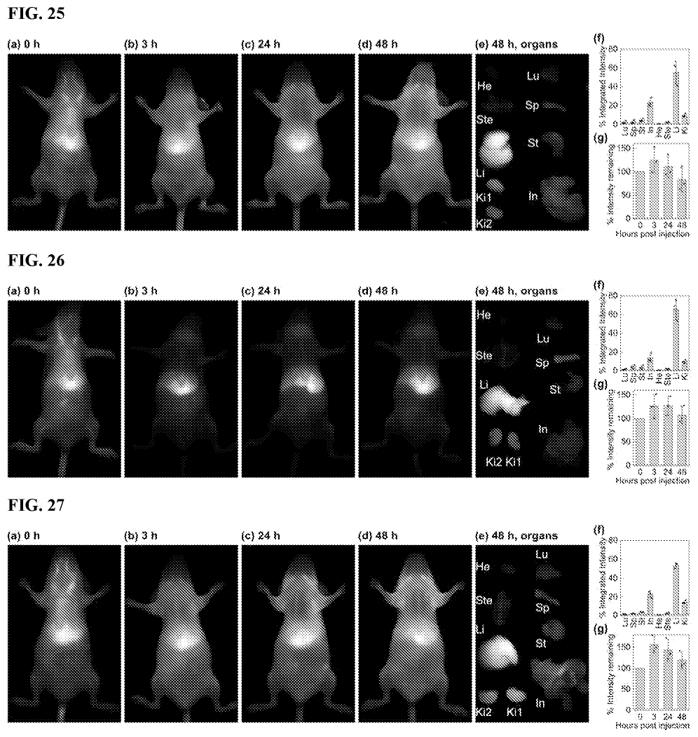

We further tested the dyes for their biocompatibility. The hydrophilic dyes display reasonable stability, with around ½ of SulfoChrom7, ¼ of AmmonChrom7 and 1/10 of ZwitChrom7 left over 2 days at 37° C. in FBS (FIG. S5b), which is in the same range as the degradation of ICG. The major degradation pathway of these dyes is attributed to oxidation of the fluorophore as determined by LC/MS. Inhibition of proliferation in HEK293 cells is minimal for AmmonChrom7 even with dye concentrations as high as 100 μM over 18 h, and the growth inhibition of SulfoChrom7 and AmmonChrom7 are also mild at 20 μM (<15% inhibition, FIG. 24C). As such, we deem these dyes suitable for use in animals at or below ca. 10 μM (20 nmol injection for 5-week or older mice) concentrations.

### Biodistribution of Water Soluble SWIR Fluorophores.

After in vitro characterization, we carried out in vivo imaging of the three hydrophilic dyes in mice to compare the differences in biodistribution. Towards this end, we injected 20 nmol of each dye into the tail vein, and observed a strong fluorescence signal for each dye which allowed imaging at 100 frames per second (fps, 0.5-2 ms exposure time, FIGS. 25-27) through an 1100 nm long-pass (LP) filter. All three dyes initially localized primarily in the liver and vasculature. After 3 h, almost all AmmonChrom7 accumulated in liver (FIGS. 26A-26D), whereas SulfoChrom7 and ZwitChrom7 showed diffuse signal through the mouse body, but still with substantial liver accumulation (FIGS. 25A-25D and 27A-27D). The distribution and brightness of all three dyes remain relatively unchanged over two days (FIGS. 25G, 26G, and 27G), indicating that they are not actively metabolized or secreted. In fact, their lasting fluorescence signals were retained much beyond their half-life in aerated FBS, suggesting significantly reduced degradation in vivo. Dissection of the organs 48 h after injection reveals that all the dyes stained primarily the liver (FIGS. 25E, 25F, 26E, 27F, 28E, and 28F). While AmmonChrom7 displays the strongest accumulation in the liver as noted, all three dyes showed some staining in the kidney and intestine, suggesting slow clearance through these pathways. Compared with ICG, which has a blood t_{1/2} of 10 min followed by hepatobiliary clearance within 6 h, the lasting in vivo fluorescence of our dyes offers a valuable reference for the future development of SWIR contrasting agents. The slower pharmacokinetics may enable flexible introduction time before clinical imaging. The long circulation time of these fluorophores also makes them ideal for long-term tracking of biological events in living mice for research purposes.

Systemic Imaging with SulfoChrom7.

The anionic SulfoChrom7 is the most similar of the fluorophores to ICG, the current benchmark for untargeted optical in vivo imaging, and appeared the brightest of the fluorophores in capillary imaging experiments. For these reasons, we performed a more extensive set of in vivo imaging experiments with SulfoChrom7 to determine its limit of detection and performance in comparison to ICG. Current clinics usually apply 2.5 mg (ca. 0.05 μmol/kg) ICG for detection of lymph nodes, tumors and vital structures under routine NIR imaging,⁴¹ whereas for SWIR imaging a much larger dose of ICG is required to compensate for the small fraction of the SWIR emission from ICG (0.3 or 0.6 μmol/kg in mouse, pig or human) but still with >100 ms exposure time. Considering that SulfoChrom7 is a bright fluorophore with majority of emission in the SWIR, we anticipate a very small dose of SulfoChrom7 is necessary for SWIR imaging. This represents an advance over ICG, since it is beneficial to introduce as little contrast agent as possible to minimize unnatural interactions and toxicity. To determine the relevant concentrations necessary for SWIR imaging with SulfoChrom7, we performed sequential tail vein injections of 0.05 nmol (ca. 2.5 nmol/kg) of SulfoChrom7. We set our desired imaging parameters at 33 fps (real time imaging) first with an 1100 nm LP filter (FIG. 16A). As soon as the first dose of 0.05 nmol dye was introduced, the liver lit up together with discernible saphenous and medial marginal veins, facial veins and abdominal wall veins (FIGS. 16B & 16D). As more dye was injected, the signal increased in a dose-dependent manner, giving rise to higher signal to noise ratio (FIGS. 16D & 28A-28F). This is, to the best of our knowledge, the smallest amount of dye directly administered without formulation to enable high quality video-frame rate non-invasive imaging.

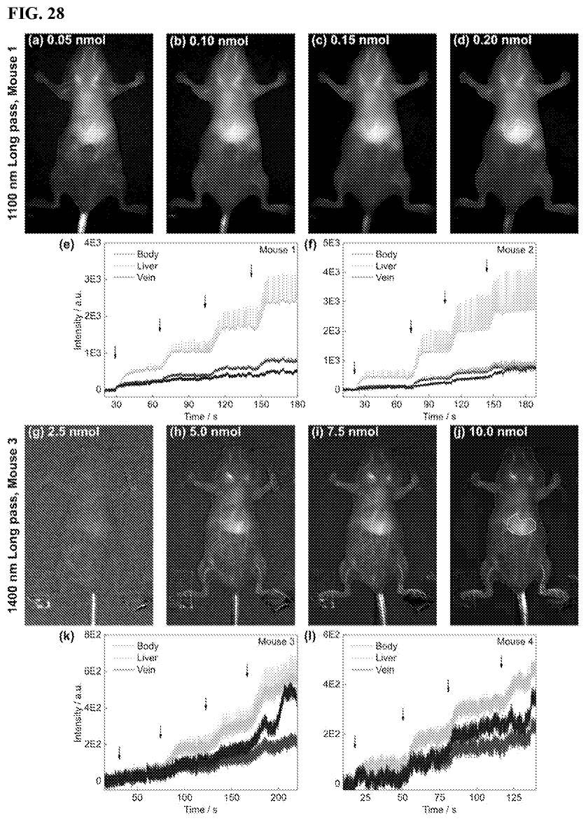

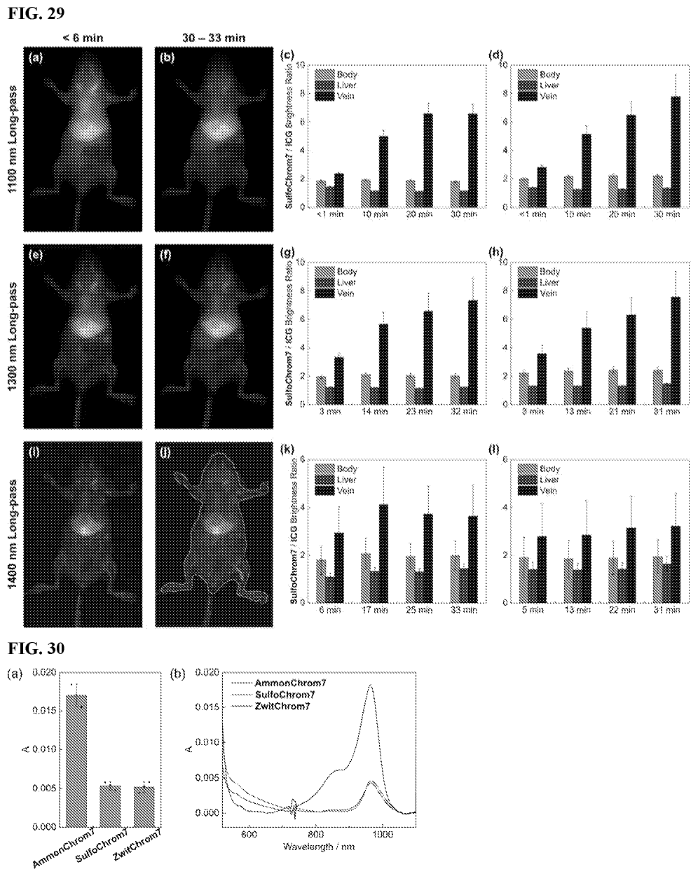

We then performed a similar set of experiments with more restrictive imaging metrics of 33 fps and a 1400 LP filter. Water molecules have considerable absorption of light beyond 1400 nm, which reduces signal from deeper tissue and at the same time enhances the resolution by attenuating scattered light (FIG. 16A). For these experiments, we increased the amount of dye injected to 4×2.5 nmol to compensate for the reduced integrated signal after 1400 nm (FIG. S2c) as well as the signal loss from water absorption. After 5.0 nmol dye was introduced, the fluorescence image clearly delineated a map of the vein system of the mouse, whereas the interference from liver accumulation was reduced due to the more significant depth of the liver (FIGS. 16C & 16D). As the amount of dye was increased, fluorescence signals increased with a more distinct vein map and reduced noise level (FIGS. 16D & 28G-28L). With 10 nmol SulfoChrom7, we were able to obtain a detailed image of superficial vasculature with minimal noise level (FIG. 28J). On the other hand, while ICG also lit up the vein system, the contrast was much weaker than SulfoChrom7 under the same settings because of the tiny emission tail at 1100 nm or longer wavelengths for SWIR imaging. Moreover, the observation window is much shorter, which we further assessed in the next step where we performed a direct comparison of SulfoChrom7 and ICG for vasculature imaging.

We are able to directly compare ICG and SulfoChrom7 as SWIR contrast agents in the same mouse because they can be selectively excited at different wavelengths (FIG. 16E, 785 nm for ICG, 975 nm for SulfoChrom7). For these experiments, we used a 1300 LP filter to compromise between resolution and signal intensity. As shown in the fluorescence image, immediately after injection, both dyes stained the vasculature with much stronger signal in the SulfoChrom7 channel owing to its red-shifted emission (FIGS. 16F & 16H). Within 30 min, most ICG accumulated in the liver with secretion into the intestine, leaving negligible signal in the mouse body, whereas SulfoChrom7 still reflected the superficial vasculature map while also displaying liver accumulation (FIGS. 16G & 16H). SulfoChrom7 displayed an increasing brightness ratio over ICG in veins over time (up to 7-8 fold, FIGS. 29E-29H). A similar trend is also displayed when imaging with 1100 or 1400 nm LP filter, where signals from SulfoChrom7 are retained in vasculature during the 33 min time frame while ICG clears into the liver and intestines (FIG. S10a-d,i-l). Taken together, SulfoChrom7 benefits from its higher SWIR brightness and longer blood circulation time than ICG, suggesting itself as an excellent SWIR fluorophore to visualize the superficial vein system over long periods of time with high sensitivity and adjustable resolution in different imaging setups.

Tracking of Tumor Growth with AmmonChrom7

Next, we explored the utility of the highly cationic AmmonChrom7 fluorophore. AmmonChrom7 features a permanent z=+5 and m/z of 269.6. This high charge density is close to those of some cell-penetrating peptides (e.g. nonaarginine m/z=159 at z=8, TAT peptide m/z=233 at z=7), suggesting its effective internalization into cells. Highly-cationic dye molecules have also shown effective cellular uptake and find utility in cell tracking applications, whereas many positively-charged nanoparticles has been developed for such purpose. We thus anticipated that AmmonChrom7 could be similarly internalized into cells for long-term visualization (FIG. 20A). To test this hypothesis, we incubated HEK293 cells with 50 μM of AmmonChrom7, SulfoChrom7, and ZwitChrom7 and analyzed the absorbance of cell lysates over time. We found significant absorbance for cells treated with AmmonChrom7, whereas the signal for SulfoChrom7 and ZwitChrom7 is minimal (FIGS. 30A & 30B). The cellular labeling of AmmonChrom7 dropped significantly on the first day after staining, possibly due to exocytosis from saturated cytosol, and then decreased fairly slowly to retain a signal around 50-70% over five days (FIG. 18B). Similar behavior was also observed in A375 cells, a human melanoma cell line, and SK-OV-3 cells, a human ovarian cancer cell line (FIG. 18B). These data, in combination with the low cytotoxicity (FIG. 24C), slow clearance (FIG. 26), non-aggregation behavior and high SWIR brightness in aqueous solutions (FIGS. 15A, 15D, 15E, and 22) make AmmonChrom7 a great candidate for cell tracking experiments.

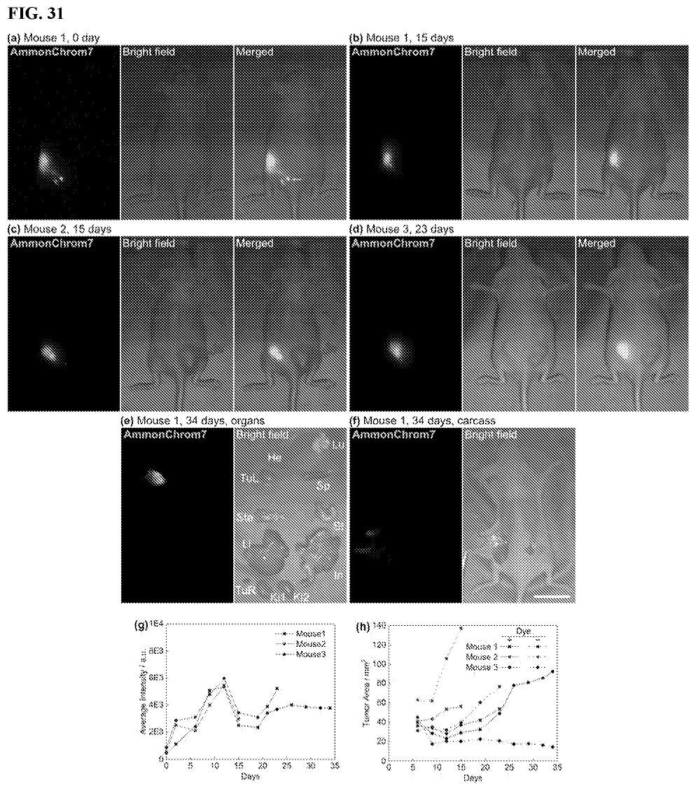

To showcase the excellent retention of AmmonChrom7 in cell tracking experiments, we monitored the in vivo growth of xenograft tumors in mice. We first stained A375 cells with 50 μM AmmonChrom7 for 18 h prior to their s.c. injection into the left flank of mice to track their growth as xenograft tumors (FIG. 18A), with non-stained cells injected to the right flank as control. The stained cells initially showed spread out and smeared fluorescent distribution upon injection (FIGS. 18C & 31A), and then became coagulated within the first two weeks, corresponding to the assimilation of Matrigel that initially supported the cells (FIGS. 18C & 31B). In the meantime, the brightness of the stained tumor increased corresponding to the increased dye density, allowing for image capturing with as short as 1 ms exposure time (FIG. 31G). As the cells started to divide, the size of the fluorescence area gradually increased, which correlates well with the caliper measurement (FIG. 18C). While the overall mean fluorescence decreased upon tumor growth due to the dilution of the dye (FIG. 31G), some regions of the tumor showed lower signals (FIGS. 18D, 31C, and 31D), possibly as indicators of actively-growing sites versus more senescent locations of the tumor. Notably, the fluorescence signal was well-contained at tumor sites as seen in the bright-field image (FIGS. 18D & 31B-31D). We sacrificed the mice at varying dates depending on the size of the two tumors to obtain ex vivo images. As expected, the three stained tumors all showed bright fluorescence under SWIR camera, whereas we observed much lower fluorescence in the surrounding tissue, negligible intensity in the liver, and essentially no fluorescence in the control tumors and the rest of the body (FIGS. 18E-18G). These results further confirm that AmmonChrom7 was contained at the tumor site during their growth with negligible leakage. Additionally, the 3/3 take rate and comparable growing speed (FIG. 31H) suggest an insignificant long-term inhibition effect of AmmonChrom7 to the A375 tumor growth.

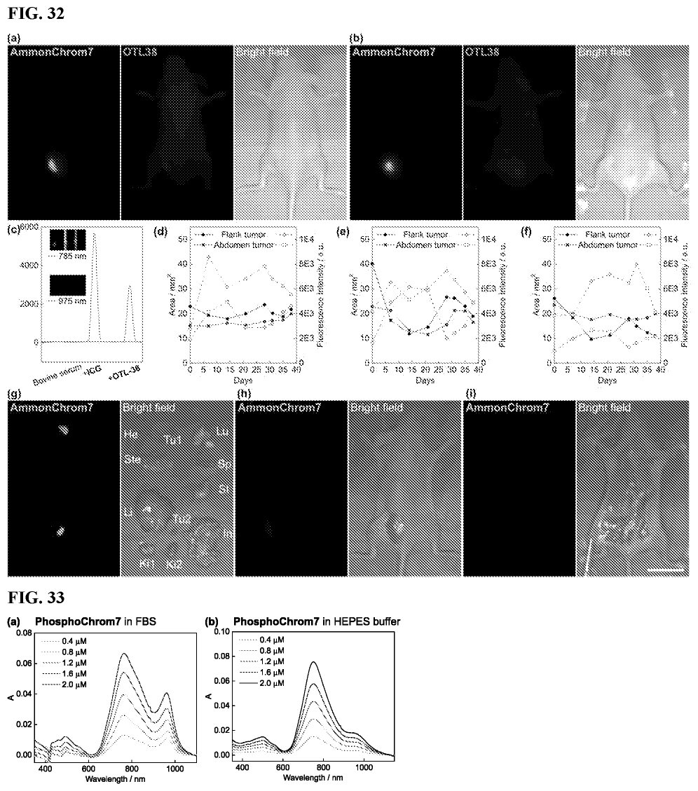

Next, we performed a similar experiment with the more challenging, slow growing, xenograft model SK-OV-3. We treated SK-OV-3 cells with 50 μM AmmonChrom7 prior to s.c. injection in the flank and abdomen of three mice. Again, the fluorescence signal showed clear overlap with the tumor location from bright-field images (FIGS. 32A & 32B). The size of the tumors remained largely unchanged since week 1, and the fluorescence intensity fluctuated accordingly, rather than decreasing (FIGS. 32D-32F), suggesting that the dye was retained at the tumor site with little diffusion or degradation over the 38-day period. To our knowledge, this sets the record for the longest time period for a SWIR dye to be monitored in vivo. To confirm that the fluorescence originated from the tumor, we injected OTL-38, a recently FDA-approved NIR dye that labels the overexpressed folate receptor on SK-OV-3 cells. OTL-38 contains a NIR heptamethine analogous to ICG as the fluorophore core and thus we envisioned the tail emission in the SWIR could be utilized for imaging (FIG. 32C). As expected, clear colocalization was observed in in vivo images (FIGS. 32A & 32B) and ex vivo images (FIG. 18H), supporting that the dye continuously labels the SK-OV-3 tumor throughout the period of the experiment. While much non-specific accumulation of OTL-38 was observed in other organs, AmmonChrom7 showed clear staining of the tumor with very little diffusion into the surrounding tissue (FIGS. 14H, 14I, and 32G-321), highlighting the cell tracking potential of AmmonChrom7. Taken together, these experiments demonstrate that by incorporating multiple positive charges onto our dye scaffold, we successfully engineered AmmonChrom7 as a cellular stain for the long-term tracking of tumor growth, offering the first small molecule option for in vivo cell tracking using SWIR imaging. The high brightness and minimal cell growth inhibition provide a valuable tool for studying cancer models, as well as potential use in stem cell and immune cell research.

Targeted Imaging with Water Soluble SWIR Fluorophores.

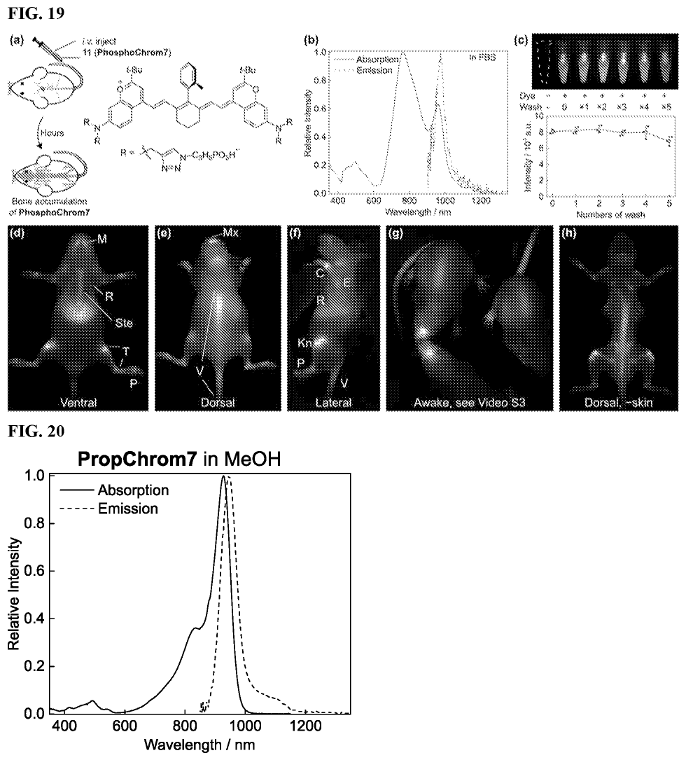

Initially, we focused on appending functionality to PropChrom7 that would induce water-solubility; however, a major advantage of PropChrom7 is the versatility of functionality that can be appended onto the SWIR chromophore. To showcase this, we prepared 11 (PhosphoChrom7) for bone-targeted imaging (FIG. 19A). Four phosphate groups were clicked onto PropChrom7 in 20% yield with the phosphate groups playing a dual role of hydrophilic and Ca²⁺-binding moieties. The unique binding affinity of phosphonates towards divalent metals has previously been harnessed in development of bone-targeting drugs and NIR bone imaging agents. We thus anticipated that PhosphoChrom7, with its four phosphonates, could be delivered without a carrier and facilitate skeleton imaging due to its affinity for bone.

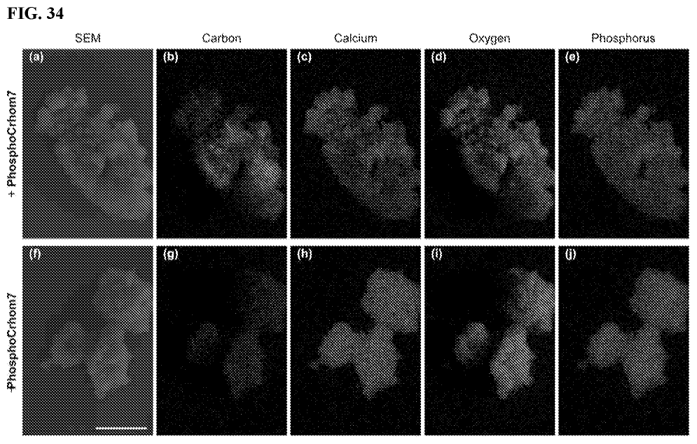

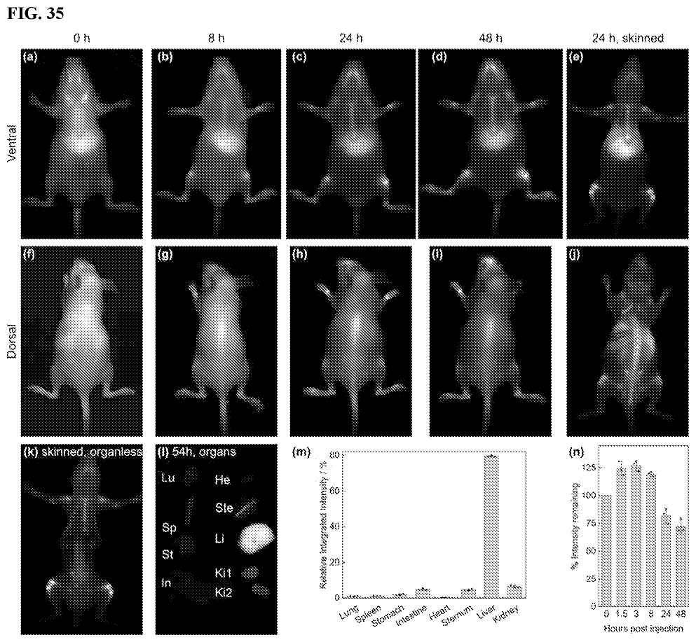

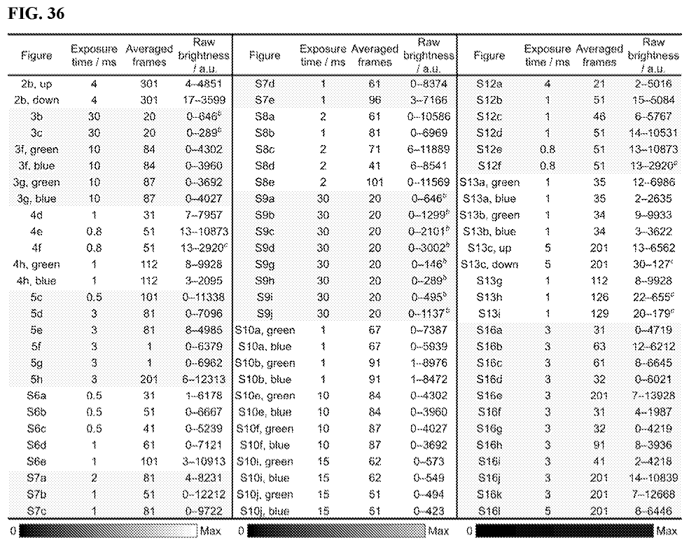

We first analyzed PhosphoChrom7 in in vitro assays. The solubility of this compound in FBS is lower than the Sulfo-, Ammon-, or ZwitChrom7 (FIG. 33). The lower solubility is consistent with trends seen for bisphosphonate drugs. The photophysical consequences of the decreased solubility is a broad aggregation peak at 760 nm. Interestingly, this aggregate peak is significantly blue-shifted compared to the other three water soluble dyes, suggesting a different mode of aggregation induced by the phosphonate moieties (FIGS. 19B and 33). Conversely, the absorption/emission wavelengths of the monomer peak are almost identical to the other three dyes (FIGS. 19B & 33A), providing further evidence that the functionality attached to PropChrom7 does not drastically affect the photophysical properties of monomeric fluorophore core.

We tested the binding affinity towards hydroxyapatite, the major mineral component of bone. Incubation with PhosphoChrom7 in bovine serum led to a bright suspension of the particles under SWIR camera, and most importantly, sequential washes with bovine serum only slightly decreases the overall brightness, indicating strong binding affinity of PhosphoChrom7 towards hydroxyapatite (FIG. 19C). To rule out the possibility of co-precipitation of the dye with the hydroxyapatite powder due to its limited solubility, we performed scanning electron microscopy on the particles. While we were unable to observe standalone particles without calcium and phosphorus after dye-treatment, we detected a significant increase of carbon element in energy dispersive X-ray spectrometry (EDS) mapping for the dye-treated particles compared to untreated ones (FIG. 34), suggesting that the dye was indeed adsorbed onto the calcium salt. The toxicity of PhosphoChrom7 is also mild at low concentrations (FIG. 34C), similar to Sulfo- and ZwitChrom7. Collectively, these properties of PhosphoChrom7 meet the criteria for bone-targeted SWIR imaging.

We then performed mouse imaging using this tetraphosphonate SWIR fluorophore. After i.v. injection, PhosphoChrom7 initially localized in the vasculature (FIGS. 35A & 35F), but gradually redistributed and began to feature bone structures at 8 h (FIGS. 35B & 35G), along with some accumulation in the liver. Aside from strong liver staining, the reduction of non-bone-localized signals gave rise to a contour of mouse skeleton after 24 h or 48 h (FIGS. 19D-19F, 35C, 35D, 35H, and 35I) with slightly faster clearance compared to previous hydrophilic dyes (FIG. 35N). While the dye may localize more onto bone regions with higher osteoblast or circulation activity, the SWIR image clearly outlines the mandible, sternum, tibia and phalange bones on the ventral view (FIG. 19D) and maxilla and vertebra on the dorsal side (FIG. 19E). The rib cage can be clearly visualized on the lateral view when the skin was gently lifted around the shoulder to reduce skin scattering (FIG. 19F). The high brightness of PhosphoChrom7 also enables bone imaging in awake and moving mice with comparable details (FIG. 20G). Most importantly, all these features were readily identifiable with high resolution in living mice without skin removal. Nonetheless, when the skin was removed from an euthanized mouse, more details of its bone structure were revealed (FIGS. 35E & 35J). Images of the dissected organs indicate that although bone was clearly stained by PhosphoChrom7, the liver is the major target of the dye similar to our other hydrophilic dyes (FIGS. 35L & 35M). We further removed the abdominal organs to eliminate their interference and were able to obtain a high-contrast image of the skeleton of the mouse (FIGS. 20H & 35K). Collectively, by facile introduction of calcium-binding phosphonates to our tetravalent functionalization platform, PhosphoChrom7 stands out as the first reported bone-targeting SWIR fluorophore. PhosphoChrom7 enables non-invasive optical imaging of bone at video rate speeds, providing a platform for studying osteology and bone-related diseases in model animals.

### Concluding Remarks

To close, we herein have reported a modular platform that uses PropChrom7 as a central intermediate with four conjugation handles to easily access a series of functionalized SWIR fluorophores via CuAAC. Through this platform, we obtained Sulfo-, Ammon-, Zwit- and PhosphoChrom7 as hydrophilic/water soluble SWIR dyes. All these dyes display minimal aggregation and ground state desymmetrization in serum. They exhibit bright SWIR fluorescence when i.v. injected in mice and can be imaged with video frame rates. Notably, these dyes readily dissolve in buffer as homogeneous solutions for convenient, direct administration, without concerns of batch variation, storage instability or potential in vivo breakdown which are frequently encountered for micelle formulations.

Besides the increased hydrophilicity imparted by the click reaction, this family of SWIR fluorophores offers versatile imaging tools. In particular, the anionic dye SulfoChrom7 stands out as a red-shifted analog of ICG with greatly enhanced SWIR brightness and longer circulation time. SulfoChrom7 facilitates imaging of mouse liver and vasculature with as little as 0.05 nmol, the smallest amount reported of contrast agents that enabled video rate imaging. The cationic dye AmmonChrom7 enables the monitoring of xenograft tumor growth over weeks with minimal signal loss and little diffusion into other tissues owing to its excellent brightness, biocompatibility, in vivo stability and cellular retention. Our tumor tracking experiments also set the record of in vivo detection time length for SWIR dyes. The cell tracking capability of this dye has the potential for use in monitoring cell activities in in vivo studies on tumorigenesis and immune cell migration. Lastly, PhosphoChrom7 exhibits strong binding to calcium minerals due to the four phosphonate groups on the molecule, furnishing the first bone-targeting fluorophore in the SWIR. Its brightness and reduced scattering of SWIR light eliminate the need for sacrificing, skinning or even anesthesia of the mice for high resolution bone imaging, enabling non-invasive video recording of the skeleton in awake, moving mice.

Our design also serves as a starting point for the modular design of water soluble, functional and formulation-free SWIR fluorophores for in vivo imaging. By separating the fluorophore synthesis and the introduction of water-solubility and/or bioactivity, this late-stage modification method allows for the facile combination of the fluorophore core and various functional groups tailored towards specific imaging requirements without worries about the functional group compatibilities and tedious aqueous purification during the synthesis of the fluorophore core. While our current tetravalent modification is expected to enhance targeting through avidity, it is also beneficial to explore other topology choices such as four water-solubilization groups and a single bioactive moiety. Our group is currently expanding the scope of both the packaged fluorophore core and other hydrophilic/bioactive functionalities to extend the platform for targeted and formulation-free SWIR imaging, with an overarching goal of providing user-friendly building blocks of SWIR detection reagents similar to the success of many commercially-available activated fluorophores in the visible region for easy bioconjugation. Finally, this fluorophore functionalization method on its two aniline groups also provides reference to future development of other aniline-containing molecules as modularized imaging agents and, more broadly, as other chemical biology tools.

### Procedures and Characterization Methods

### Dye Handling and Storage

All dyes were stored in pure solid form after purification in −20° C. freezer. Stock solutions of Sulfo-, Ammon- and ZwitChrom7 were prepared as 4 mM solutions in H₂O. Stock solutions of PhosphoChrom7 were prepared as 1 mM solution in H₂O containing 1 mg/mL of K₂CO₃. Leftovers of Sulfo-, Ammon- and ZwitChrom7 stock solutions were stored frozen at −20° C. for immediate use within a month.

### Photophysical Characterization

Absorption spectra were collected on a JASCO V-770 UV-visible/NIR spectrophotometer after blanking with the appropriate solvent. Photoluminescence spectra were obtained on a Horiba Instruments PTI QuantaMaster Series fluorometer. Fetal bovine serum (FBS) or bovine serum were purchased from Gibco. Quartz cuvettes (10 mm×10 mm) were used for absorption and photoluminescence measurements unless otherwise noted. All spectra were obtained at ambient temperature. Fluorescence quantum yield was measured with 830 nm excitation using Flav7 as a reference (Φ_{F}=0.61%).

### Stability Assay in FBS

Dyes were diluted to 1 μM in 1 mL of FBS containing 0.02% w/v NaN₃ and placed in disposable cuvettes. The cuvettes were sealed with parafilm and placed in 37° C. incubator until given time points. Absorption spectra were taken using the maximum absorption to represent the dye concentration. Each condition was performed in triplicate.

### Cell Culture

Cells were purchased from the American Type Culture Collection (ATCC). HEK293 cells were cultured in MEM medium supplemented with 10% FBS, 2 mM glutamine, 100 mM sodium pyruvate and 1% penicillin-streptomycin. A375 cells were cultured in DMEM medium supplemented with 10% FBS and 100 mM sodium pyruvate and 1% penicillin-streptomycin. SK-OV-3 cells were cultured in McCoy's 5A medium supplemented with 10% fetal bovine serum, 2 mM glutamine and 1% penicillin-streptomycin. All cells were cultured at 37° C. under 5% CO₂.

### MTT Toxicity Assay

HEK293 cells were split into three cultures as replicates. For each replicate, HEK293 cells were passed onto flat-bottomed 96-well plate to 40-50% confluency and left 6 h for adhesion. Different concentrations of compounds were added to the medium (1:40 dilution from aqueous stock solution) in quadruplicate and incubated for 18 h. After incubation, cells were treated with 100 μL of full media containing 0.5 mg/mL MTT for 2 h, followed by addition of 100 μL of aqueous solution containing 10% SDS and 1:1000 HCl. After 3-4 h incubation, cell viability was determined by measuring the absorbance at 490 nm using a plate reader.

### Uptake and Retention of AmmonChrom7 in Cells

HEK293, SK-OV-3 or A375 cells were split into three cultures as replicates. For each replicate, cells were passed onto 12-well or 24-well plates and grown until ca. 80% confluency. Each well was incubated with 50 μM of AmmonChrom7 for 6 h, and the cells were washed with PBS and incubated in MEM medium supplemented with 1% FBS. At given time points, the cell media were removed and cells were lysed with lysis buffer (150 μL, containing 1% Triton X-100, 0.1% w/v SDS and 0.1% w/v sodium deoxycholate) and diluted with bovine serum (300 μL). The cell lysate was collected and frozen at −20° C. until analysis. To quantify dye content, maximum absorption was used to represent the dye concentration. Scattering was estimated and subtracted with absorption at 1050-1100 nm, using the following equation for fitting:

$$A = log \frac{1}{1 - \frac{c}{λ}} + {A}_{0}$$

### Fluorescence Tracking of Growth of Tumor Xenografts

A375 or SK-OV-3 cells were grown to confluency and incubated with full media supplemented with 50 μM of AmmonChrom7 for 12 h. Cells were then incubated with full media for 6 h, followed by digestion with trypsin-EDTA. The cell pellet was further washed with PBS (×3). For A375 cells, stained or unstained cells were suspended in 1:1 PBS and Matrigel; 100 μL of cell suspension (3.3 million cells) were injected to each site. Tumor 2D-projected sizes A were monitored by caliper measurement of long diameter a and short diameter b of the tumors and estimated by the following equation:

$$S = \frac{1}{4} π {ab}^{2}$$

For SK-OV-3 cells, cells were suspended in 1:1 PBS and Matrigel; 100 μL of cell suspension (2.5 million cells) was injected to each site. Mice were fed on folate-deficient diet (Teklad) on day 21. On day 39, 10 nmol of OTL-38 in PBS was i.v. injected 4 h prior to imaging.

### Hydroxyapatite Binding Assay

Hydroxyapatite powder was purchased from Acros Organics. To a suspension of 10 mg of hydroxyapatite in 100 μL of bovine serum was added 50 μM PhosphoChrom7 and the mixture was placed on a revolver for 30 min for initial binding, followed by centrifugation to remove supernatant. For washing, the pellet was resuspended in 100 μL of bovine serum by vortexing and placed on a revolver for 30 min followed by centrifugation to remove supernatant. The pellet was finally resuspended in 100 μL of bovine serum for imaging under the SWIR camera. For quantification, each centrifuge tube containing the suspension was recorded at the center of the illumination/viewing field to calculate mean fluorescence intensity at the bottom area of the tube.

### SEM Imaging of Hydroxyapatite

Hydroxyapatite powder (40 μg) was suspended in 20 μL of 50 μM PhosphoChrom7 and placed on a revolver for 30 min for binding, followed by centrifugation to remove supernatant. This binding step was performed 3 times in total to increase the dye loading. The resulting brown solid was subsequently washed with MeOH for analysis.

Samples for SEM study were prepared by dropcasting the samples in a MeOH suspension onto a silicon wafer. SEM/EDS analysis was carried out on a ZEISS 1550VP Field Emission SEM-Oxford EDS-HKL EBSD system. During the measurement, the accelerating voltage was 15 kV and the working distance was kept at 6 mm.

### Animal Procedures

Animal experiments were conducted in conformity with guidelines from the University of California, Los Angeles with protocols approved by the Animal Research Committee (protocol number ARC-2018-047). Non-invasive whole mouse imaging was performed on athymic nude female mice (5-15 weeks old), purchased from Charles River Laboratories. Mice were anesthetized with inhaled 2-4% isoflurane. Tail vein injections were performed with a catheter assembled from a 29-gauge needle connected through plastic tubing to a syringe prefilled with isotonic saline solution. The bevel of the needle was then inserted into the tail vein and secured using tissue adhesive. The plastic tubing was then connected to a syringe (30-gauge needle) prefilled with the compound of interest. All solutions were filtered through a 0.22 μm syringe filter prior to i.v. injection.

### SWIR Imaging Apparatus

Imaging instrument was installed according to published procedure, using an Allied Vision Goldeye G-032 Cool TEC2 camera with illumination from LU0975DLU350-S30AN03 (35 W, 975 nm laser) and LU0785D250-U70AN (25 W, 785 nm laser) Lumics laser units to record images at 14-bit depth. Illumination was adjusted to 100 mW/cm² for 975 nm and 50 mW/cm² for 785 mm. Excitation was provided at 975 nm for Sulfo-, Ammon-, Zwit- and PhosphoChrom7, and at 785 nm for ICG. Fluorescence images were recorded with an 1100 nm long-pass filter unless otherwise noted. Dual-channel imaging (SulfoChrom7/ICG, or fluorescent imaging/bright field imaging) was performed with pulsed excitation that matches the detection window of the camera, or persistent ambient fluorescent lighting for bright field images to be subtracted as background in the fluorescence channel.

### Image Analysis

Images were processed using the Fiji distribution of ImageJ. All images were background subtracted to correct for non-linearities in the detector and/or excitation. Unless otherwise noted, all still images are produced from averaging of ≥20 frames, and displayed after adjusting brightness/contrast without pixel saturation in the mouse body. Videos were saved as raw avi and cropped, frame-rate adjusted and compressed with FFmpeg. For dual-channel imaging, emission of the SulfoChrom7 and AmmonChrom7 in 785 nm channel was estimated to be 9.0% intensity of their 975 nm image as derived from the capillary experiment, and subtracted from the 785 nm channel.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of formula I: 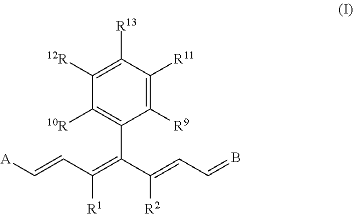
wherein:
R¹ and R² are each independently selected from H, alkyl, or halo; or R¹ and R² together complete a cycloalkenyl ring, a heterocyclyl ring, or a polycyclyl ring system;
R⁹ is H, alkyl, alkoxy, haloalkyl or halo;
R¹⁰ is H, alkyl, alkoxy, haloalkyl or halo;
R¹¹ and R¹² are each independently H, C₃-C₁₀ alkyl or cycloalkyl; and
R¹³ is H, alkynyl-alkyl, alkynyl, heteroaralkyl, heteroaryl, a group comprising an azide, heteroaralkyl, or a moiety that comprises a reactive group capable of undergoing bioconjugation, or a group comprising an acid, aldehyde, alkene, hydroxyl, amide, urea or sulfonamide; and
A and B are each independently selected from a bicyclic, tricyclic, or tetracyclic heteroaryl; wherein A and B are each independently substituted with one or more R^{14a} and/or R^{14b},
wherein each R^{14a} and/or R^{14b} is independently selected from H, alkoxy, acyl, heteroaryl, sulfonate, carbonate, cyano, ester, amide, halo, aryl, amino, alkylamino, C₁₋₆ alkyl, C₃₋₁₀ cycloalkyl, haloalkyl, aralkenyl, aralkynyl, hetaralkenyl, hetaralkynyl, and heterocyclyl;
or two adjacent R^{14a} and/or R^{14b} groups combine to form a carbocyclic or heterocyclic ring including the atoms to which they are attached.

2. The compound of claim 1, having the structure of formula Ia: 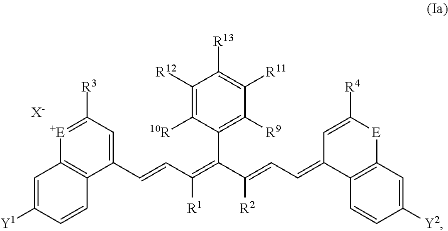
wherein:
E is O or S;
X is selected from halide and BF₄⁻ perchlorate, B(aryl)₄⁻, boron clusters, and TRISPHAT (tetrabutylammonium phosphorus(V) tris(tetrachlorocatecholate));
Y¹ and Y² are each independently H or Y¹ is —N(R⁵)(R⁶) and Y² is —N(R⁷)(R⁸);
R³ and R⁴ are each independently H, optionally substituted phenyl, optionally substituted heteroaryl, alkyl, or cycloalkyl;
each R⁵, R⁶, R⁷ and R⁸, when present, are each independently alkyl, alkynyl-alkyl, alkynyl, heteroaralkyl, heteroaryl, a group comprising an azide or a moiety that comprises a reactive group, or a group comprising an acid, aldehyde, alkene, hydroxyl, amide, urea or sulfonamide.

3-4. (canceled)

5. The compound of claim 1, having a structure of formula Ia: 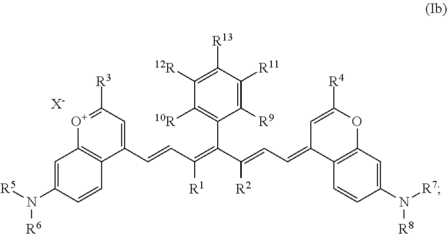
wherein X is selected from halide and BF₄⁻.

6. The compound of claim 1, wherein R¹ and R² together complete a cycloalkenyl ring.

7. The compound of claim 2, wherein R³ and R⁴ are phenyl; or wherein R³ and R⁴ are t-butyl.

8. (canceled)

9. The compound of claim 2, wherein Y¹ is —N(R⁵)(R⁶) and Y² is —N(R⁷)(R⁸).

10-11. (canceled)

12. The compound of claim 1, wherein the compound has a structure given by formula Ic: 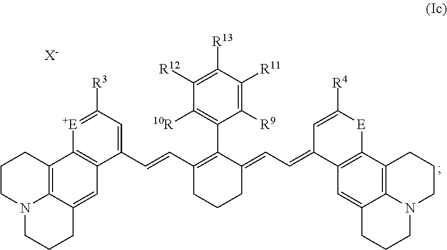
wherein X⁻ is selected from halide and BF₄⁻.

13-20. (canceled)

21. The compound of claim 12, wherein R¹¹ and R¹² are trifluoromethyl.

22-26. (canceled)

27. The compound of claim 1, having a structure of formula Id: 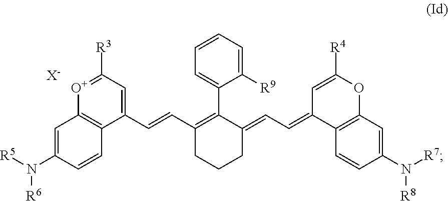
wherein X is selected from halide and BF₄⁻.

28-29. (canceled)

30. The compound of claim 27, having a structure of formula II:

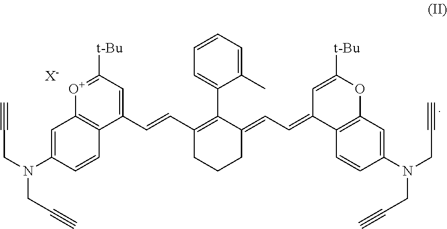

31-32. (canceled)

33. The compound of claim 1, having a structure of formula III: 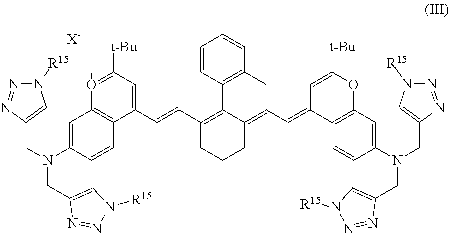
wherein each R¹⁵ comprises a hydrophilic group.

34. The compound of claim 33, wherein the hydrophilic group comprises a carboxylic acid group, an azide-functionalized peptide for targeting, a group that enhances cell permeability, a water solubilizing group or an ionic group.

35. (canceled)

36. The compound of claim 33, wherein each R¹⁵ is independently selected from:

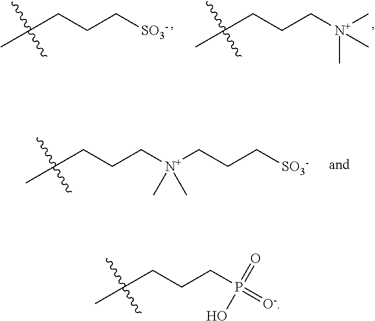

37. The compound of claim 33, wherein the hydrophilic group comprises a hydrophilic oligomer or polymer.

38. (canceled)

39. The compound of claim 1, having a structure of formula IV: 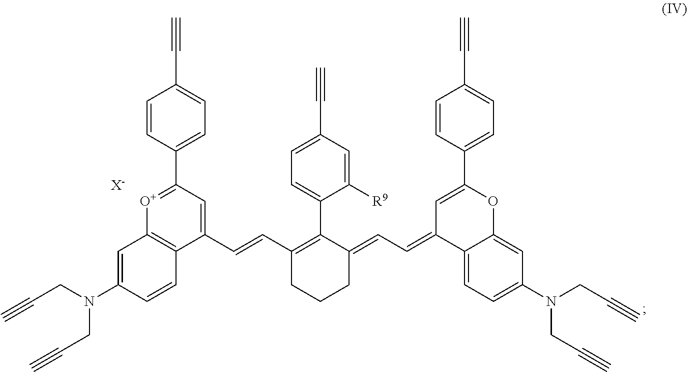
wherein X is selected from halide, BF₄⁻, and perchlorate, B(aryl)₄⁻, boron clusters, and TRISPHAT (tetrabutylammonium phosphorus(V) tris(tetrachlorocatecholate)); or a click conjugate thereof.

40. The compound of claim 1, having a structure selected from: 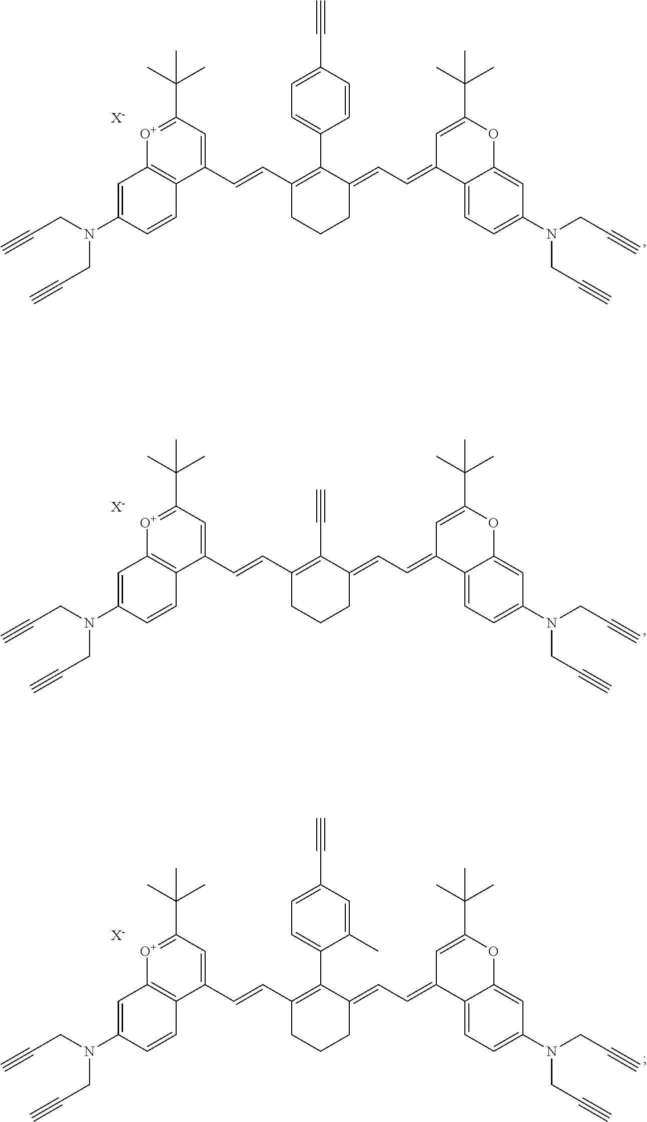
wherein X is selected from halide, BF₄⁻, perchlorate, BAr₄⁻, boron clusters, and TRISPHAT (tetrabutylammonium phosphorus(V) tris(tetrachlorocatecholate));
or a click conjugate thereof.

41. (canceled)

42. The compound of claim 1, selected from: 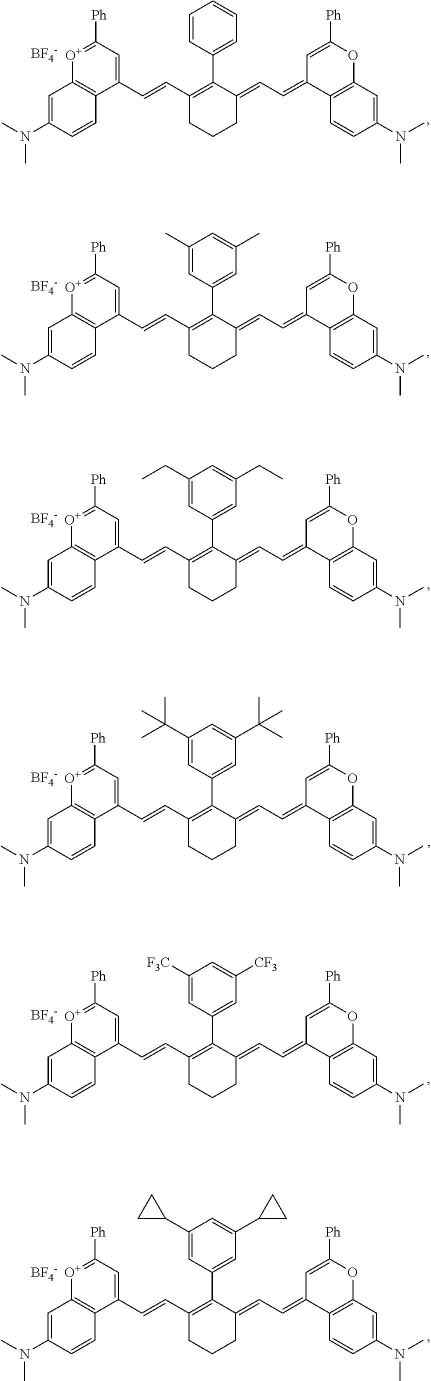 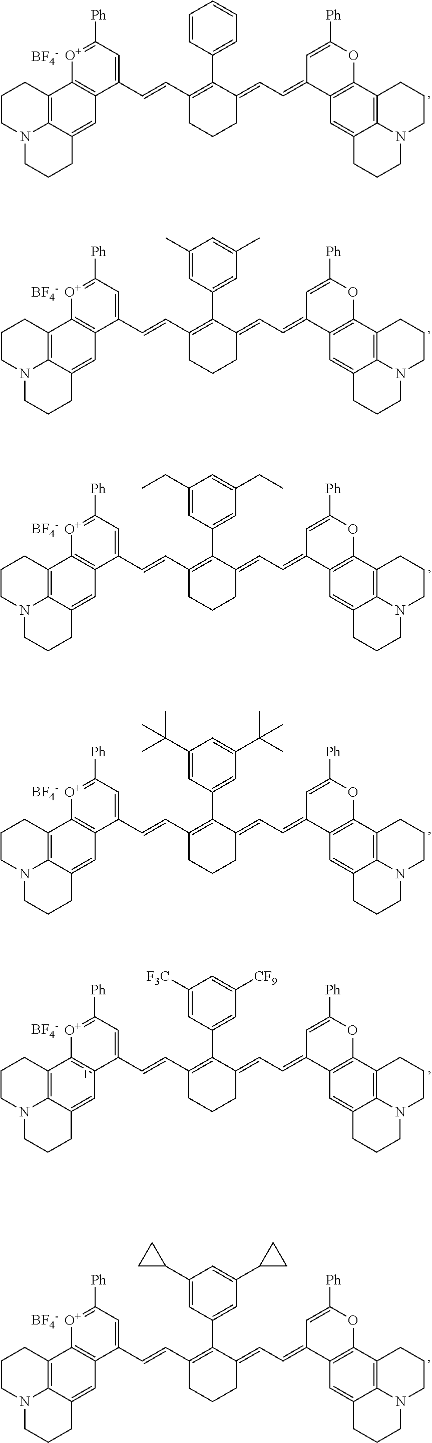 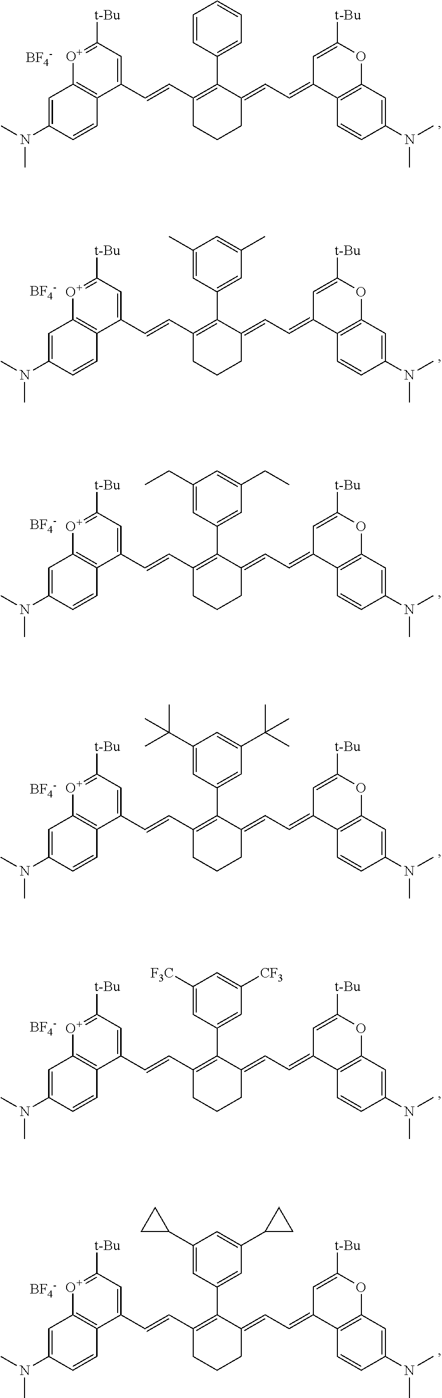 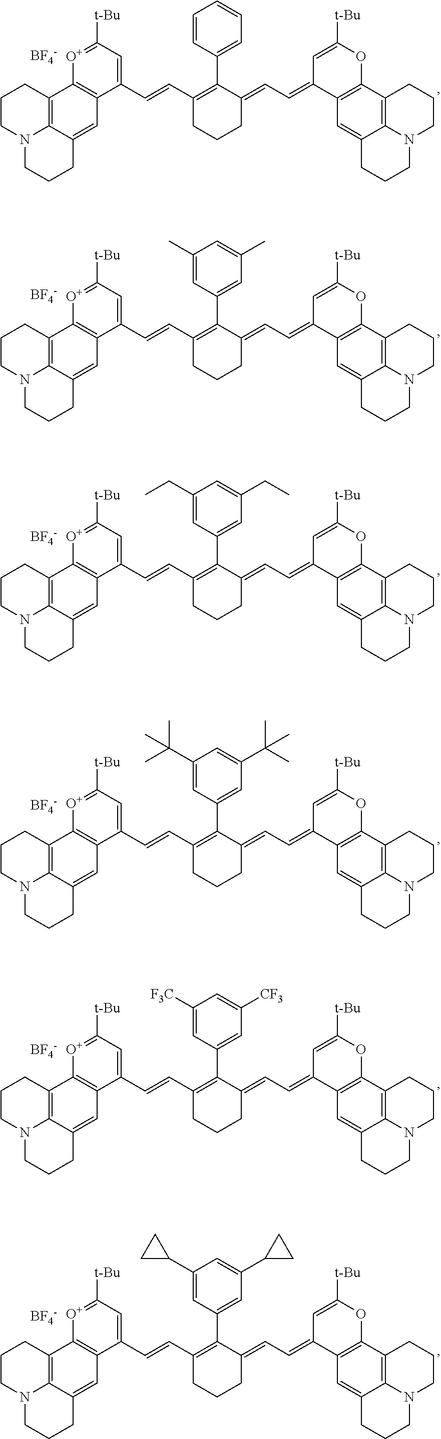 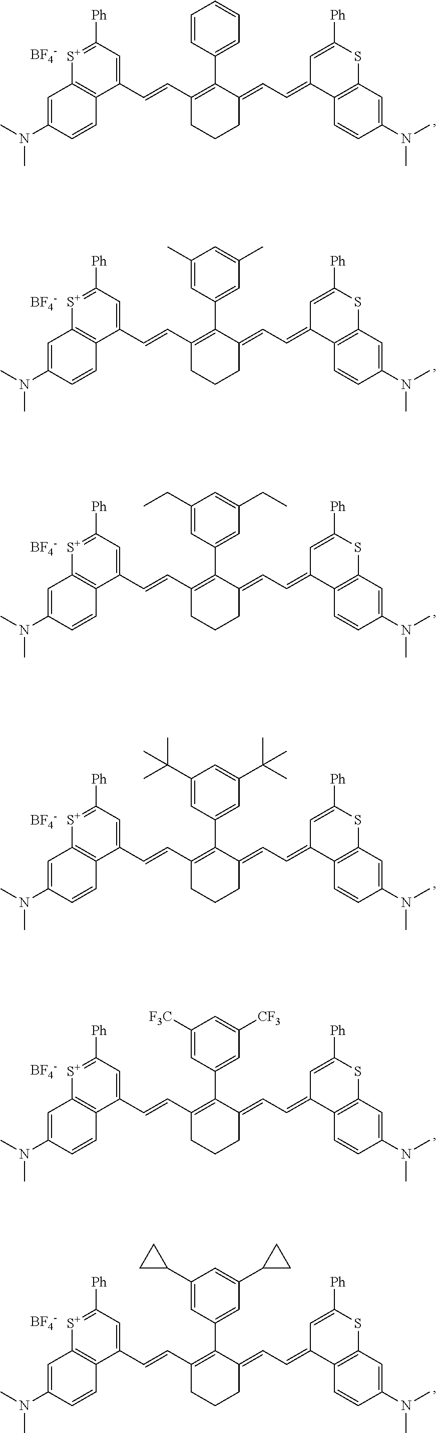 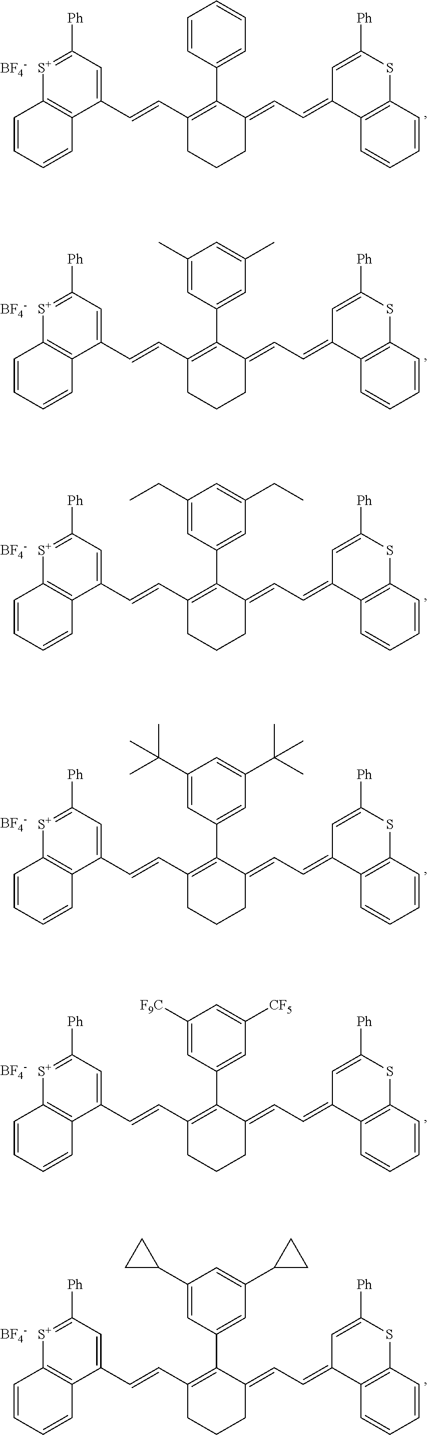 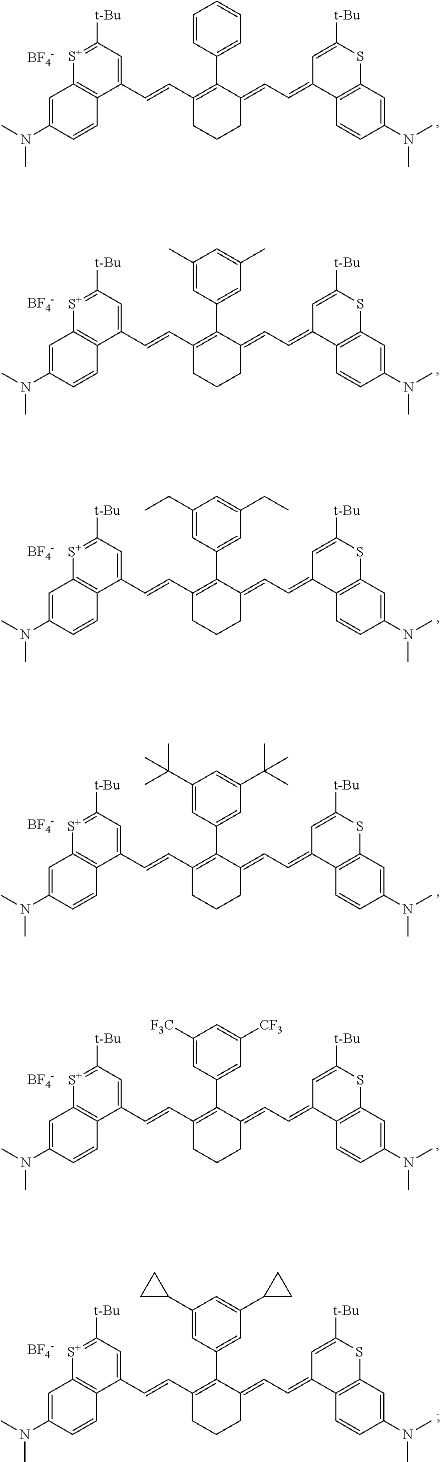
and salts wherein BF₄⁻ is replaced by an anion selected from halide, and perchlorate, BAr₄⁻, boron clusters, and TRISPHAT (tetrabutylammonium phosphorus(V) tris(tetrachlorocatecholate)).

43. A pharmaceutical composition comprising a compound of claim 1 and a pharmaceutically acceptable excipient.

44. A method of delivering a compound or composition of claim 1 to a living animal, comprising administering the compound or composition to the living animal.

45. A method of obtaining an image comprising:
illuminating a compound of claim 1 with excitation light, thereby causing the compound to emit fluorescence; and
detecting the fluorescence.

46-50. (canceled)
